SEQUENCE LISTING (29 sequences)

SEQ ID NO: 1 - DNA, 741 bp, synthetic construct
  source 1..741: mol_type = other DNA
gaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccttcttccacggcctacatgcaactgagctccctgacctcggaggactccgcagtgtactactgtgctcgcatgtggtactacggcacctactatttcgactattggggccaggggactactctgaccgtgtcctctgccgccgggggaggcggttctgggggtggtggatctggagggggtggctcccaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgacc

SEQ ID NO: 2 - protein, 247 aa, synthetic construct
EVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAAGGGGSGGGGSGGGGSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLT

SEQ ID NO: 3 - DNA, 741 bp, synthetic construct
  source 1..741: mol_type = other DNA
caggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccgggggaggcggttctgggggtggtggatctggagggggtggctccgaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccatcttccacggcctacatgcaactgagctccctgacctcggaggactccgcagtgtactactgtgctcgcatgtggtactacggcacctactatttcgactattggggccaggggactactctgaccgtgtcctctgccgcc

SEQ ID NO: 4 - protein, 247 aa, synthetic construct
QAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTGGGGSGGGGSGGGGSEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAA

SEQ ID NO: 5 - DNA, 1119 bp, synthetic construct
  source 1..1119: mol_type = other DNA
aaccccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatttgataatctagatagtagatagaatagtag

SEQ ID NO: 6 - protein, 363 aa, synthetic construct
NPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 7 - DNA, 339 bp, synthetic construct
  source 1..339: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 8 - protein, 113 aa, synthetic construct
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 9 - DNA, 300 bp, synthetic construct
  source 1..300: mol_type = other DNA
aacggatccggaggaggagggagcggaggaggagggagctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcatacgcatgataacctaggtagtagatagaatagtag

SEQ ID NO: 10 - protein, 90 aa, synthetic construct
NGSGGGGSGGGGSSPTEPSSKTGNPRHLHVLIGTSVVKIPFTILLFFLLHRWCSNKKNAAVMDQEPAGNRTVNSEDSDEQDHQEVSYAYA

SEQ ID NO: 11 - DNA, 324 bp, synthetic construct
  source 1..324: mol_type = other DNA
aacggatccggaggaggagggagcggaggaggagggagcgtcacaggaaacccttcaaatagttggccttcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcatgataacctaggtagtagatagaatagtag

SEQ ID NO: 12 - protein, 98 aa, synthetic construct
NGSGGGGSGGGGSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 13 - DNA, 804 bp, synthetic construct
  source 1..804: mol_type = other DNA
aaccggatccgtggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacccctttgggttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctcgagagagtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagatagtagatagaatagtag

SEQ ID NO: 14 - protein, 259 aa, synthetic construct
NRIRGVTVSSALSNSIMYFSHFVPVFLPAKPTTTPAPRPPTPAPTIASQPLSLRPEACRPAAGGAVHTRGLDPFGFWVLVVVGGVLACYSLLVTVAFIIFWVRSKRSRLLHSDYMNMTPRRPGPTRKHYQPYAPPRDFAAYRSLERVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 789 bp, synthetic construct
  source 1..789: mol_type = other DNA
aaccggatccgtggggtcaccgtctcctcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagatagtagatagaatagtag

SEQ ID NO: 16 - protein, 254 aa, synthetic construct
NRIRGVTVSSALSNSIMYFSHFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 1968 bp, synthetic construct
  source 1..1968: mol_type = other DNA
atggctcttcctgtgaccgcgttgctactgcccctggcgctgctcctgcacgccgctcgcccggaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccttcttccacggcctacatgcaactgagctccctgacctcggaggactccgcagtgtactactgtgctcgcatgtggtactacggcacctactatttcgactattggggccaggggactactctgaccgtgtcctctgccgccgggggaggcggttctgggggtggtggatctggagggggtggctcccaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccctgcagaaactgaatgaacagaaactgattagcgaagaagacctgaaccccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatttgataatctagatagtagatagaatagtag

SEQ ID NO: 18 - protein, 646 aa, synthetic construct
MALPVTALLLPLALLLHAARPEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAAGGGGSGGGGSGGGGSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTLQKLNEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 19 - DNA, 1968 bp, synthetic construct
  source 1..1968: mol_type = other DNA
atggctcttcctgtgaccgcgttgctactgcccctggcgctgctcctgcacgccgctcgcccgcaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccgggggaggcggttctgggggtggtggatctggagggggtggctccgaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccatcttccacggcctacatgcaactgagctccctgacctcggaggactccgcagtgtactactgtgctcgcatgtggtactacggcacctactatttcgactattggggccaggggactactctgaccgtgtcctctgccgccctgcagaaactgaatgaacagaaactgattagcgaagaagacctgaaccccgggggaggaggagggagcgggggaggaggcagcggcgggggaggctctggaggaggagggagcggatccatggatatccagatgacccagtccccgagctccctgtccgcctctgtgggcgatagggtcaccatcacctgccgtgccagtcaggacatccgtaattatctgaactggtatcaacagaaaccaggaaaagctccgaaactactgatttactatacctcccgcctggagtctggagtcccttctcgcttctctggttctggttctgggacggattacactctgaccatcagcagtctgcaaccggaagacttcgcaacttattactgtcagcaaggtaatactctgccgtggacgttcggacagggcaccaaggtggagatcaaaggcggcggcggaagtggaggaggaggctcaggcggaggagggagcgaggttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttagtacctacaaccagaagttcaaggaccgtttcactataagcgtagataaatccaaaaacacagcctacctgcaaatgaacagcctgcgtgctgaggacactgccgtctattattgtgctagaagcggatactacggcgatagtgactggtattttgacgtgtggggtcaaggaaccctggtcaccgtctcctcgactagtggcggaggaggatcactcgagagcggacaggtgctgctggaatccaatatcaaagtcctgcccacttggtctacccccgtgcagcctatggctctgattgtgctgggaggagtcgcaggactgctgctgtttatcgggctgggaattttcttttgcgtgcgctgccggcaccggagaaggcaggccgagcgcatgagccagatcaagcgactgctgagcgagaagaaaacctgtcagtgtccccatagattccagaagacctgttcacccatttgataatctagatagtagatagaatagtag

SEQ ID NO: 20 - protein, 646 aa, synthetic construct
MALPVTALLLPLALLLHAARPQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTGGGGSGGGGSGGGGSEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAALQKLNEQKLISEEDLNPGGGGGSGGGGSGGGGSGGGGSGSMDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 21 - DNA, 1566 bp, synthetic construct
  source 1..1566: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaggcagcggcgagggcagaggctccctgctgacctgcggggacgtggaagagaatcctggccctggcaccagccgtacgatggctcttcctgtgaccgcgttgctactgcccctggcgctgctcctgcacgccgctcgcccggaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccttcttccacggcctacatgcaactgagctccctgacctcggaggactccgcagtgtactactgtgctcgcatgtggtactacggcacctactatttcgactattggggccaggggactactctgaccgtgtcctctgccgccgggggaggcggttctgggggtggtggatctggagggggtggctcccaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccctgcagaaactgaatgaacagaaactgattagcgaagaagacctgaacggatccggaggaggagggagcggaggaggagggagctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcatacgcatgataacctaggtagtagatagaatagtag

SEQ ID NO: 22 - protein, 512 aa, synthetic construct
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYKGSGEGRGSLLTCGDVEENPGPGTSRTMALPVTALLLPLALLLHAARPEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAAGGGGSGGGGSGGGGSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTLQKLNEQKLISEEDLNGSGGGGSGGGGSSPTEPSSKTGNPRHLHVLIGTSVVKIPFTILLFFLLHRWCSNKKNAAVMDQEPAGNRTVNSEDSDEQDHQEVSYAYA

SEQ ID NO: 23 - DNA, 1590 bp, synthetic construct
  source 1..1590: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaaggcagcggcgagggcagaggctccctgctgacctgcggggacgtggaagagaatcctggccctggcaccagccgtacgatggctcttcctgtgaccgcgttgctactgcccctggcgctgctcctgcacgccgctcgcccggaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccttcttccacggcctacatgcaactgagctccctgacctcggaggactccgcagtgtactactgtgctcgcatgtggtactacggcacctactatttcgactattggggccaggggactactctgaccgtgtcctctgccgccgggggaggcggttctgggggtggtggatctggagggggtggctcccaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccctgcagaaactgaatgaacagaaactgattagcgaagaagacctgaacggatccggaggaggagggagcggaggaggagggagcgtcacaggaaacccttcaaatagttggccttcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcatgataacctaggtagtagatagaatagtag

SEQ ID NO: 24 - protein, 520 aa, synthetic construct
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYKGSGEGRGSLLTCGDVEENPGPGTSRTMALPVTALLLPLALLLHAARPEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAAGGGGSGGGGSGGGGSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTLQKLNEQKLISEEDLNGSGGGGSGGGGSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 25 - DNA, 1653 bp, synthetic construct
  source 1..1653: mol_type = other DNA
atggctcttcctgtgaccgcgttgctactgcccctggcgctgctcctgcacgccgctcgcccggaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccttcttccacggcctacatgcaactgagctccctgacctcggaggactccgcagtgtactactgtgctcgcatgtggtactacggcacctactatttcgactattggggccaggggactactctgaccgtgtcctctgccgccgggggaggcggttctgggggtggtggatctggagggggtggctcccaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccctgcagaaactgaatgaacagaaactgattagcgaagaagacctgaaccggatccgtggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacccctttgggttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccctcgagagagtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagatagtagatagaatagtag

SEQ ID NO: 26 - protein, 542 aa, synthetic construct
MALPVTALLLPLALLLHAARPEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAAGGGGSGGGGSGGGGSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTLQKLNEQKLISEEDLNRIRGVTVSSALSNSIMYFSHFVPVFLPAKPTTTPAPRPPTPAPTIASQPLSLRPEACRPAAGGAVHTRGLDPFGFWVLVVVGGVLACYSLLVTVAFIIFWVRSKRSRLLHSDYMNMTPRRPGPTRKHYQPYAPPRDFAAYRSLERVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1638 bp, synthetic construct
  source 1..1638: mol_type = other DNA
atggctcttcctgtgaccgcgttgctactgcccctggcgctgctcctgcacgccgctcgcccggaggtgcagctccagcagagcggcgcggagctggttaaacctggcgcttccgtgaagctgtcatgcaaagcttcgggctacacgttcaccagctattggatgcattgggtcaagcagaggcccggccgggggttggagtggatcggtcgcattgaccccaacggtggcggaaccaagtacaacgagaaatttaagtccaaggcaacacttacagtcgataagccttcttccacggcctacatgcaactgagctccctgacctcggaggactccgcagtgtactactgtgctcgcatgtggtactacggcacctactatttcgactattggggccaggggactactctgaccgtgtcctctgccgccgggggaggcggttctgggggtggtggatctggagggggtggctcccaggccgtggtgacccaggagtctgcgctgacgaccagcccaggggagaccgtcaccctgacttgccgaagttcgaccggcgccgtcacaacctctaattacgccaactgggtgcaggagaagcccgatcacctgttcaccggcctcattggaggcaccaacaaccgcgccccgggcgtaccagcgcgtttttcaggcagcctcatcggcaacaaggccgcactgaccatcaccggtgctcagactgaagacgaagccatctacttctgtgcgctttggtactccaatcacttggtgttcggcggcggcacgaagctcactgtgctgaccctgcagaaactgaatgaacagaaactgattagcgaagaagacctgaaccggatccgtggggtcaccgtctcctcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagatagtagatagaatagtag

SEQ ID NO: 28 - protein, 845 aa, synthetic construct
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIFTSGSGEGRGSLLTCGDVEENPGPGTSGTMALPVTALLLPLALLLHAARPEVQLQQSGAELVKPGASVKLSCKASGYTFTSYWMHWVKQRPGRGLEWIGRIDPNGGGTKYNEKFKSKATLTVDKPSSTAYMQLSSLTSEDSAVYYCARMWYYGTYYFDYWGQGTTLTVSSAAGGGGSGGGGSGGGGSQAVVTQESALTTSPGETVTLTCRSSTGAVTTSNYANWVQEKPDHLFTGLIGGTNNRAPGVPARFSGSLIGNKAALTITGAQTEDEAIYFCALWYSNHLVFGGGTKLTVLTLQKLNEQKLISEEDLNRIRGVTVSSALSNSIMYFSHFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 53 aa, synthetic construct
  MOD_RES 30: note = X is D-amino acids
  MOD_RES 34: note = X is D-amino acids
LLYSGLYGLYLSERGLYLASPLCHALPHEXSERXARGLTYRLLEULTRPLSER

(19) United States
(12) Patent Application Publication
(10) Pub. No.: US 2026/0248852 A1
(43) Pub. Date: Aug. 27, 2026

(54) SYNTHETIC ANTIGEN RECEPTORS FOR COVALENT IMMUNE RECRUITERS AND METHODS AND USES THEREOF

(71) Applicant: McMaster University, Hamilton (CA)

(72) Inventors: Nickolas Serniuck, Oakville (CA); Benjamin Lake, Ancaster (CA); Anthony Rullo, Hamilton (CA); Allyson Moore, Maple (CA); Jonathan Bramson, Oakville (CA); Eden Kapcan, Hamilton (CA)

(21) Appl. No.: 18/870,553

(22) PCT Filed: Jun. 2, 2023
(86) PCT No.: PCT/CA2023/050757

§ 371 Date: Nov. 29, 2024

Related U.S. Application Data
(60) Provisional application No. 63/348,744, filed on Jun. 3, 2022.

(51) Int. Cl. A61K 35/17; A61K 40/11; A61K 40/31; A61K 40/32; A61P 35/00; C07K 14/705; C07K 16/28; C07K 16/44; C12N 15/86

(52) U.S. Cl. CPC A61K 35/17; A61K 40/11; A61K 40/31; A61K 40/32; A61P 35/00; C07K 14/7051; C07K 14/70514; C07K 14/70521; C07K 14/70578; C07K 16/2809; C07K 16/44; C12N 15/86; A61K 2239/13; A61K 2239/17; A61K 2239/21; A61K 2239/22; C07K 2317/622; C12N 2710/10043; C12N 2740/10043; C12N 2740/15043; C12N 2750/14143

(57) ABSTRACT

The present description relates to synthetic antigen receptors for use with covalent immune recruiters (CIRs) comprising an donor moiety, a covalent binding group, and a target binding domain (TBD), the synthetic antigen receptor comprising an extracellular acceptor domain and an effector domain, wherein the extracellular acceptor domain is capable of binding to the donor moiety of the CIR. The present description also includes engineered cells comprising the synthetic antigen receptors, methods and uses thereof, for example, for recognition of target cells by immune cells comprising the synthetic antigen receptor functionalized with a CIR.

### CROSS-REFERENCE TO RELATED APPLICATIONS

This application claims the benefit of priority to U.S. Provisional Application No. 63/348,744, filed Jun. 3, 2022, the contents of which are incorporated herein by reference in their entirety.

### INCORPORATION OF SEQUENCE LISTING

A computer readable form of the Sequence Listing “3244-P68393PC00_SequenceListing.XML” (48,044 bytes) created on Jun. 2, 2023, is herein incorporated by reference.

### FIELD

The present disclosure relates to synthetic antigen receptors comprising an extracellular acceptor domain and an effector domain, as well as cells comprising said receptors, and methods of using such receptors and cells.

### BACKGROUND

T cells play an important role in controlling tumor growth and present a powerful tool for cancer treatment given their ability to circulate throughout the body and “seek out and destroy” tumor deposits. The process of infusing cancer patients with tumor-targeting T cells is known as adoptive T cell therapy. Clinical success in a variety of hematologic and solid malignancies has proven that adoptive T cell therapy is a viable strategy for treating human cancers¹⁻⁵. In fact, since 2017, five T cell “drugs” have received regulatory approval (Kymriah™, Yescarta™, Tecartus™, Breyanzi™, and Abecma™). These approvals were based on pivotal trials that revealed complete tumor clearance in a large fraction of patients suffering from relapsed and refractory leukemias, lymphomas and myelomas—such outcomes are unprecedented in patients with cancer at this stage. Importantly, many of these patients remain tumor-free years later after a single administration of the drug.

A key challenge in adoptive T cell therapy is the source of T cells, as naturally occurring tumor-specific T cells are rare. Large numbers of tumor-targeted T cells can be generated by engineering T cells to express synthetic antigen receptors⁶⁻¹⁰. The most widely used synthetic antigen receptor is the chimeric antigen receptor (CAR; reviewed in¹¹), which can be engineered into T cells isolated from peripheral blood, facilitating the rapid production of large numbers of tumor-directed T cells. T cells engineered with CARs (termed CAR-T cells) have proven to be a powerful method for treating pediatric and adult blood cancers¹²⁻¹⁵ In fact, the five approved cell therapies described in the previous paragraph are CAR-T cell products. To understand CAR design, it is necessary to appreciate T cell signaling. Ligation of the T cell receptor (TCR) leads to activation of the T cell, production of cytokines and release of cytotoxic molecules. The TCR defines the target specificity of the T cell but does not transmit intracellular signals. Rather, the TCR associates with the CD3 proteins, which mediate signal transduction via immunoreceptor tyrosine-based activation motifs (ITAMs)¹⁶. Current generation CARs are typically composed of: (i) one, or more, ligand(s) for the target cells, (ii) a cytoplasmic domain carrying one, or more, immunoreceptor tyrosine-based activation motif(s) (ITAM) and (iii) one, or more, additional cytoplasmic signaling domains that deliver costimulatory and/or survival signals to the T cell¹¹.

The enthusiasm for CAR-T cells is mitigated by severe, potentially lethal, toxicities, which must be carefully managed to ensure patient survival¹⁷. Given that the CAR technology has been developed to function independently of the native T cell receptor, it is possible that CAR-T cells lack appropriate regulatory pathways that are associated with the TCR. As a strategy to incorporate natural signaling mechanisms into a synthetic antigen receptor for T cells, several groups have designed synthetic receptors that were purpose-built to co-opt endogenous T cell activation pathways mediated via the TCR^{8-10,18}. In multiple independent publications, TCR-dependent synthetic receptors have outperformed TCR-independent synthetic receptors in pre-clinical tumor models supporting further investigation of these promising alternatives to CAR T cells^{8-10,18}.

An additional synthetic antigen receptor has been developed based on surface receptors that signal via DAP12²⁷. T cell activation via this DAP12-associated receptor is TCR-independent, operates separately from the TCR-based signaling and also displays improved anti-tumor activity in conventional T cells when compared to CARs.

It is not possible at present to ascertain which synthetic antigen receptor is best and, the choice of receptor will likely be influenced by numerous factors, including the target on the tumor, the tumor location and the type of T cell used. Thus, multiple synthetic antigen receptor platforms must be considered for clinical application as it is not possible to know a priori which platform will best suit the needs of individual disease sites or treatment regimens. It is challenging and costly to design and develop fusion proteins for each receptor platform, and to test the suitability of each for the target cell.

Conventional synthetic antigen receptors are rigid and target specificity is typically limited to one, or two, tumor target(s). Given the vast number of potential targets^{19,20}, many synthetic receptors and, by extension, multiple individual T cell products will be required to apply this approach broadly. The T cell manufacturing process is costly, even when centralized, and generating multiple separate batches of T cells to cover the full spectrum of tumor targets becomes a major cost driver. Universal synthetic antigen receptors have been designed that can be programmed with target specificity after the engineered T cells are manufactured²¹. A universal synthetic antigen receptor employs an acceptor moiety which binds a molecular adapter that links the T cell to the tumor. By creating a library of adapters, a T cell engineered with a single universal synthetic antigen receptor can be directed against multiple tumor targets. This strategy offers the potential for tailoring the T cells to the antigenic repertoire of the tumor. Pre-clinical studies have confirmed the utility of this strategy in the treatment of solid tumor (breast cancer, ovarian cancer) xenograft models using a variety of donor moieties including biotin, FITC, PNE and SpyTag²²⁻²⁵. Engineering T cells with universal synthetic antigen receptors offers the promise of producing a single engineered T cell product that can be programmed in a dynamic fashion post-manufacturing, which should keep costs low, as the adapters are far less expensive to produce than engineered T cells and remove barriers to accessibility.

U.S. patent application Ser. No. 17/249,332 describes molecular adapters, termed covalent immune recruiters (CIRs)²⁶, that chemically link a targeting domain to an antibody molecule such as an IgG, which targeted antibody can be used to direct immune cells to targeting domain receptors on cancer cells.

### SUMMARY

The inventors describe herein a collection of synthetic antigen receptors that are purpose-built for covalent immune recruiters (CIRs) and demonstrate their use in directing T cells to lyse tumor cells. The inventors unexpectedly demonstrate that covalent binding of the molecular adapters to the universal synthetic antigen receptor is critical for optimal activation of these receptors, even at saturating concentrations of adapter that exceed the K_{d} for binding the universal antigen receptor.

The inventors describe herein a series of synthetic antigen receptors designed to be combined with covalent molecular adapters (aka covalent immune recruiters) to allow dynamic targeting of engineered immune cells against the target defined by the adapter. The examples provided illustrate the use of this technology in a specific class of immune cell, the T cell, and for a specific application, cancer treatment. However, these examples should not be considered limiting and a person of ordinary skill in the art would be able to readily adapt these tools to other immune cells, including Natural Killer Cells, Natural Killer T cells, B cells, and macrophage, among others. Moreover, a person of ordinary skill in the art would able to adapt these tools for the treatment of other disease states, including autoimmunity, allergy and transplant rejection.

Described herein is the development of synthetic antigen receptors that can be covalently modified by Covalent Immune Recruiters (CIRs) to exert control over immune recognition when expressed in eukaryotic cells, and particularly in immune cells such as T cells. The resulting functionalized cells are able to affect immune recognition of model targets including tumor proteins on human cells, and induce a cytotoxic immune response targeting cells expressing the target tumor protein. Accordingly, functionalized cells comprising a synthetic antigen receptor, modified by a CIR, are useful tools for triggering a cytotoxic response to target cells.

The synthetic antigen receptors described herein comprise an extracellular acceptor domain for interacting with a donor moiety on the CIR, and an effector domain for effecting cellular signaling, in particular for activating an immune response, for example a cytotoxic response, a pro-inflammatory response, or an anti-inflammatory response in the cell expressing the receptor. The CIR comprises a donor moiety, a covalent binding group for covalently linking the CIR to the synthetic receptor, and a target binding domain for binding a ligand on the target cell, for example a tumor cell. Suitable CIRs for use with synthetic antigen receptors wherein the acceptor domain comprises an antibody fragment are described for example in U.S. patent application Ser. No. 17/249,332.

An aspect described herein includes a synthetic antigen receptor comprising: an extracellular acceptor domain; and an effector domain; for use with a covalent immune recruiter (CIR) comprising a donor moiety, a covalent binding group, and a target binding domain, wherein the extracellular acceptor domain of the synthetic antigen receptor is capable of binding to the donor moiety of the CIR.

In an embodiment, the extracellular acceptor domain comprises an antibody or antibody fragment, optionally an scFv.

In an embodiment, the antibody fragment is an anti-DNP scFv and the donor moiety is DNP, optionally the scFv comprises a variable heavy chain fragment (VH) and a variable light chain fragment (VL) in a VH-VL orientation, optionally the scFv comprises an amino acid sequence of SEQ ID NO: 2 or a functional variant thereof.

In an embodiment, the synthetic antigen receptor is a T cell antigen coupler (TAC)-based synthetic antigen receptor, a DNAX activation protein of 12 kDa (DAP12)-based synthetic antigen receptor, or a chimeric antigen receptor (CAR)-based synthetic antigen receptor.

In an embodiment, the synthetic antigen receptor is a TAC-based synthetic antigen receptor and the effector domain comprises an extracellular CD3ε binding domain, a transmembrane domain, and a CD4 cytoplasmic domain.

In an embodiment, the effector domain comprises an amino acid sequence of SEQ ID NO: 6 or a functional variant thereof.

In an embodiment, the synthetic antigen receptor comprises an amino acid sequence of SEQ ID NO: 18 or a functional variant thereof.

In an embodiment, the synthetic antigen receptor is a DAP12-based synthetic antigen receptor and the effector domain comprises a KIR2DS2 transmembrane and hinge domain, an NKp44 transmembrane and hinge domain, a TREM-1 transmembrane and hinge domain, or a TREM-2 transmembrane and hinge domain.

In an embodiment, the effector domain comprises an amino acid sequence of SEQ ID NO: 10 or 12, or a functional variant thereof.

In an embodiment, the synthetic antigen receptor further comprises a DAP12 signaling protein, optionally comprising an amino acid sequence of SEQ ID NO: 8 or a functional variant thereof.

In an embodiment, the synthetic antigen receptor comprises an amino acid sequence of SEQ ID NO: 22 or 24, or a functional variant thereof.

In an embodiment, the synthetic antigen receptor is a CAR-based synthetic antigen receptor and the effector domain comprises a transmembrane domain and an intracellular CD28 or CD137 signaling domain.

In an embodiment, the effector domain comprises an amino acid sequence of SEQ ID NO: 14 or 16, or a functional variant thereof.

In an embodiment, the synthetic antigen receptor comprises an amino acid sequence of SEQ ID NO: 26 or 28, or a functional variant thereof.

An aspect includes an engineered eukaryotic cell comprising a synthetic antigen receptor described herein.

In an embodiment, the cell is an immune cell.

In an embodiment, the immune cell is selected from lymphocytes, monocytes, polymorphonuclear cells, erythrocytes and megakaryocytes.

In an embodiment, the immune cell is selected from T cells, B cells, NK cells, macrophages, neutrophils, basophils, eosinophils and red blood cells.

In an embodiment, the T cell is selected from T cells with as T cell receptors (ABT cells), T cells with γδ T cell receptors (GDT cells), Mucosal-associated invariant T cells (MAIT cells) and natural killer T cells (NKT cells).

In an embodiment, the cell is further engineered to express one or more additional signaling components, optionally DAP12.

An aspect includes a method of generating a functionalized cell, the method comprising: providing an engineered cell comprising a synthetic antigen receptor described herein; and contacting the cell with a CIR comprising a donor moiety that binds the acceptor domain, a covalent binding group, and a target binding domain under conditions to allow binding of the donor moiety and acceptor domain and covalent attachment of the covalent binding group to the synthetic antigen receptor, thereby generating a functionalized cell.

A further aspect includes a functionalized cell generated by contacting an engineered cell comprising a synthetic antigen receptor described herein with a CIR comprising a donor moiety that binds the acceptor domain, a covalent binding group, and a target binding domain under conditions to allow binding of the donor moiety and acceptor domain and covalent attachment of the covalent binding group to the synthetic antigen receptor.

Another aspect includes a nucleic acid encoding a synthetic antigen receptor described herein.

In an embodiment, the extracellular acceptor domain comprises an scFv encoded by SEQ ID NO: 1 or a functional variant thereof.

In an embodiment, the effector domain is encoded by SEQ ID NO: 5, 7, 9, 11, 13, or 15, or a functional variant thereof.

In an embodiment, the nucleic acid comprises a sequence of SEQ ID NO: 17, 19, 21, 23, 25, or 27, or a functional variant thereof.

An aspect includes a method of treating a disease, disorder or condition that is treatable by immunotherapy, comprising administering to a subject in need thereof a therapeutically effective amount of a) an engineered cell comprising a synthetic antigen receptor described herein, and a CIR comprising a donor moiety that binds the acceptor domain, a covalent binding group, and a target binding domain; b) functionalized cell generated by contacting an engineered cell comprising a synthetic antigen receptor described herein with a CIR comprising a donor moiety that binds the acceptor domain, a covalent binding group, and a target binding domain under conditions to allow binding of the donor moiety and acceptor domain and covalent attachment of the covalent binding group to the synthetic antigen receptor; or c) a nucleic acid encoding a synthetic antigen receptor described herein and a CIR comprising a donor moiety that binds the acceptor domain, a covalent binding group, and a target binding domain.

In an embodiment, the disease, disorder, or condition that is treatable by immunotherapy is a cancer, an autoimmune disease, allergy, or transplant rejection.

In an embodiment, the cells are autologous.

In an embodiment, the cells are allogenic.

A further aspect includes a method of generating an engineered cell comprising a synthetic antigen receptor for use with a covalent immune recruiter (CIR) comprising a donor moiety, a covalent binding group, and a target binding domain, the method comprising introducing into the cell a nucleic acid encoding a synthetic antigen receptor described herein.

In an embodiment, the nucleic acid is introduced into the cell by viral transduction.

In an embodiment, the virus is selected from adenovirus, adeno associated virus, retrovirus and lentivirus.

In an embodiment, the nucleic acid is introduced into the cell by transfection.

In an embodiment, the nucleic acid is plasmid DNA or synthetic RNA.

Another aspect includes a kit comprising: a) an engineered cell comprising a synthetic antigen receptor described herein, and optionally a CIR; b) a functionalized cell described herein; or c) a nucleic acid encoding a synthetic antigen receptor described herein, and optionally a CIR.

Other features and advantages of the present description will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF DRAWINGS

The embodiments of the present disclosure will now be described in greater detail with reference to the attached drawings in which:

FIG. 1 shows the design of the universal synthetic antigen receptor. The universal synthetic antigen receptor is introduced into T cells via gene transfer and expressed on the cell surface. Synthetic molecular adapters, comprising donors that bind the universal receptor and tumor ligands, couple the universal synthetic antigen receptor to the target cell. The acceptor domain on the receptor is capable of binding multiple molecular adapters via a common donor moiety enabling a single engineered T cell product to be directed against multiple tumor antigens.

FIG. 2A-2B shows the design and mechanism of action of a Covalent Immune Recruiter (CIR). (FIG. 2A) The covalent immune recruiter or CIR is a molecular adapter with a covalent binding group that serves to promote covalent linkage of the tumor binding domain to an immune receptor. Conventional molecular adapters that lack the covalent binding group (alternatively termed the antibody labeling domain) are herein termed non-covalent immune recruiters or (N)CIR. (FIG. 2B) Example of covalent binding resulting from antibody binding to CIR. Nucleophilic attack via a proximal lysine in the binding site. Labeling via acylimidazole chemistry results in ejection of the donor moiety from the CIR.

FIGS. 3A-3C show an illustration of the TCR and the design and expression of the TAC receptor that binds DNP described in this disclosure. (FIG. 3A) The T cell receptor (TCR) comprises a multimeric structure with 8 subunits (2 CD3ε subunits, CD3α subunit, CD3β subunit, CD3γ subunit, CD3δ subunit, and 2 CD3ζ subunits). (FIG. 3B). The traditional tri-functional T cell antigen coupler (TAC) receptor comprises a tumor binding domain, an extracellular domain that binds to CD3ε, and the transmembrane and cytoplasmic domains of human CD4. FIG. 3B shows a schematic of DNP-specific TAC receptor with an scFv against DNP in the position of the tumor binding domain. (FIG. 3C) Transduction evaluation via flow cytometry of the anti-DNP TAC engineered T Cells.

FIG. 4A-4D shows selective binding of a DNP-CIR to T cells engineered with the DNP-TAC. (FIG. 4A) Structure of a Desthiobiotin-containing CIR (DNP-Acyl Imidazole Desthiobiotin) and the non-covalent version (DNP-Desthiobiotin) (FIG. 4B) Demonstration that DNP-Acyl Imidazole Desthiobiotin (CIR) can covalently bind and label anti-DNP scFv containing T-cells whereas DNP-Desthiobiotin [(N)CIR]cannot. (FIG. 4C) Demonstration of rapid covalent binding of DNP-Acyl Imidazole Desthiobiotin (CIR) across a range of concentration and complete absence of binding by DNP-Desthiobiotin [(N)CIR]. (FIG. 4D) reveals that while all Anti-DNP TACT cells are loaded with DNP-Acyl Imidazole-Desthiobiotin at 1 uM within 30 minutes as shown in FIG. 4C, the total load of DNP-Acyl Imidazole-Desthiobiotin continues to increase over several hours demonstrating that maximal loading of the DNP-TAC T cells takes several hours.

FIG. 5A-5D demonstrates the functional advantage of molecular adapters that can covalently bind to the Anti-DNP TAC T cells shown in FIG. 3. (FIG. 5A) Structure of DNP-Acyl Imidazole-Glutamate Urea Lysine (PSMA-CIR) and DNP-Glutamate-Urea-Lysine [PSMA-(N)CIR]. (FIG. 5B) Measurement of Anti-DNP TAC T cell activation based CD69 upregulation in response to the combination of PSMA-CIR and PSMA-expressing tumor cells. Anti-DNP TAC T cells were incubated with PSMA-CIR, PSMA-(N)CIR or media alone and stimulated with either wild type HEK-293 cells or PSMA transduced HEK-293 cells followed by measurement of the activation marker, CD69. Only the PSMA-CIR was capable of stimulating Anti-DNP TAC T cell activation in the presence of PSMA. (FIG. 5C) Assessment of cytokine production in response to the combination of PSMA-CIR and PSMA-expressing tumor cells. Anti-DNP TAC T cells were incubated with PSMA-CIR, PSMA-(N)CIR or media alone and stimulated with either wild type HEK-293 cells or PSMA transduced HEK-293 cells followed by measurement of the cytokines, interferon-gamma (IFN-g) and tumor necrosis factor-alpha (TNF-a). (FIG. 5D) Assessment of killing of PSMA-expressing prostate cancer cells. Anti-DNP TAC T cells were co-cultured with PSMA-positive LNCaP cells in the presence of varying concentrations of PSMA-CIR and PSMA-(N)CIR. The PSMA-CIR promoted killing of the LNCaP cells at low nM concentrations whereas the PSMA-(N)CIR did not promote killing of LNCaP cells at any concentration.

FIG. 6A-6B shows the effect of heavy and light chain orientation on the capacity of the Anti-DNP TAC to bind CIRs. The anti-DNP SPE7 scFv was cloned into TAC receptors in two orientations: Variable Heavy-Variable Light (VHVL) and Variable Light-Variable Heavy (VLVH). (FIG. 6A) T cells engineered with anti-DNP TAC receptors were loaded with DNP-Acyl Imidazole Desthiobiotin (CIR) at various concentrations and the amount of CIR was assessed by flow cytometry. The VHVL form of the SPE7 scFv bound the greatest amount of CIR. (FIG. 6B) Anti-DNP TAC T cells were co-cultured with PSMA-positive LNCaP cells in the presence of DNP-Acyl Imidazole-Glutamate Urea Lysine (PSMA-CIR). The TAC T cell carrying the VHVL form of the SPE7 scFv displayed the greatest level of tumor control.

FIGS. 7A-7C show an illustration of the structures of the DAP12-based synthetic antigen receptor (DAP12-SAR) and chimeric antigen receptor described in this disclosure along with confirmation of expression on primary T cells by flow cytometry. (FIG. 7A) The DAP12-based synthetic antigen receptor is comprised of the anti-DNP scFv in VH-VL orientation linked to the transmembrane and extracellular domain of KIR2DS2. (FIG. 7B) The chimeric antigen receptor (CAR) is a single polypeptide that comprises the anti-DNP scFv in VH-VL configuration, an intracellular costimulatory signaling domain derived from CD28 or CD137, and the cytoplasmic portion of CD3ζ. (FIG. 7C). Transduction of human GDT cells with both receptors reveals high levels of receptor expression.

FIG. 8A-8B shows selective binding of a DNP-CIR to T cells engineered with the Anti-DNP DAP12-SAR and the Anti-DNP CD28 CAR. (FIG. 8A) Demonstration of rapid covalent binding of DNP-Acyl Imidazole Desthiobiotin (CIR) across a range of concentration and minimal binding of DNP-Desthiobiotin [(N)CIR] to T cells engineered with both synthetic antigen receptors. (FIG. 8B) reveals that T cells engineered with the Anti-DNP DAP12-SAR bind to greater amounts of CIR than T cells engineered with the Anti-DNP CD28 CAR.

FIG. 9A-9D demonstrates that both the Anti-DNP DAP12 SAR-GDT-cells and Anti-DNP CD28 CAR-GDT-cells can be functionally redirected using the PSMA-CIR specific to kill PSMA-expressing LNCaP cells. (FIG. 9A) Anti-DNP DAP12 SAR-GDT-cells and Anti-DNP CD28 CAR GDT-cells were co-cultured with PSMA-positive LNCaP cells in the presence of 125 nM PSMA-CIR or PSMA-(N)CIR. While the Anti-DNP DAP12 SAR-GDT-cells only suppressed tumor growth in the presence of the PSMA-CIR, the Anti-DNP CD28 CAR GDT-cells displayed tumor growth control in the presence of either PSMA-CIR or PSMA-(N)CIR. (FIG. 9B) The Anti-DNP DAP12 SAR-GDT-cells displayed comparable levels of tumor killing over range of PSMA-CIR concentrations as low as 15 nM. No killing by Anti-DNP DAP12 SAR-GDT-cells was observed with (N)CIR concentrations of 125 nM or lower and only weak killing was observed at the highest concentration of PSMA-(N)CIR (250 nM). The Anti-DNP CD28 CAR GDT-cells also displayed comparable levels of tumor killing over range of PSMA-CIR concentrations as low as 15 nM. In contrast, tumor killing by Anti-DNP CD28 CAR GDT-cells diminished at lower levels of PSMA-(N)CIR. (FIG. 9C) shows the tumor growth curves corresponding to the 125 nM and 15.63 nM data shown in FIG. 10B. (FIG. 9D) In an independent experiment, Anti-DNP DAP12 SAR GDT-cells and Anti-DNP CD28 CAR GDT-cells were co-cultured with PSMA-positive LNCaP cells in the presence of 10 nM or 1 nM of PSMA-CIR or PSMA-(N)CIR. In this circumstance, the CIR/(N)CIR concentrations were limiting revealing a striking dichotomy between the ability of the PSMA-CIR to effectuate killing of LNCaP cells by the Anti-DNP CD28 CAR GDT-cells and the inability of the PSMA-(N)CIR to effectuate killing of LNCaP cells by the Anti-DNP CD28 CAR GDT-cells at limiting concentration. Collectively, the data demonstrating an advantage of covalent binding of the molecular adapters when present at limiting concentrations.

FIG. 10 shows the absolute requirement of covalent binding when the T cells were preincubated with the PSMA-CIR/(N)CIR prior to co-culture with LNCaP cells. Anti-DNP DAP12 SAR GDT-cells and Anti-DNP CD28 CAR GDT-cells were incubated in the presence of 1 uM PSMA-CIR or 1 uM PSMA-(N)CIR for 6 hours, washed and co-cultured with LNCaP cells. Only the T cells incubated with the PSMA-CIR retained the ability to kill LNCaP cells.

FIG. 11A-11D shows the utility of an alternate chemistry, SuFEx, to covalent binding to DNP-specific T-cells. (FIG. 11A) Structures of the DNP-Sufex-Bitoin, DNP-Sufex-uPAR peptide CIR, and DNP-uPAR peptide (N)CIR. (FIG. 11B) Demonstration that DNP-SuFEx-Desthiobiotin can covalently bind and label DNP-TAC T cells whereas DNP-Desthiobiotin [(N)CIR]cannot. Non-engineered T cells and 200×DNP competitor were included as controls to display the covalency and specificity of the CIR. DNP-Acyl Imidazole-Desthiobiotin CIR was also included as a positive control. (FIG. 11C) Demonstration that Anti-DNP DAP12 SAR GDT-cells can be redirected using the DNP-SuFEx-based uPAR-CIR to kill uPAR-positive A172 cells, whereas the corresponding uPAR-(N)CIR could not. (FIG. 11D) Demonstration that Anti-DNP CD28 CAR GDT-cells can be redirected using the DNP-SuFEx-based uPAR-CIR to kill uPAR-positive A172 cells, whereas the corresponding uPAR-(N)CIR showed minimal cytotoxicity.

FIG. 12A-12B shows the utility of SuFEx chemistry to functionally redirect DNP-specific ABT cells toward uPAR expressing glioblastoma cells. (FIG. 12A) Assessment of cytokine production via intracellular staining in response to the combination of uPAR-expressing tumor cells (A172) and either uPAR-CIR with SuFEx chemistry or uPAR-(N)CIR. T cells were engineered with one of the synthetic antigen receptors (SARs): Anti-DNP TAC, Anti-DNP DAP12 SAR or Anti-DNP CD28 CAR. There SAR-engineered T cells were subsequently co-cultured with either wild type A172 or A172 cells where uPAR was removed by CRISPR/Cas9 (A172KOA) cells. To promote T cell:tumor cell interaction, either the uPAR-CIR or uPAR-(N)CIR was included at 100 nM. As a negative control, T cells and tumor cells were co-cultured in media alone. Following stimulation, the ABT cells were stained intracellularly for both interferon gamma (IFNg) and TNF alpha (TNFa). Activation of all three engineered T cells was found to be CIR dependent as only CIR reprogrammed cells showed an increase in cytokine production over the media controls. CD4 and CD8 T-cell populations showed the same results; the total percent of CD8-positive T cells producing cytokines was used for this figure. (FIG. 12B) Assessment of killing of uPAR-expressing glioblastoma cancer cells. SAR-engineered T cells were co-cultured with uPAR-positive A172 cells in the presence of varying concentrations of uPAR-CIR and uPAR-(N)CIR. These data reveal that Anti-DNP TAC, Anti-DNP DAP12 SAR, and Anti-DNP CD28 CAR ABT-cells can be redirected using the DNP-SuFEx-based uPAR-CIR to kill uPAR-positive A172 cells, whereas the corresponding uPAR-(N)CIR could not direct killing of any engineered T cell.

FIG. 13A-13B shows the increased effectiveness of CIR redirection on early T cell activation and signal strength with various engineered receptors in ABT cells. (FIG. 13A) Target-dependent activation of T cells was measured through CD69 and Nur77. ABT cells were engineered with either Anti-DNP TAC (TAC), Anti-DNP DAP12 SAR (DAP12-SAR), and Anti-DNP CD28 CAR (CD28-CAR). T cells were subsequently co-cultured with 293 cells that express PSMA (293-PSMA), 293 cells that do not express PSMA (293-WT) or left in media only. 100 nM PSMA-CIR was added to certain co-cultures. T cell activation, measured by upregulation of CD69 and Nur77, only occurred in the presence of CIR and PSMA-expressing target cells confirming that these activation parameters occur in a CIR-dependent and target-dependent manner. The same results were observed for both CD4 and CD8 positive T cells; CD4-positive cells were used as the representative population in this figure. (FIG. 13B) Anti-DNP TAC, Anti-DNP DAP12 SAR, and Anti-DNP CD28 CAR engineered ABT cells were incubated with 10 nM and 1 nM of either PSMA-CIR or PSMA-(N)CIR in the presence of 293-PSMA engineered cells. Levels of surface CD69 and intracellular Nur77 were analyzed through flow cytometry to determine differences in activation and downstream signal strength. It was observed that CIR facilitates increased activation and downstream signal strength when compared to (N)CIR in all receptors at both 10 nM and 1 nM with receptor biology influencing differences. The same results were seen in both CD4 and CD8 positive T cells; CD8-positive cells were used as the representative population in this figure.

FIG. 14A-14C demonstrates that receptor biology can influence T cell function when covalent molecular adapters are used in conjunction engineered ABT cells. The data in this figure were generated with ABT cells engineered with either Anti-DNP TAC, Anti-DNP DAP12 SAR, and Anti-DNP CD28 CAR. (FIG. 14A) Cytokine production elicited by CIR and (N)CIR is receptor-dependent. Cytokine production was measured as a response to the combination of PSMA-CIR (CIR) or PSMA-(N)CIR [(N)CIR] and PSMA-expressing tumor cells. Engineered T cells were co-cultured with 293 cells that express PSMA (293-PSMA), 293 cells that do not express PSMA (293-WT) or left in media only. Various concentrations of PSMA-CIR or PSMA-(N)CIR were added to the co-cultures (1, 10, 100 nM). At the end of the co-culture, the percentage of cells producing cytokines interferon-gamma and tumor necrosis factor-alpha was assessed by flow cytometry. Increased cytokine production was observed in the presence CIR compared to (N)CIR for all engineered T cells. The receptor influenced the importance of covalency where T cells engineered with TAC receptor were only activated by the CIR whereas T cells engineered with Anti-DNP DAP12 SAR or Anti-DNP CD28 CAR could be activated by both CIR and (N)CIR, but the strongest activation was mediated by the CIR. The same results were seen for both CD4 and CD8 positive T cells, CD8-positive cells were used as the representative population in this figure. (FIG. 14B) Cytotoxicity elicited by CIR and (N)CIR is receptor-dependent. Engineered T cells were co-cultured with PSMA-positive LNCaP cells in the presence of varying concentrations of PSMA-CIR and PSMA-(N)CIR. T cells engineered with Anti-DNP TAC or Anti-DNP DAP12 SAR were critically dependent upon covalent attachment and little cytotoxicity was observed in the co-culture with (N)CIR. T cells engineered with Anti-DNP CD28 CAR displayed comparable cytotoxicity at high concentrations of CIR/(N)CIR but covalency improved cytotoxicity at low concentrations. (FIG. 14C) Proliferation elicited by CIR and (N)CIR is receptor-dependent. Engineered T cells were labeled with cell trace violet (CTV) and co-cultured with 293 cells that express PSMA (293-PSMA), 293 cells that do not express PSMA (293-WT) or left in media only. Various concentrations of PSMA-CIR or PSMA-(N)CIR were added to the co-cultures (1, 10, 100 nM). The percentage of cells which entered division was used as a readout of T cell proliferation. T cells engineered with Anti-DNP TAC or Anti-DNP DAP12 SAR were critically dependent upon covalent attachment and little proliferation was observed in the co-culture with (N)CIR. T cells engineered with Anti-DNP CD28 CAR displayed comparable cytotoxicity at high concentrations of CIR/(N)CIR but covalency improved cytotoxicity at low concentrations. The same results were seen in both CD4 and CD8 positive T cells, CD8-positive cells were used as the representative population in this figure. Overall, the work shows that the covalency of the CIR allows for a functional advantage when reprogramming all the tested engineered receptors in ABT cells.

### DETAILED DESCRIPTION

### III. I. Definitions

Unless otherwise indicated, the definitions and embodiments described in this and other sections are intended to be applicable to all embodiments and aspects of the present disclosure herein described for which they are suitable as would be understood by a person skilled in the art. Each aspect so defined may be combined with any other aspect or aspects unless clearly indicated to the contrary. In particular, any feature described herein may be combined with any other feature or features described herein.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

As used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this description and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

The term “suitable” as used herein means that the selection of the particular compound or conditions would depend on the specific manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms.

The term “aryl” as used herein, whether it is used alone or as part of another group, refers to carbocyclic groups containing at least one aromatic ring and contains either 6 to 20 carbon atoms.

The term “amine” or “amino,” as used herein, whether it is used alone or as part of another group, refers to groups of the general formula NR′R″, wherein R′ and R″ are each independently selected from hydrogen or C₁₋₆alkyl.

The term “amino acid” as used herein refers to an organic compound comprising amine (—NH₂) and carboxylic acid (—COOH) functional groups, along with a side-chain specific to each amino acid. The common elements of an amino acid are carbon, hydrogen, oxygen and nitrogen, though other elements are found in the side-chains of certain amino acids, including S and Se. Unless otherwise specified, an amino acid referenced herein is one of the 23 proteinogenic amino acids, that is amino acids that are precursors to proteins, and are incorporated into proteins during translation.

The term “nucleic acid” or “nucleic acid molecule” and its derivatives, as used herein, are intended to include unmodified DNA or RNA or modified DNA or RNA. For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “expression cassette” refers to a DNA molecule encoding an RNA or protein operably linked to a promoter and a polyadenylation signal, such that certain portions of the expression cassette are capable of being transcribed into RNA (such as antisense RNA, Long non-coding RNA or for the genome of a virus such as a lentivirus) and/or as a messenger RNA that is subsequently translated into protein by cellular machinery.

The term “operably linked” as used herein refers to a relationship between two components that allows them to function in an intended manner. For example, where a reporter gene is operably linked to a promoter, the promoter actuates expression of the reporter gene.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters for the expression cassette will be known to the skilled person. In some embodiments, the promoter is an inducible promoter. Examples of inducible promoters include, without limitation, a tetracycline response element (TRE) (e.g. Tet-ON or Tet-OFF systems), ponA-inducible expression systems (Agilent Technologies), or cumate-inducible promoters such as CuO (System Biosciences). In some embodiments, the promoter is a constitutive promoter. Examples of constitutive promoters include human Ubiquitin C (UBC), human Elongation Factor 1alpha (EF1A), human phosphoglycerate kinase 1 (PGK), simian virus 40 early promoter (SV40) (GeneBank accession number J02400.1), cytomegalovirus immediate-early promoter (CMV), chicken b-Actin promoter coupled with CMV early enhancer (CAG) and EF1-HTLV.

The term “transcription termination site” as used herein refers generally to a polyadenylation signal (pA) that terminates transcription of messenger RNA (mRNA). Suitable pAs may be derived from any organism and are known to the skilled person. Examples of pA signals include, without limitation, rabbit beta-globin pA (GeneBank accession number K03256), SV40 late polyA, hGH polyA and strong bovine growth hormone pA (BGHpA) (GeneBank accession number M57764.1).

The term “antibody” as used herein is intended to include chimeric and humanized antibodies and binding fragments thereof, including for example a single chain Fab fragment, Fab′2 fragment, or single chain Fv fragment. Humanized or other chimeric antibody fragments may include sequences from one or more than one isotype, class, or species. Further, these antibodies are typically produced as single domain antibody fragments, or as single chain antibodies in which the heavy and light chains are linked by a spacer. The antibodies may include sequences from any suitable species including human.

The term “antibody fragment” or “binding fragment” as used herein is intended to include without limitations Fab, Fab′, F(ab′)2, scFab, scFv, dsFv, ds-scFv, Fc-fusion proteins, dimers, minibodies, diabodies, and multimers thereof, multispecific antibody fragments and Domain Antibodies. Fab, Fab′ and F(ab′)2, scFv, dsFv, ds-scFv, Fc-fusion proteins, dimers, minibodies, diabodies, bispecific antibody fragments and other fragments can also be synthesized by recombinant techniques.

The term “functional variant” as used herein includes modifications of the polypeptide or nucleic acid sequences disclosed herein that perform substantially the same function as the polypeptide molecules disclosed herein in substantially the same way. For example, the functional variant may comprise sequences having at least 80%, or at least 90%, or at least 95% sequence identity to the sequences disclosed herein. The functional variant may also comprise conservatively substituted amino acid sequences of the sequences disclosed herein.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences are maximally aligned by IMGT or other (e.g. Kabat or Chothia numbering conventions). The terms “IMGT numbering” or “ImMunoGeneTics database numbering”, which are recognized in the art, refer to a system of numbering amino acid residues which are more variable (i.e. hypervariable) than other amino acid residues in the heavy and light chain variable regions of an antibody, or antigen binding portion thereof. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Accordingly, IMGT and other alignment systems can also be used to identify or annotate CDRs in an antibody sequence.

In another embodiment, the functional variant nucleic acid sequences comprise de-generate codon substitutions or codon-optimized nucleic acid sequences. The term “degenerate codon substitution” as used herein refers to variant nucleic acid sequences in which the second and/or third base of a codon is substituted with a different base that does not result in a change in the amino acid sequence encoded therein. The term “codon-optimized” as used herein refers to a variant nucleic acid molecule comprising one or more degenerate codon substitutions that reflect the codon usage bias of a particular organism.

The following symbol:

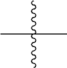

is used in chemical structures herein to represent a point of covalent attachment of a group to another group.

The term “aq.” As used herein refers to aqueous.

DMSO as used herein refers to dimethylsulfoxide.

NHS as used herein refers to N-hydroxysuccinimide ester.

PSMA as used herein refers to prostate specific membrane antigen.

NK as used herein refers to natural killer cells.

TBD as used herein refers to target cell binding domain.

CIR as used herein refers to covalent immune recruiter.

(N)CIR or NCIR as used herein refers to non-covalent immune recruiter.

DNP as used herein refers to dinitrophenyl.

GDT cells as used herein refers to gamma delta T cells or T cells with γδ receptors, and includes, without limitation, γ9δ2 T cells.

ABT as used herein refers to alpha beta T cells or T cells with αβ receptors.

MAIT as used herein refers to Mucosal-associated invariant T cells.

NKT as used herein refers to natural killer T cells.

The term “linker” or “linker group” as used herein refers to any molecular structure that joins two or more other molecular structures together and that is compatible with a biological environment.

The term “compatible with a biological environment” as used herein it is meant that the chemical group or molecule is stable in, and/or does not denature or perturb, other molecules present in biological systems.

The term “biological systems” as used herein means any of a wide variety of systems which comprise proteins, enzymes, organic compounds, inorganic compounds, other sensitive biopolymers including DNA and RNA, and includes complex systems such as whole or fragments of plant, animal and microbial cells.

The term “subject” as used herein includes all members of the animal kingdom including mammals, and suitably refers to humans. Thus the methods and uses of the present disclosure are applicable to both human therapy and veterinary applications. Optionally, the term “subject” includes mammals that have been diagnosed with cancer or are in remission. In one embodiment, the term “subject” refers to a human having, or suspected of having, cancer.

The term “subject in need thereof” refers to a subject that could benefit from the method(s) or treatment(s) described herein, and optionally refers to a subject with cancer, or optionally a subject with increased risk of cancer, such as a subject previously having cancer, a subject with a precancerous syndrome or a subject with a strong genetic disposition.

The term “increased risk of cancer” as used herein means a subject that has a higher risk of developing a particular cancer than the average risk of the population. A subject may have a higher risk due to previously having had said particular cancer and or having a genetic risk factor for said particular cancer or exhibit a pre-cancer syndrome.

The term “administered” or “administering” as used herein means administration of a therapeutically effective amount of one or more synthetic antigen receptors, nucleic acids, engineered or functionalized cells, and/or CIRs for use with the synthetic antigen receptors described herein, or a composition described herein to a cell either in cell culture or in a patient.

The term “immune response” as used herein can refer to activation of either or both the adaptive and innate immune system cells such that they shift from a dormant resting state to a state in which they are able to elaborate molecules typical of an active immune response.

The phrase “inducing an immune response” as used herein refers to a method whereby an immune response is activated. The phrase “enhancing an immune response” refers to augmenting an existing immune response.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt of a compound, wherein molecules of a suitable solvent are incorporated in the crystal lattice

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with one or more synthetic antigen receptors, nucleic acids, engineered or functionalized cells, and/or CIRs for use with the synthetic antigen receptors described herein to prevent recurrence. Treatment methods comprise administering to a subject a therapeutically effective amount of one or more one or more synthetic antigen receptors, nucleic acids, engineered or functionalized cells, and/or CIRs for use with the synthetic antigen receptors described herein and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The term “disease, disorder or condition” as used herein refers to a disease, disorder or condition treatable by immunotherapy, such as by one or more synthetic antigen receptors, nucleic acids, engineered or functionalized cells, and/or CIRs for use with the synthetic antigen receptors described herein.

The term “immunotherapy” as used herein refers to the treatment of disease, disorder or condition by activating the immune system to produce or provoke an immune response.

The term “hapten” as used herein refers to a small molecule that can elicit an immune response only when attached to a large carrier such as a protein. The carrier may be one that also does not elicit an immune response by itself.

As used herein, the term “effective amount” or “therapeutically effective amount” means an amount of one or more synthetic antigen receptors, nucleic acids, engineered or functionalized cells, and/or CIRs for use with the synthetic antigen receptors described herein that is effective, at dosages and for periods of time necessary to achieve the desired result. For example, in the context of treating a disease, disorder or condition mediated or treatable by immunotherapy, an effective amount is an amount that, for example, provoke an immune response compared to without administration of the one or more synthetic antigen receptors, nucleic acids, engineered or functionalized cells, and/or CIRs for use with the synthetic antigen receptors described herein.

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells, as in a hematological cancer such as leukemia.

The term “cancer cell” refers a cell characterized by uncontrolled, abnormal growth and the ability to invade another tissue or a cell derived from such a cell. Cancer cells include, for example, a primary cancer cell obtained from a patient with cancer or cell line derived from such a cell. In one embodiment, the cancer cell is a hematological cancer cell such as a leukemic cell or a lymphoma cell.

The phrase “cancer burden” refers to the quantum of cancer cells or cancer volume in a subject. Reducing cancer burden accordingly refers to reducing the number of cancer cells or the cancer volume in a subject.

### II. Synthetic Antigen Receptors, Cells, and Compositions

Described herein is the development of synthetic antigen receptors useful for engineering eukaryotic cells, such as immune cells, to be functionalized with a target binding domain using molecular adapters, termed covalent immune recruiters (CIRs).

### Synthetic Antigen Receptors

The synthetic antigen receptors described herein comprise an extracellular acceptor domain (also referred to herein as an acceptor moiety), which can be bound by a cognate CIR or (N)CIR, and an effector domain which effects signaling in an engineered cell. In an embodiment, as shown in the Examples herein, a synthetic antigen receptor comprising an acceptor domain, such as an anti-DNP scFv, can be used to functionalize T cells using CIRs comprising a donor moiety, such as DNP.

The acceptor domain may be any suitable domain, including, but not limited to, antibodies and antibody fragments. As will be understood by the skilled person, the selection of an acceptor domain will depend on the donor moiety of the selected CIR or (N)CIR.

The effector domain may be based on any suitable signaling platform. For example, the effector domain may comprise components of a T cell antigen coupler (TAC), a DNAX activation protein of 12 kDa (DAP12)-based signaling platform, or a chimeric antigen receptor (CAR).

By way of example, a typical TAC (also known as a tri-functional TAC) comprises a tumor binding domain, an extracellular domain that binds to CD3ε, and the transmembrane and cytoplasmic domains of human CD4. In a TAC-based synthetic antigen receptor, the acceptor domain replaces the tumor binding domain of the TAC, and the effector domain comprises the remaining components of the TAC (e.g. an extracellular domain that binds to CD3ε, and the transmembrane and cytoplasmic domains of human CD4). In an embodiment, the effector domain comprises the sequence of SEQ ID NO: 6, or a functional variant thereof.

Similarly, a typical DAP12-based signaling platform comprises a target binding domain and a transmembrane and hinge domain that can interact with DAP12 and activate DAP12 signaling in the cell. The cell may be engineered to express DAP12, or may endogenously express DAP12. Suitable transmembrane and hinge domains include those from DAP12-associated receptors such as Killer Cell Immunoglobulin Like Receptor, Two Ig Domains And Short Cytoplasmic Tail 2 (KIR2DS2), Natural Killer Cell Activating Receptor p44 (NKp44; also known as Natural Cytotoxicity Triggering Receptor 2 or NCR2), triggering receptor expressed on myeloid cells (TREM)-1, or TREM-2. A DAP12-based synthetic antigen receptor comprises an acceptor domain in place of the target binding domain, and the effector domain comprises the remaining components of the platform (e.g. a KIR2DS2 transmembrane and hinge domain). In an embodiment, the effector domain comprises the sequence of SEQ ID NO: 10 or a functional variant thereof, or SEQ ID NO: 12 or a functional variant thereof. Cellular activation using DAP12-based synthetic antigen receptors is mediated via DAP12. Accordingly, in an embodiment, the cell endogenously expresses DAP12. In another embodiment, the cell is further engineered to express DAP12. In another embodiment, the synthetic antigen receptor further comprises DAP12. In this embodiment, the DAP12 is cleaved from the synthetic antigen receptor when in the cell. In an embodiment the synthetic antigen receptor further comprising DAP12 comprises the sequence of SEQ ID NO: 22 or 24 or a functional variant thereof. In an embodiment, DAP12 comprises the sequence of SEQ ID NO: 8.

As used herein, the term “CAR” refers to a chimeric antigen receptor. As known in the art, a CAR molecule typically comprises an extracellular antigen binding domain, a hinge region, a transmembrane domain, and one or more intracellular domains such as a co-stimulatory signaling domain, such as a CD28 or CD137 (also known as 4-1BB) signaling domain, and/or a CD3 zeta domain. In the context of the present description, a CAR-based synthetic antigen receptor comprises an acceptor domain in place of the antigen binding domain, and the effector domain comprises a hinge region, a transmembrane domain, and one or more intracellular domains (e.g. co-stimulatory signaling domain and/or CD3 zeta domain) of a typical CAR. In an embodiment, the effector domain comprises the sequence of SEQ ID NO: 14 or a functional variant thereof, or SEQ ID NO: 16 or a functional variant thereof.

Also provided herein are nucleic acids, optionally nucleic acid constructs, expression cassettes, or vectors, encoding the synthetic antigen receptors described herein. In various embodiments, the nucleic acid comprises one or more sequences selected from SEQ ID NOs: 1, 3, 5, 7, 9, 11, 13, 15, 17, 19, 21, 23, 25, and 27, and functional variants thereof.

It will be understood that a nucleic acid molecule or construct may be integrated into the genetic material of a cell, or may be incorporated into a vector or plasmid. Accordingly, in an aspect, the nucleic acids described herein may be provided in the form of one or more plasmids or vectors comprising a nucleic acid, including RNA, or expression cassette encoding the synthetic antigen receptors described herein, and/or in the form of a cell comprising one or more nucleic acids or expression cassettes encoding the synthetic antigen receptors described herein.

### Covalent Immune Recruiters

As used herein, the term “covalent immune recruiter” or “CIR” refers to a chemically synthetic compound of Formula I or a pharmaceutically acceptable salt and/or solvate thereof:

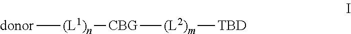

wherein
“donor” (also referred to as “donor moiety”) is a binding domain that interacts with the acceptor domain of the synthetic antigen receptor;
CBG is a covalent binding group comprising a functional group that forms a covalent bond with an amino acid in the synthetic antigen receptor that is proximal to the acceptor domain and the formation of the covalent bond results in elimination of the donor;
L¹ and L² are, independently, linker groups;
n and m are, independently, 0 or 1; and
TBD is a target binding domain.

### Donors

The donor moiety is a chemically synthetic molecule comprising a binding domain which binds to the acceptor domain of the synthetic antigen receptor. Any suitable donor moiety may be used depending on the specific acceptor domain of the synthetic antigen receptor. In an embodiment, the donor moiety is an antibody binding domain for example as described in U.S. patent application Ser. No. 17/249,332, and the acceptor domain comprises an antibody or antibody fragment which binds the donor moiety.

In an embodiment, the acceptor domain is an anti-dinitrophenyl (DNP) scFv. In an embodiment, the donor moiety comprises DNP for binding the anti-DNP scFv.

In an embodiment, the donor moiety comprises an electron deficient aryl or a carbohydrate. In an embodiment, the electron deficient aryl group is di- or trinitro phenyl. In an embodiment the carbohydrate comprises digalactose.

In an embodiment, the donor moiety comprises any of the hapten groups described in U.S. Pat. No. 9,296,708. Accordingly, the donor moiety group may be one of the following groups:
(1) a di- or trinitrophenyl group having the following structure:

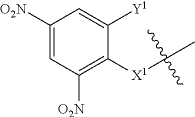

wherein Y¹ is H or NO₂;
X¹ is NR¹, O, CH₂, S(O), SO₂, SO₂O, OSO₂ or OSO₂O; and
R¹ is H, C₁₋₄alkyl or C(O)C₁₋₄alkyl;
(2) a bicyclic nitro-substituted aromatic group having the following structure:

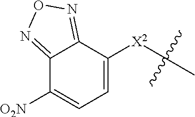

wherein X² is a bond, O, CH₂, NR² or S; and
R² is H, C₁₋₄alkyl or C(O)C₁₋₄alkyl;
(3) a galactose-containing carbohydrate having the following structure:

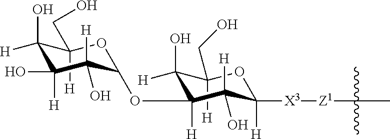

wherein X³ is CH₂, O, NR³ or S;
R³ is H or C₁₋₄alkyl; and
Z¹ is a bond, monosaccharide, disaccharide, oligosaccharide, glycoprotein or glycolipid; or
(4) a group having the following structure:

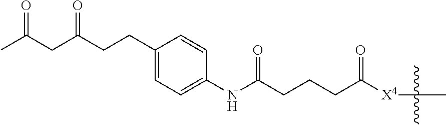

wherein X⁴ is O, CH₂ or NR⁴; and
R⁴ is H, C₁₋₄alkyl or C(O)C₁₋₄alkyl.

In an embodiment, X¹ is NR¹ and R¹ is H or C₁₋₃alkyl. In an embodiment Y¹ is H.

In an embodiment, X² is a bond or NR² and R² is H or C₁₋₃alkyl.

In an embodiment, X³ is O or NR³ and R³ is H or C₁₋₃alkyl. In an embodiment, Z¹ is a bond. In an embodiment, Z¹ is a monosaccharide or a disaccharide. In an embodiment, the monosaccharide is an aldose such as aldotriose (D-glyceraldehdye, among others), aldotetrose (D-erythrose and D-Threose, among others), aldopentose, (D-ribose, D-arabinose, D-xylose, D-lyxose, among others) or aldohexose (D-allose, D-altrose, D-Glucose, D-Mannose, D-gulose, D-idose, D-galactose and D-Talose, among others). In an embodiment, the monosaccharide is a ketose such as ketotriose (dihydroxyacetone, among others), ketotetrose (D-erythrulose, among others), ketopentose (D-ribulose and D-xylulose, among others) or ketohexose (D-Psicone, D-Fructose, D-Sorbose, D-Tagatose, among others). In an embodiment the monosaccharide is an aminosugar such as galactoseamine, sialic acid, N-acetylglucosamine, among others or a sulfosugar such as sulfoquinovose, among others. In an embodiment Z is a disaccharide such as sucrose (which may have the glucose optionally N-acetylated), lactose (which may have the galactose and/or the glucose optionally N-acetylated), maltose (which may have one or both of the glucose residues optionally N-acetylated), trehalose (which may have one or both of the glucose residues optionally N-acetylated), cellobiose (which may have one or both of the glucose residues optionally N-acetylated), kojibiose (which may have one or both of the glucose residues optionally N-acetylated), nigerose (which may have one or both of the glucose residues optionally N-acetylated), isomaltose (which may have one or both of the glucose residues optionally N-acetylated), β,β-trehalose (which may have one or both of the glucose residues optionally N-acetylated), sophorose (which may have one or both of the glucose residues optionally N-acetylated), laminaribiose (which may have one or both of the glucose residues optionally N-acetylated), gentiobiose (which may have one or both of the glucose residues optionally N-acetylated), turanose (which may have the glucose residue optionally N-acetylated), maltulose (which may have the glucose residue optionally N-acetylated), palatinose (which may have the glucose residue optionally N-acetylated), gentiobiluose (which may have the glucose residue optionally N-acetylated), mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose or xylobiose, among others. In an embodiment Z¹ is an oligosaccharide such as any sugar of three or more (up to about 100) individual sugar (saccharide) units as described above (i.e., any one or more saccharide units described above, in any order, especially including glucose and/or galactose units as set forth above), or for example, fructo-oligosaccharides, galactooligosaccharides or mannan-oligosaccharides ranging from three to about ten-fifteen sugar units in size. In an embodiment, Z¹ is a glycoprotein such as N-glycosylated or O-glycosylated glycoproteins, including the mucins, collagens, transferring, ceruloplasmin, major histocompatability complex proteins (MHC), enzymes, lectins, selectins, calnexin, calreticulin, or integrin glycoprotein lib/lia, among others. In an embodiment Z¹ is a glycolipid such as a glyceroglycolipid (galactolipids or sulfolipids) or a glycosphingolipid, such as cerebrosides, galactocerebrosides, glucocerebrosides (including glucobicaranateoets), gangliosides, globosides, sulfatides, glycophosphphingolipids or glycocalyx, among others.

In an embodiment, Z¹ is a bond or a glucose or glucosamine (such as N-acetylglucosamine). In an embodiment, Z¹ is linked to a galactose residue through a hydroxyl group or an amine group on the galactose of Gal-Gal, suitably a hydroxyl group.

In an embodiment, X⁴ is NR⁴ and R⁴ is H or C₁₋₃alkyl.

In an embodiment, the donor moiety is:

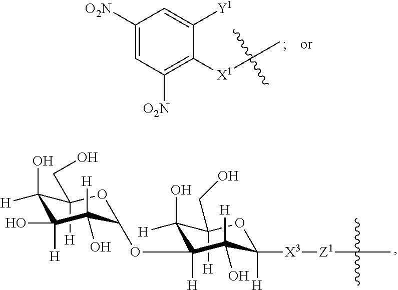

wherein
Y¹ is H or NO₂, suitably H; X¹ is NH or O, suitably NH;
X³ is O; and
Z¹ is a bond, a monosaccaride or a disaccharide, suitable a bond.

In an embodiment, the donor moiety is selected from a variety of monosaccharide or multivalent derivatives thereof recognized by several different serum carbohydrate specific antibodies such as anti-rhamnose (e.g. anti-rhamnose IgG 23F) and N-acetylglucosamine. In some embodiments, the donor moiety comprises synthetic ligands such as cyclic peptides that bind all serum IgG.

### Target Cell Binding Domains (TBD)

Target cell binding domains, or target binding domains (TBDs), comprise targeting moieties which are taken up and retained in a particular site of a subject such as a biological structure for example an organ or tissue or a pathological structure for example a tumor, with little or no accumulation and/or retention in non-target sites over a particular time period. In an embodiment, the TBD binds to a protein, for example a protein that is overexpressed in a disease, disorder or condition such as cancer. Targeting moieties are known and the selection of a suitable targeting moiety for a particular therapeutic use can be made by a person skilled in the art. Targeting moieties include, but are not limited to, small molecules such as protein binding compounds, enzyme inhibitors, receptor ligands, or pharmaceutical-like compounds.

In an embodiment, the TBD comprises a moiety that binds to antigens on the surface of a target cell. In an embodiment, the TBD is a glutamate urea ligand that binds to prostate specific membrane antigen (PSMA).

In an embodiment, the TBD comprises any of the target cell binding domain groups described in U.S. Pat. No. 9,296,708. Accordingly, the TBD group may be one of the following groups:

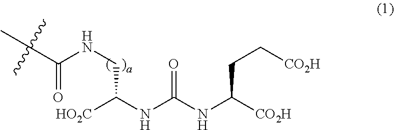

wherein a is an integer from 0 to 10, 1 to 15, 1 to 10, 1 to 8, or 1, 2, 3, 4, 5 or 6;

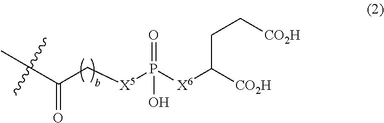

wherein X⁵ and X⁶ are independently CH₂, O, NH or S; and
b is an integer from 0 to 10, 1 to 15, 1 to 10, 1 to 8, or 1, 2, 3, 4, 5 or 6;

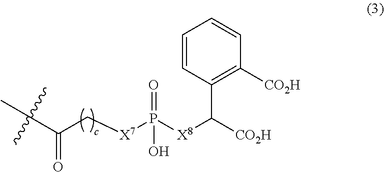

wherein X⁷ and X⁸ are independently CH₂, O, NH or S; and
c is an integer from 0 to 10, 1 to 15, 1 to 10, 1 to 8, or 1, 2, 3, 4, 5 or 6; or

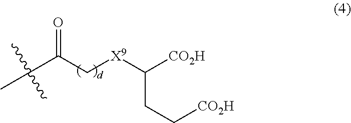

wherein X⁹ is O, CH₂, NR⁵, S(O), SO₂, SO₂O, OSO₂ or OSO₂O;
R⁵ is H, C₁₋₄alkyl or C(O)C₁₋₄alkyl; and
d is an integer from 0 to 10, 1 to 15, 1 to 10, 1 to 8, or 1, 2, 3, 4, 5 or 6.

In an embodiment, a, b, c, and d are independently 1, 2, 3, 4, 5 or 6, suitably 2, 3 or 4, more suitably 4.

In an embodiment, the TBD group is one of the following groups:

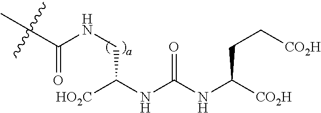

wherein a is 1, 2, 3, 4, 5 or 6; or

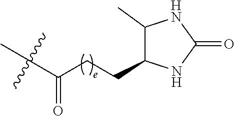

wherein e is 1, 2, 3, 4, 5 or 6.

In some embodiments, the TBD group comprises other tumor antigen binding ligands such as, without limitation, synthetic peptides against uPAR (e.g. L-Lys-Gly-Gly-L-Ser-Gly-L-Asp-L-Cha-L-Phe-D-Ser-D-Arg-L-Tyr-L-Leu-L-Trp-L-Ser as used herein) or HER2, or folate receptor binding molecules such as folate or methotrexate, or Toll-like receptor (TLR) agonists or PD-1/PD-L1 antagonists, The TBD may also include Integrin binding ligands such as the Knottin peptide and other RDG mimetics. The TBD may also include therapeutic antibodies, scFv, aptamers, and other biologics.

In some embodiments, the TBD group may be an antigen or epitope that binds to allergen-reactive B cells such as, without limitation, Ara h1, Ara h2, Fel d1 or other defined allergens, antigens and epitopes for allergen-reactive B cells.

In some embodiments, the TBD group may be an antigen or epitope that binds auto reactive B cells such, without limitation, citrullinated peptides, desmogleins or other antigens and epitopes for autoreactive B cells.

In some embodiments, the TBD may be a ligand for a donor MHC molecule that would be used to elicit a regulatory T cell response to suppress graft rejection.

### Covalent Binding Groups

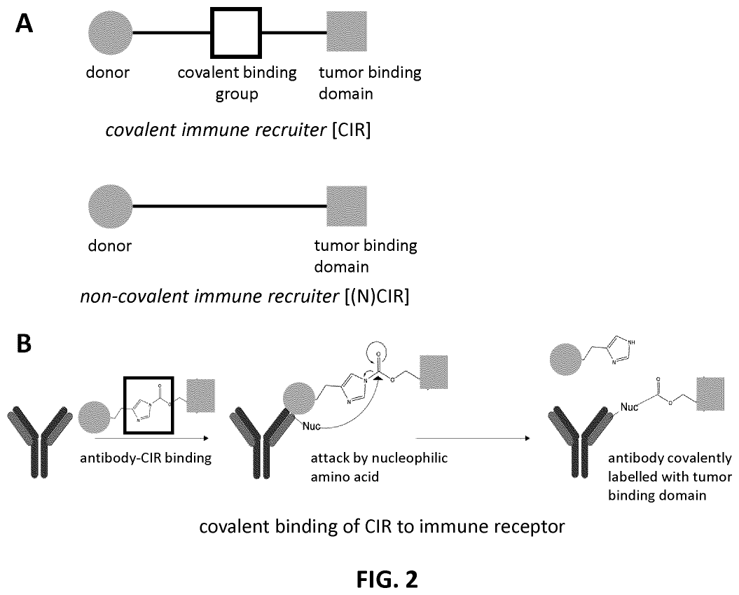

The covalent binding group (CBG) comprises a functional group that reacts with and forms a covalent bond with an amino acid in the synthetic antigen receptor, for example in the acceptor (e.g. antibody or antibody fragment), that is proximal to the donor moiety binding site. Depending on the CBG and type of reaction, the formation of the covalent bond may result in elimination of the donor moiety (See FIG. 2B).

The covalent binding group comprises a moiety that reacts with an amino acid in the acceptor domain (e.g. antibody or antibody fragment) to form a covalent bond with the acceptor domain. In an embodiment, the reaction results in elimination (or displacement) of the donor moiety. In an embodiment, the reaction does not result in elimination (or displacement) of the donor moiety. The amino acid in the acceptor domain is proximal to the binding site for the donor moiety. By “proximal” it is meant that the amino acid is located in an area that, when the CIR is bound to the acceptor domain via the donor moiety, the amino acid is in a spatial location to react with the covalent binding group. For example, the distance between the amino acid and the covalent binding group may be about 2 Å to about 10 Å.

In some embodiments, the covalent binding group comprises an electrophilic functional group that reacts with an amino acid nucleophile in a nucleophilic substitution reaction. In an embodiment, the amino acid nucleophile is an amine (NH₂) or a thiol (SH). In an embodiment, the electrophilic functional group in the covalent binding group comprises an imidazole group having the following structure:

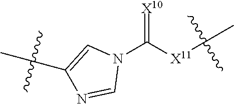

wherein
X¹⁰ is S, O or NR⁶;
X¹¹ is O or NR⁷; and
R⁶ and R⁷ are independently H or C₁₋₄alkyl.

In an embodiment, X¹⁰ and X¹¹ are both O.

In some embodiments, the covalent binding group comprises a sulfur(VI) fluoride exchange (SuFEx) group, for example sulfonyl fluoride.

The covalent binding group may also incorporate click chemistry handles to enable for a subsequent 2-step ligation of the target binding domain to the acceptor domain on the synthetic antigen receptor using “click” chemistry processes. Click chemistry is used in the art to describe a class of “bio-orthogonal” reactions which are often used for attaching a probe or substrate of interest to a specific biomolecule in a process called bioconjugation which can take place in vitro or directly in vivo. This class of biocompatible small molecule reactions may include, for example, [3+2] cycloadditions such as the Huisgen 1,3-dipolar cycloaddition, thiol-ene reactions, Diels-Alder reactions and inverse electron demand Diels-Alder reactions, [4+1] cycloadditions between isonitriles (isocyanides) and tetrazines. Use of suitable click chemistry would be well within the purview of a person skilled in the art. In some embodiments, the click chemistry is a copper catalyzed reaction of an azide and an alkyne to form a triazole.

A person skilled in the art would appreciate that there are many other functional groups that may be used in the covalent binding group. Such group would be compatible with a biological environment and would react with a functional group on an amino acid in an antibody to form a covalent bond, optionally wherein formation of the covalent bond results in elimination of the donor moiety.

### Linker Groups

A person of skill in the art would appreciate that the linkers L¹ and L² should have a length and spatial orientation appropriate to link the donor moiety with the covalent binding group and the covalent binding group with the TBD moiety. In some embodiments, the linker rigidity and length is tuned to maximize labeling kinetics and further comprises rigidifying elements such as carbocycles, heterocycles, aromatics and/or heteroaromatics.

Linkers may be any molecular structure that joins two or more other molecular structures together and that is compatible with a biological environment. In an embodiment, the linker moiety comprises at least one ester, amide, ether, thioether, thioamide, thioester and/or amine.

In an embodiment, L¹ and L², are independently, C₁₋₂₀ alkylene, optionally interrupted by triazolyl and/or one or more heteromoieties such as O, S, S(O), SO₂, OSO₂, SO₂O, OSO₂O, NR⁸, C(O), NHC(O), or C(O)NH, wherein R⁸ is H, C₁₋₄alkyl, or absent. In an embodiment, L¹ and L² are independently, a group having the following structure:

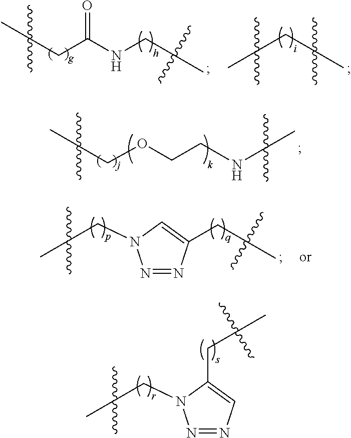

wherein, g, h, i, j, k, p, q, rand s are, independently, 1, 2, 3, 4, 5, 6, 7, 8, 9 or 10.

In an embodiment, j is 2 and k is 3. In an embodiment, g is 1 and h is 2. In an embodiment, i is 1, 2, 3, 4, 5, 6, 7, 8, 9 or 10. In an embodiment, p, q, rand s are, independently, 1, 2, 3 or 4.

In an embodiment, the linker groups are present, therefore n and m in the compounds of Formula I are both 1 and the compounds have the following structure:

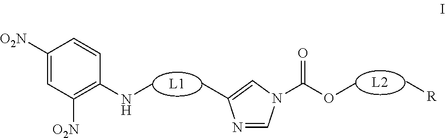

wherein
R is TBD.

In an embodiment, R is

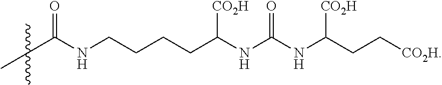 [SMILES: *C(=O)NCCCCC(NC(=O)NC(CCC(=O)O)C(=O)O)C(=O)O]

In an embodiment, the CIR is:

Structure

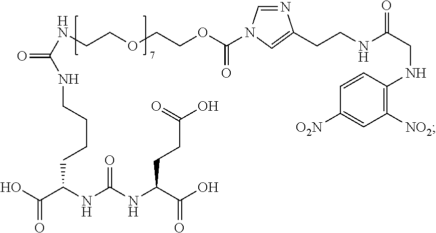

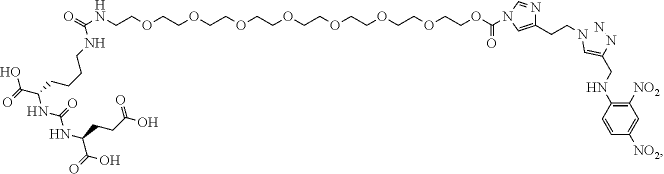 [SMILES: O=C(O)CC[C@H](NC(=O)N[C@@H](CCCCNC(=O)NCCOCCOCCOCCOCCOCCOCCOCCOC(=O)n1cnc(CCn2cc(CNc3ccc([N+](=O)[O-])cc3[N+](=O)[O-])nn2)c1)C(=O)O)C(=O)O]

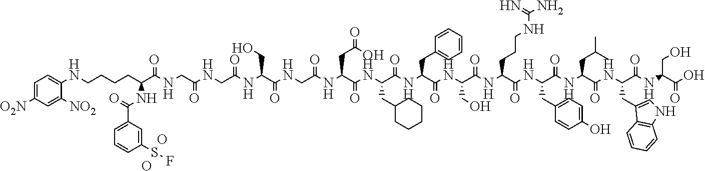 [SMILES: CC(C)C[C@H](NC(=O)[C@H](Cc1ccc(O)cc1)NC(=O)[C@H](CCCNC(=N)N)NC(=O)[C@H](CO)NC(=O)[C@H](Cc1ccccc1)NC(=O)[C@H](CC1CCCCC1)NC(=O)[C@H](CC(=O)O)NC(=O)CNC(=O)[C@H](CO)NC(=O)CNC(=O)CNC(=O)[C@H](CCCCNc1ccc([N+](=O)[O-])cc1[N+](=O)[O-])NC(=O)c1cccc(S(=O)(=O)F)c1)C(=O)N[C@@H](Cc1c[nH]c2ccccc12)C(=O)N[C@@H](CO)C(=O)O]

or a pharmaceutically acceptable salt and/or solvate thereof.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of CIRs useful with the synthetic antigen receptors described herein include, for example, those made with solvents that are pharmaceutically acceptable. Examples of such solvents include water (resulting solvate is called a hydrate) and ethanol and the like. Suitable solvents are physiologically tolerable at the dosage administered.

Methods of making suitable CIRs are described in U.S. patent application Ser. No. 17/249,332.

### Engineered Cells

The synthetic antigen receptor/CIR combination is designed to be used with cells that have been engineered with the synthetic antigen receptor. Accordingly, as used herein, “engineered cells” refers to cells that have been modified to express a synthetic antigen receptor described herein. Any suitable cell type may be used. Engineered cells of a specified type may be described as being derived from the specified cell type, for example engineered immune cells may be described as being derived from immune cells. The cells may be derived from any eukaryote. In some embodiments, additional signaling components may also be introduced into the cell. By way of example, for DAP12-based synthetic antigen receptors, a DAP12 component (e.g. DAP12 (SEQ ID NO: 8 or a functional variant thereof) or a nucleic acid encoding DAP12 (e.g. SEQ ID NO: 7 or a functional variant thereof)) would be introduced into a cell that does not endogenously express DAP12.

In an embodiment, the cells are immune cells, optionally immune cells that can suppress tissue pathology (ex. suppress tumor growth, suppress graft rejection, suppress allergic reactions, suppress autoimmune reactions). In an embodiment, the immune cells are lymphocytes, monocytes, polymorphonuclear cells, erythrocytes or megakaryocytes. In an embodiment, the immune cells are T cells (including ABT cells, GDT cells, MAIT cells, and NKT cells), B cells, NK cells, macrophages, neutrophils, basophils, eosinophils or red blood cell.

The cells may be engineered to express the synthetic antigen receptor within a patient to be treated by introducing a nucleic acid encoding the synthetic antigen receptor into the cell using in vivo delivery tools, such adenovirus, adeno associated virus, retrovirus, lentivirus, synthetic RNA.

In an embodiment, the cells are taken from a human, engineered ex vivo to express the synthetic antigen receptor, and delivered to an unrelated human. In such embodiments, the cells are allogenic. As used herein, the term “allogenic” refers to cells, tissue, DNA, or factors originally obtained (taken or derived) from a subject who is a different individual than the intended recipient of said cells, but who is of the same species as the recipient. Optionally, allogenic cells may be cells from a cell culture. For example, in the context where allogenic transduced or transfected cells are administered to a subject, cells removed from an individual of the same species that is not the subject are transduced or transfected with a vector that directs the expression of a desired component, and the transduced or transfected cells are administered to the subject. In a one embodiment, the cells are allogenic cells obtained from a healthy donor.

In an embodiment, the cells are taken from a human, engineered ex vivo to express the synthetic antigen receptor, and given back to the same human. In such embodiments, the cells are autologous. As used herein, the term “autologous” refers to cells, tissue, DNA or factors originally obtained taken or derived from an individual's own tissues, cells or DNA. For example, in the context where autologous transduced or transfected cells are administered to a subject, cells removed from the subject are transduced or transfected with a vector that directs the expression of a desired component and the transduced or transfected cells are administered to the subject.

The phrase “directs expression” refers to the polynucleotide comprising a sequence that encodes the molecule to be expressed. The polynucleotide may comprise additional sequence that enhances expression of the molecule in question.

The nucleic acid encoding the synthetic antigen receptor may be introduced into the cell by any suitable method known in the art. Suitable methods include but are not limited to transfection, transduction, infection, electroporation, sonoporation, nucleofection, and microinjection. In some embodiments, the nucleic acid construct is introduced into the cell by transfection. Suitable transfection reagents are well known in the art and may include, without limitation, cationic polymers such as polyethylenimine (PEI), cationic lipids such as lipofectamine and related reagents (Invitrogen) and non-liposomal reagents such as Fugene and related reagents (Promega), or calcium phosphate. In some embodiments, the nucleic acid may be introduced into the cells by transduction using a suitable viral vector such as lentivirus, retrovirus, AAV and adenovirus.

The immune cells may be engineered directly within the individual to be treated using nucleic acids formulated for in vivo gene delivery by any suitable transfection method known in the art. Suitable transfection reagents are well known in the art and may include, without limitation, cationic polymers such as polyethylenimine (PEI), cationic lipids such as lipofectamine and related reagents (Invitrogen) and non-liposomal reagents such as Fugene and related reagents (Promega), or calcium phosphate. In some embodiments, the nucleic acid may be introduced into the cells by transduction using a suitable viral vector such as lentivirus, retrovirus, AAV and adenovirus.

To allow for the selection of cells into which the nucleic acid or expression cassette has been introduced, a selectable marker may be introduced into the cell along with the nucleic acid, or along with one or more expression cassettes. The term “selectable marker” as used herein refers to an element in a nucleic acid construct that confers a selective advantage to cells harboring the nucleic acid construct. For example, the selectable marker may encode a protein that is expressed and confers resistance to a specific drug. Alternatively, the selectable marker may encode a protein that is expressed and is essential for cell viability under specific growth conditions. Suitable selectable markers are known to the skilled person. Examples of suitable drug-selectable markers include blasticidin resistance, neomycin resistance, hygromycin resistance, or puromycin resistance.

The selectable marker may be on the same nucleic acid molecule as the expression cassette, or on a different nucleic acid molecule. If the selectable marker is provided on a separate nucleic acid molecule, the nucleic acid molecule with the selectable marker is provided at a lower ratio or percentage than the other nucleic acid molecule(s) e.g. a 1:4 molar ratio, a 1:5 molar ratio, or a 1:10 molar ratio, or e.g. 25%, 20%, or 10% of the total nucleic acids. Cells that take up the selectable marker are likely to have taken up other nucleic acids that are introduced along with the marker, e.g. the desired nucleic acid, thereby allowing for the selection of cells carrying the desired nucleic acid.

The selective pressure applied to the cell will depend on the selective marker present in the nucleic acid construct. As used herein, the term “selective pressure” refers to the growth conditions of the cell that provide a selective advantage in cell viability for a cell harboring the selectable marker. Selective growth conditions may include, without limitation, the addition of a drug or the withdrawal of a component essential for growth. For example, where the selectable marker is an antibiotic resistance gene, selective pressure is applied by the addition of the antibiotic. As another example, where the selectable marker is glutamine synthetase, selective pressure is applied by the withdrawal of glutamine from the growth medium. In another example, the selective agent is methionine sulfoximine (MSX) for selection of glutamine synthetase overexpressing cells. In yet another embodiment, the selective marker is methotrexate for selection of dihydrofolate reductase (DHFR) expressing cells.

### Functionalized Cells

The synthetic antigen receptor/CIR combination (or engineered cell/CIR combination) is designed to generate a functionalized cell comprising a desired target binding domain (TBD) suitable for the intended application or use. As used herein, “functionalized cell” refers to a cell comprising a synthetic antigen receptor (i.e. an engineered cell) that has been covalently modified by a CIR. As will be understood, a functionalized cell therefore comprises the TBD of the CIR used to modify the synthetic antigen receptor.

### Compositions

The synthetic antigen receptors, nucleic acids, engineered or functionalized cells, and/or CIRs for use with the synthetic antigen receptors described herein are suitably formulated in a conventional manner into compositions using one or more carriers or diluents. Accordingly, the present description also includes a composition comprising one or more synthetic antigen receptors, nucleic acids, engineered or functionalized cells, and/or CIRs for use with the synthetic antigen receptors described herein and a carrier or diluent. The synthetic antigen receptors, nucleic acids, engineered or functionalized cells, and/or CIRs for use with the synthetic antigen receptors described herein are suitably formulated into pharmaceutical compositions for administration to subjects in a biologically compatible form suitable for administration in vivo. Accordingly, the present description further includes a pharmaceutical composition comprising the synthetic antigen receptors, nucleic acids, engineered or functionalized cells, and/or CIRs for use with the synthetic antigen receptors described herein, and a pharmaceutically acceptable carrier. In some embodiments the pharmaceutical compositions are used in the treatment of any of the diseases, disorders or conditions described herein.

The synthetic antigen receptors, nucleic acids, engineered or functionalized cells, and/or CIRs for use with the synthetic antigen receptors described herein may be administered to a subject in a variety of forms depending on the selected route of administration, as will be understood by those skilled in the art. For example, the synthetic antigen receptors, nucleic acids, engineered or functionalized cells, and/or CIRs for use with the synthetic antigen receptors described herein may be administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, the CIRs for use with the synthetic antigen receptors, nucleic acids, and/or engineered or functionalized cells described herein are orally administered, for example, with an inert diluent or with an assimilable edible carrier, or it is enclosed in hard or soft shell gelatin capsules, or it is compressed into tablets, or it is incorporated directly with the food of the diet. In some embodiments, the CIR for use with the synthetic antigen receptors, nucleic acids, and/or engineered or functionalized cells described herein is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

In some embodiments, the synthetic antigen receptors, nucleic acids, engineered or functionalized cells, and/or CIRs for use with the synthetic antigen receptors described herein are administered parenterally. For example, solutions of one or more synthetic antigen receptors, nucleic acids, engineered or functionalized cells, and/or CIRs for use with the synthetic antigen receptors described herein are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the synthetic antigen receptors, nucleic acids, engineered or functionalized cells, and/or CIRs for use with the synthetic antigen receptors described herein are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, the synthetic antigen receptors, nucleic acids, engineered or functionalized cells, and/or CIRs for use with the synthetic antigen receptors described herein are formulated for parenteral administration by injection, including using conventional catheterization techniques or infusion. Formulations for injection are, for example, presented in unit dosage form, e.g., in ampoules or in multidose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, synthetic antigen receptors, nucleic acids, engineered or functionalized cells, and/or CIRs for use with the synthetic antigen receptors described herein are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the synthetic antigen receptors, nucleic acids, engineered or functionalized cells, and/or CIRs for use with the synthetic antigen receptors described herein are conveniently delivered in the form of a solution, dry powder formulation or suspension from a pump spray container that is squeezed or pumped by the subject or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound and a suitable powder base such as lactose or starch. The aerosol dosage forms can also take the form of a pump-atomizer.

Compositions suitable for buccal or sublingual administration include tablets, lozenges, and pastilles, wherein a compound is formulated with a carrier such as sugar, acacia, tragacanth, or gelatin and glycerine. Compositions for rectal administration are conveniently in the form of suppositories containing a conventional suppository base such as cocoa butter.

Suppository forms of the synthetic antigen receptors, nucleic acids, engineered or functionalized cells, and/or CIRs for use with the synthetic antigen receptors described herein are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 1^{6t}h Ed., Mack Publishing, Easton, PA, 1980, pp. 1530-1533 for further discussion of suppository dosage forms.

In some embodiments the synthetic antigen receptors, nucleic acids, engineered or functionalized cells, and/or CIRs for use with the synthetic antigen receptors described herein are coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The synthetic antigen receptors, nucleic acids, engineered or functionalized cells, and/or CIRs for use with the synthetic antigen receptors described herein, including pharmaceutically acceptable salts and/or solvates thereof, are suitably used on their own but will generally be administered in the form of a pharmaceutical composition in which the one or more synthetic antigen receptors, nucleic acids, engineered or functionalized cells, and/or CIRs for use with the synthetic antigen receptors described herein (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### Kits

Also provided herein are kits comprising one or more of the synthetic antigen receptors, nucleic acids, and/or engineered or functionalized cells described herein along with a suitable container or packaging and/or instructions for use thereof, such as, for example, for use in the methods or applications described herein. For example the instructions may relate to generating an engineered cell, generating a functionalized cell, treating a disease, disorder or condition treatable by immunotherapy. In an embodiment, the kit further comprises a suitable cognate CIR for use with the synthetic antigen receptor, nucleic acid, and/or engineered cell. In an embodiment, the kit further comprises an applicator or other suitable delivery device. In an embodiment, the kit further comprises one or more additional drugs.

### III. Methods and Uses

As shown herein, functionalized cells comprising a target binding domain can be generated by contacting engineered cells comprising a synthetic antigen receptor described herein (an “engineered cell”) with a suitable covalent immune recruiter (CIR) comprising the target binding domain (TBD) under conditions to allow binding of the donor moiety and acceptor domain and covalent attachment of the covalent binding group to the synthetic antigen receptor, thereby generating a functionalized cell. As will be understood, the suitability of a given TBD will depend on the intended application or use of the functionalized cell. For example, where the intended application of the functionalized cell is for the treatment of a specific cancer, a suitable TBD will be one that binds cells of the specific cancer. The skilled person can readily select a suitable TBD for the intended application or use.

Accordingly, an aspect described herein includes a method for generating a functionalized cell, the method comprising providing a cell comprising a synthetic antigen receptor described herein, and contacting the cell with a suitable cognate CIR (comprising a donor moiety that interacts with the acceptor domain of the synthetic antigen receptor) under suitable conditions to allow binding of the donor moiety and acceptor domain and covalent attachment of the covalent binding group to the synthetic antigen receptor, thereby generating a functionalized cell.

Functionalized cells may be generated for example in vitro, ex vivo, or in vivo. For example, an engineered cell may be contacted in vitro or ex vivo with a suitable cognate CIR. Alternatively, the engineered cell and suitable cognate CIR may be administered separately to a subject, and the functionalized cell generated in vivo.

The functionalized cells described herein may be directed towards a target cell and exhibit a cytotoxic response to the target cell. Accordingly, the functionalized cells described herein are effective tools for directing the functionalized cell to a target cell, and/or triggering a cytotoxic response to the target cell. The target cell may be any desired cell. For example, in the context of cancer therapy, the target cell may be a cancer cell.

Also described herein is a method for recruiting a functionalized cell to a target cell in a subject, comprising administering to the subject an effective amount of a) an engineered cell and a CIR; or b) a functionalized cell.

Further described herein is a method for recruiting a functionalized cell to a target cell in a subject for provoking an immune response to the target cell, comprising administering to the subject an effective amount of a) an engineered cell and a suitable cognate CIR; or b) a functionalized cell comprising a suitable target binding domain (TBD).

Also described herein is a method for targeting a functionalized cell for provoking an immune response to a target cell in a subject, comprising administering to the subject an effective amount of a) an engineered cell and a suitable cognate CIR; or b) a functionalized cell comprising a suitable TBD.

Also described herein is a method for provoking cellular phagocytosis of a target cell in a subject, comprising administering to the subject an effective amount of a) an engineered cell and a suitable cognate CIR; or b) a functionalized cell comprising a suitable TBD.

Also described herein are methods of: recruiting an immune cell for immunotherapy, recruiting an immune cell for provoking an immune response to a target cell, targeting an immune cell for provoking an immune response to a target cell, and provoking cellular phagocytosis of a target cell.

Further described herein is a use of the synthetic antigen receptors, nucleic acids, engineered or functionalized cells, and/or CIRs for use with the synthetic antigen receptors described herein for the preparation of a medicament for the methods and uses described herein, for example the treatment of a disease, disorder, or condition. Such diseases, disorders, or conditions that are treatable by immunotherapy include, without limitation, cancer, autoimmune diseases, and allergy, and transplant rejection.

Also described herein is a method of treating a disease, disorder or condition that is treatable by provoking an immune response, comprising administering to a subject in need thereof a therapeutically effective amount of a) an engineered cell and a suitable cognate CIR; or b) a functionalized cell comprising a suitable TBD. Such diseases, disorders, or conditions that are treatable by immunotherapy include, without limitation, cancer, autoimmune diseases, and allergy, and transplant rejection.

Also described herein is a use of a) an engineered cell and a suitable cognate CIR; or b) a functionalized cell comprising a suitable TBD for treating a disease, disorder or condition treatable by immunotherapy. An aspect also includes use of a) an engineered cell and a suitable cognate CIR; or b) a functionalized cell comprising a suitable TBD for the preparation of a medicament for treating of a disease, disorder or condition treatable by immunotherapy. Such diseases, disorders, or conditions that are treatable by immunotherapy include, without limitation, cancer, autoimmune diseases, and allergy, and transplant rejection.

In an embodiment, the disease, disorder or condition treatable by immunotherapy is cancer, therefore the present disclosure includes a method of treating cancer comprising administering to a subject in need thereof a therapeutically effective amount of a) an engineered cell and a suitable cognate CIR (comprising a target binding domain (TBD) that binds the cancer being treated); or b) a functionalized cell. The present disclosure also includes a use of a) an engineered cell and a suitable cognate CIR; or b) a functionalized cell comprising a suitable TBD for treatment of cancer as well as a use of a) an engineered cell and a suitable cognate CIR comprising a suitable TBD; or b) a functionalized cell comprising a suitable TBD for the preparation of a medicament for treatment of cancer. The disclosure further includes a) an engineered cell and a suitable cognate CIR; or b) a functionalized cell comprising a suitable TBD for use in treating cancer. In an embodiment, the functionalized cell comprising a suitable TBD is administered for the prevention of cancer in a subject such as a mammal having a predisposition for cancer.

In an embodiment, the cancer is one that is impacted or treatable by immunotherapy. In an embodiment, the cancer is one that is impacted or treatable by activation of immune cells. In an embodiment, the cancer is one that is impacted or treatable by provoking an immune response to tumor cells. In an embodiment, the cancer is one that is impacted or treatable by provoking phagocytosis of tumor cells.

In an embodiment, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In an embodiment, the cancer is selected from prostate cancer, breast cancer, ovarian cancer and glioblastoma. In an embodiment, the cancer is prostate cancer. In another embodiment, the cancer is glioblastoma.

In further embodiments, the present disclosure also includes a method of treating a disease, disorder or condition treatable by immunotherapy, comprising administering to a subject in need thereof a therapeutically effective amount of a) an engineered cell and a suitable cognate CIR; or b) a functionalized cell comprising a suitable TBD, in combination with another agent useful for treatment of the disease, disorder or condition treatable by immunotherapy. The present disclosure also includes a use of a) an engineered cell and a suitable cognate CIR; or b) a functionalized cell comprising a suitable TBD, in combination with an agent useful for treatment of a disease, disorder or condition treatable by immunotherapy, for treatment of such disease, disorder or condition.

In a further embodiment, the disease, disorder or condition treatable by immunotherapy is cancer and the a) engineered cell and a suitable cognate CIR; or b) functionalized cell comprising a suitable TBD, are administered in combination with one or more additional cancer treatments. In another embodiment, the additional cancer treatment is selected from radiotherapy, chemotherapy, targeted therapies such as antibody therapies and small molecule therapies such as tyrosine-kinase and serine-threonine kinase inhibitors, immunotherapy, hormonal therapy and anti-angiogenic therapies.

Effective amounts vary according to factors such as the disease state, age, sex and/or weight of the subject. Accordingly, the amount of a given synthetic antigen receptor, nucleic acid, engineered or functionalized cell, and/or CIR for use with the synthetic antigen receptors described herein that will correspond to an effective amount will vary depending upon factors, such as the given synthetic antigen receptor, nucleic acid, engineered or functionalized cell, and/or CIR for use with the synthetic antigen receptors described herein, the pharmaceutical formulation, the route of administration, the type of condition, disease or disorder, the identity of the subject being treated, and the like, but can nevertheless be routinely determined by one skilled in the art.

Suitable administration schedules may include, without limitation, at least once a week, from about one time per two weeks, three weeks or one month, about one time per week to about once daily, 2, 3, 4, 5 or 6 times daily. The length of the treatment period may depend on a variety of factors, such as the severity of the disease, disorder or condition, the age of the subject, the concentration and/or the activity of the synthetic antigen receptors, nucleic acids, engineered or functionalized cells, and/or CIRs for use with the synthetic antigen receptors described herein and/or a combination thereof. It will also be appreciated that the effective dosage of the synthetic antigen receptors, nucleic acids, engineered or functionalized cells, and/or CIRs for use with the synthetic antigen receptors described herein used for the treatment may increase or decrease over the course of a particular treatment regime. Changes in dosage may result and become apparent by standard diagnostic assays known in the art. In some instances, chronic administration is required. For example, the synthetic antigen receptors, nucleic acids, engineered or functionalized cells, and/or CIRs for use with the synthetic antigen receptors described herein are administered to the subject in an amount and for duration sufficient to treat the subject.

In an embodiment, the subject is a mammal. In another embodiment, the subject is human.

The synthetic antigen receptors, nucleic acids, engineered or functionalized cells, and/or CIRs for use with the synthetic antigen receptors described herein may be either used alone or in combination with other known agents useful for treating diseases, disorders or conditions as defined above. When used in combination with other agents useful in treating such diseases, disorders or conditions, the synthetic antigen receptors, nucleic acids, engineered or functionalized cells, and/or CIRs for use with the synthetic antigen receptors described herein may be administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. In other embodiments, the combination of agents is administered to a subject in a non-contemporaneous fashion. In an embodiment, the engineered cell, functionalized cell, composition, etc. described herein is administered with another therapeutic agent simultaneously or sequentially in separate unit dosage forms or together in a single unit dosage form. Accordingly, the present description provides a single unit dosage form comprising the engineered cell, functionalized cell, composition, etc. described herein, an additional therapeutic agent, and a pharmaceutically acceptable carrier.

The dosage of the synthetic antigen receptors, nucleic acids, engineered or functionalized cells, and/or CIRs for use with the synthetic antigen receptors described herein varies depending on many factors such as the pharmacodynamic properties of the compound/cell, the mode of administration, the age, health and weight of the recipient, the nature and extent of the symptoms, the frequency of the treatment and the type of concurrent treatment, if any, and the clearance rate of the compound/cell in the subject to be treated. One of skill in the art can determine the appropriate dosage based on the above factors. In some embodiments, synthetic antigen receptors, nucleic acids, engineered or functionalized cells, and/or CIRs for use with the synthetic antigen receptors described herein is administered initially in a suitable dosage that is adjusted as required, depending on the clinical response. Dosages will generally be selected to maintain sufficient levels of the engineered cell, functionalized cell, composition, etc. described herein.

### EXAMPLES

The following non-limiting examples are illustrative of the present disclosure.

### Example 1: Covalent Binding of Molecular Adapters to the DNP-Specific TAC is Essential for Activating Conventional ABT Cells

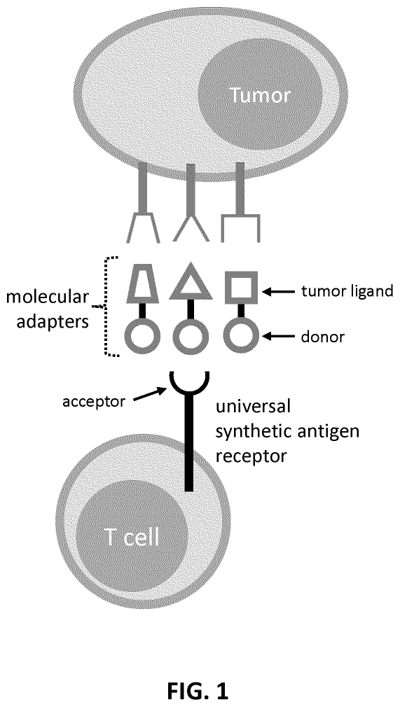

A universal synthetic antigen receptor employs an acceptor domain (typically a single-chain antibody or scFv) that binds a molecular adapter that links the T cell to the tumor (FIG. 1). The adapters comprise a donor moiety that binds the acceptor domain, and a tumor binding domain (FIG. 1). By creating a library of adapters, a T cell engineered with a single universal synthetic antigen receptor can be directed against multiple tumor targets. This strategy offers the potential for both tailoring the T cells to the antigenic repertoire of the tumor and enabling multispecific targeting through the administration of multiple adapter molecules, each specific for a different tumor antigen, that bind the same universal receptor. Covalent immune recruiters (CIRs; FIG. 2A)²⁶ leverage the benefits of bifunctional small molecules and covalent drugs to chemically link immune receptors to cancer cells. CIRs consist of: (i) a donor moiety that will form a covalent bond with an immune receptor, typically an antibody, (ii) a covalent binding group that mediates the covalent binding and (iii) a ligand for a molecule on the surface of tumor cells (FIG. 2A). CIRs establish covalent bonding via a chemically reactive component in the donor moiety and nucleophilic proximal amino acids in the binding site of the immune receptor yielding an immune receptor that is permanently re-directed towards the tumor target (FIG. 2B). High selectivity and fast reaction kinetics of CIRs directed to anti-dinitrophenyl (DNP) antibodies has been demonstrated previously, even in the presence of human serum which contains an abundance of off-target proteins²⁶.

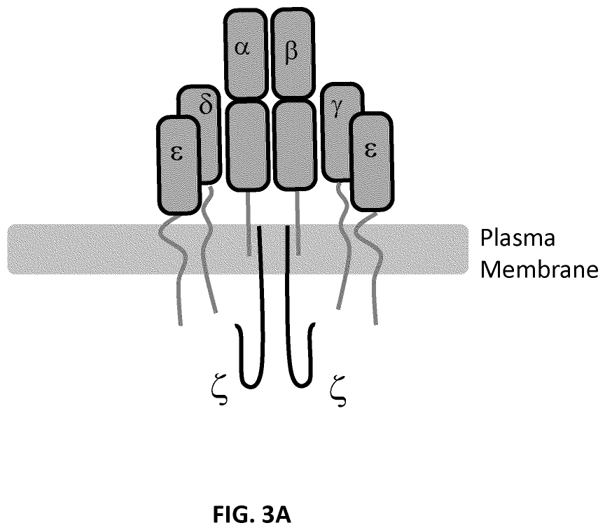

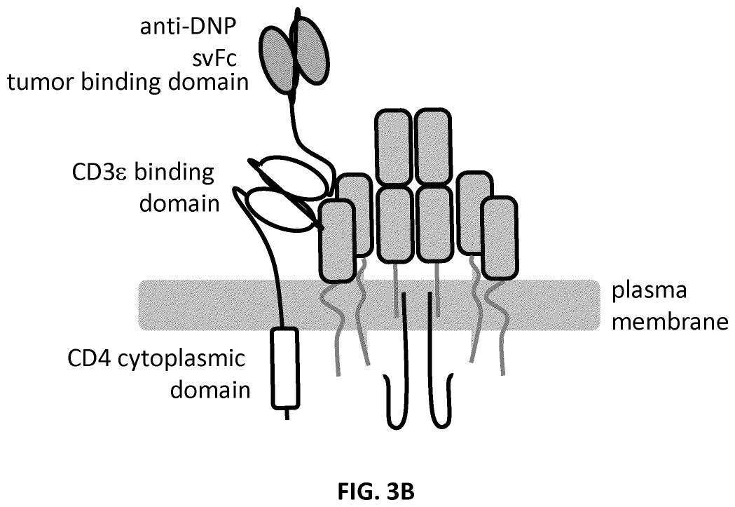

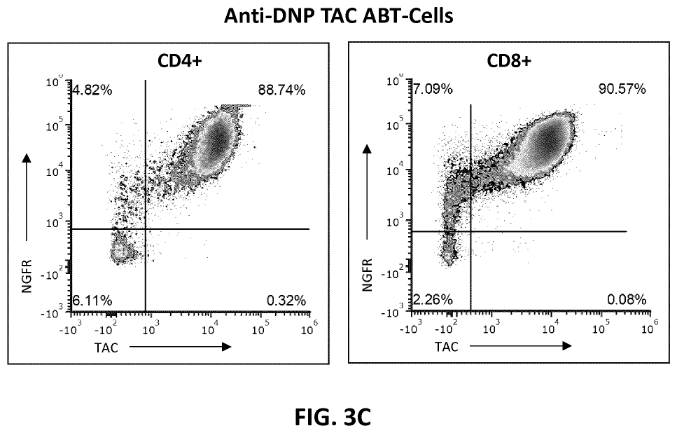

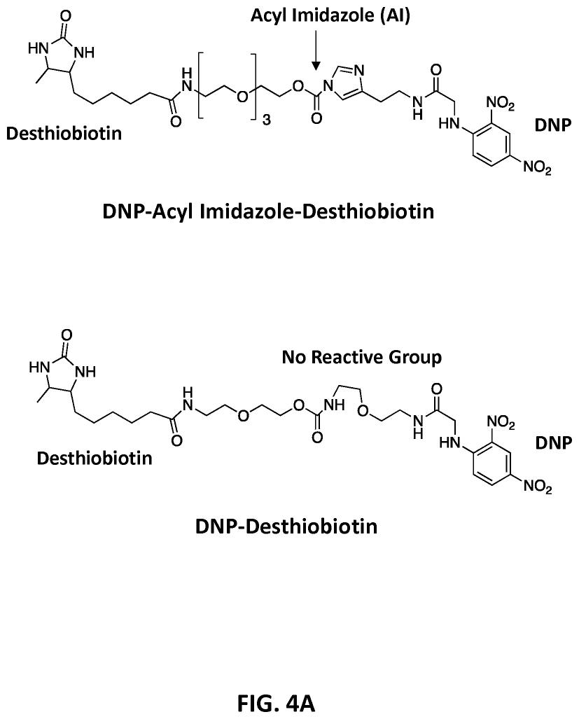

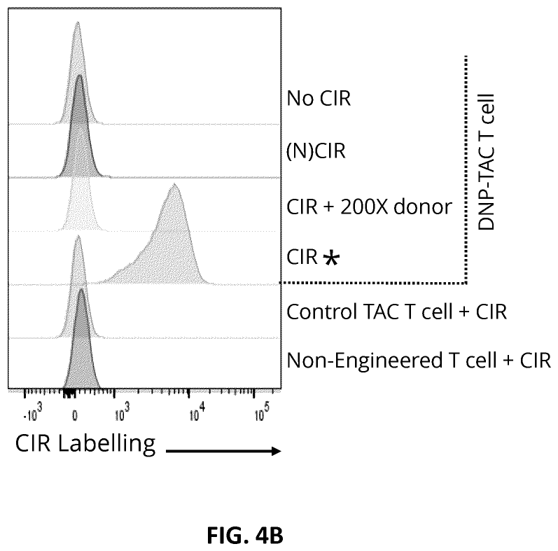

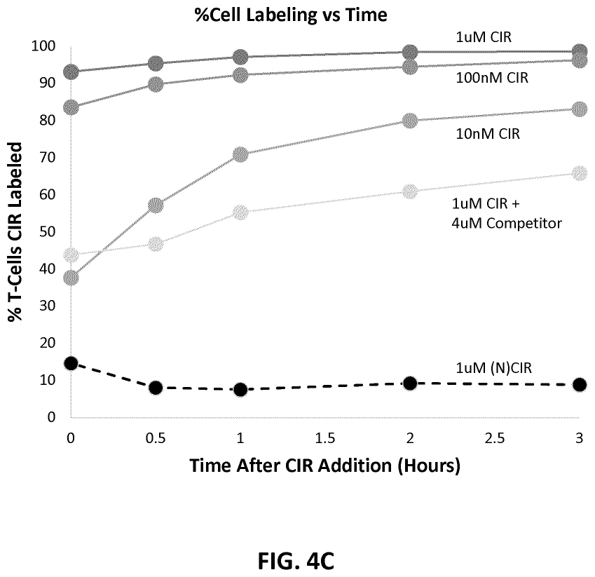

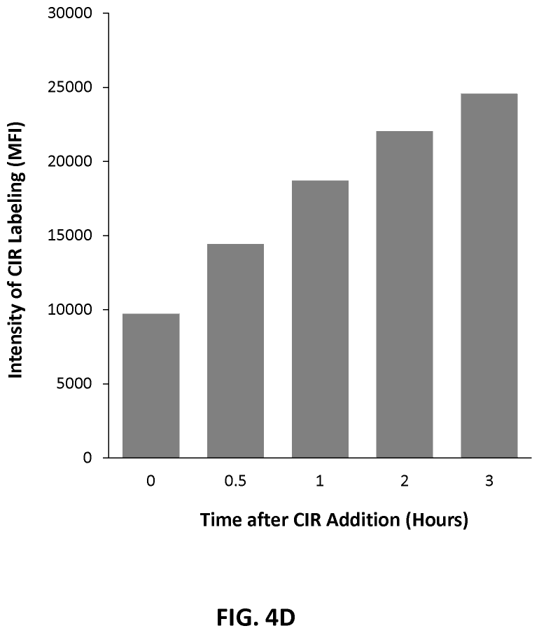

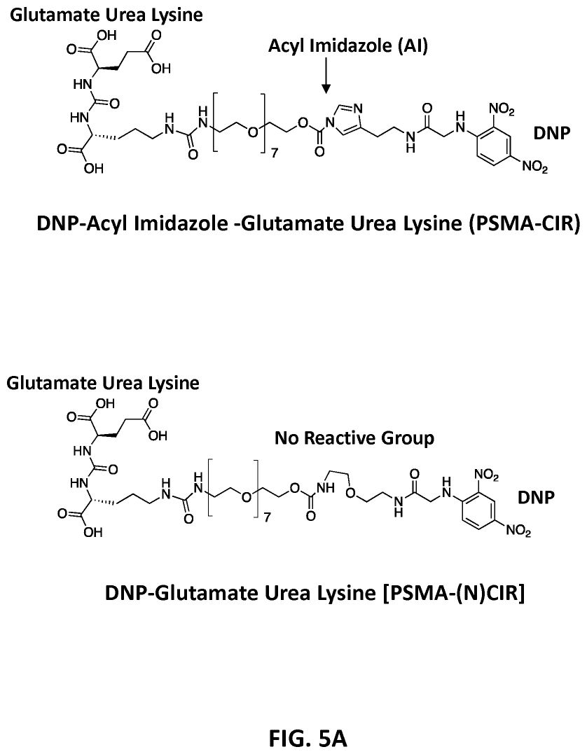

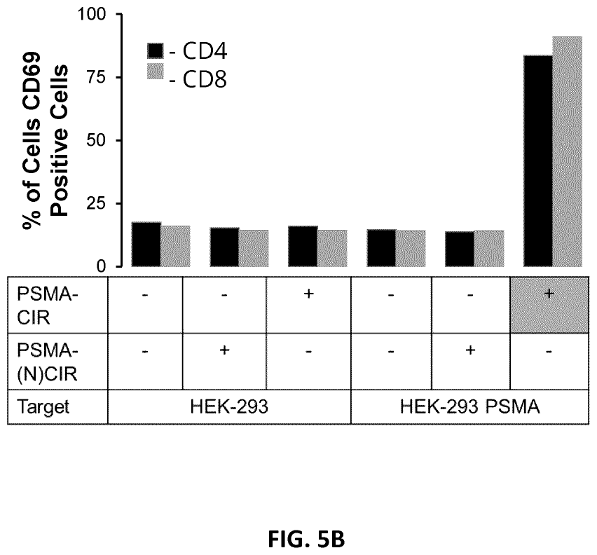

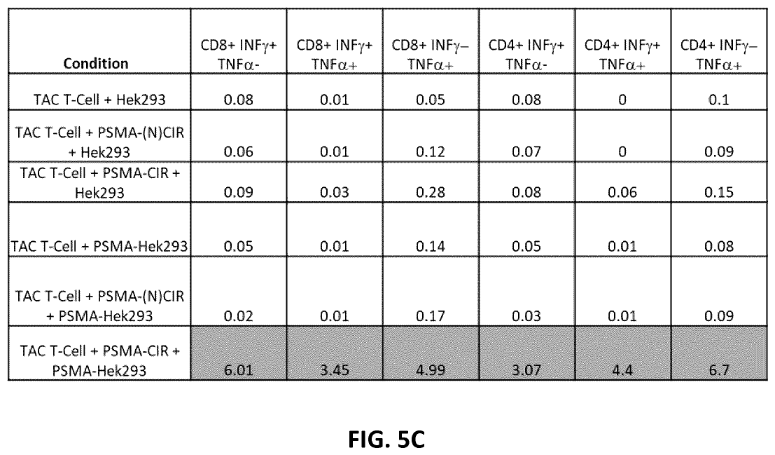

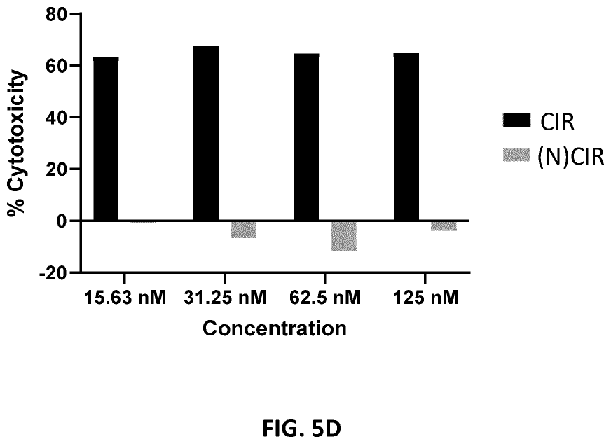

A T cell antigen coupler (TAC) receptor was designed to bind dinitrophenol (DNP) using an scFv derived from the monoclonal antibody, SPE7, with the orientation of heavy variable chain (VH)-light variable chain (VL) (scFv: SEQ ID NOs: 1 and 2) (FIG. 3B). T cells engineered with the DNP-specific TAC (anti-DNP TAC: SEQ ID NOs: 17 and 18) (DNP-TAC T cells) display high levels of surface expression of the receptor (FIG. 3C). Anti-DNP TAC T cells (DNP-TAC T cells) bound DNP-based CIRs (FIG. 4A) with high selectivity under conditions where comparable adapters that cannot form a covalent linkage [(N)CIR]failed to bind (FIG. 4B). The CIRs covalently link the DNP-TAC T cells with very rapid kinetics (FIG. 4C) but maximal loading of DNP-TAC T cells requires several hours (FIG. 4D). To demonstrate tumor-specific programing of DNP-TAC T cells, a DNP-CIR was employed that carried glutamate urea, a ligand for the tumor target, PSMA (PSMA-CIR; FIG. 5A). As a control for covalent binding, a non-covalent control [PSMA-(N)CIR; FIG. 5A] was included. DNP-TAC T cells loaded with PSMA-CIR demonstrated PSMA-specific activation based on upregulation of CD69 (FIG. 5B) and production of IFN-gamma and TNF-alpha (FIG. 5C). In contrast, DNP-TAC T cells loaded with PSMA-(N)CIR failed to demonstrated any evidence of activation in the presence of PSMA-expressing target cells (FIG. 5B/5C). Likewise, when DNP-TAC T cells were co-cultured with PSMA-positive LNCaP cells in the presence of PSMA-CIR or PSMA-(N)CIR, only the combination of the DNP-TAC T cells and the DNP-glutamate urea-CIR could trigger killing of PSMA-expressing LNCaP prostate cancer cells whereas the analogous (N)CIR was unable to direct T cell cytotoxicity (FIG. 5D). It should be noted that the 125 nM (N)CIR was ineffective at engaging the DNP-TAC T cells to kill LNCaP cells whereas the CIR was effective at engaging tumor killing at concentrations as low as 15 nM CIR (FIG. 5D). These results establish the importance of covalent chemistry in activating DNP-TAC-engineered T cells via molecular adapters.

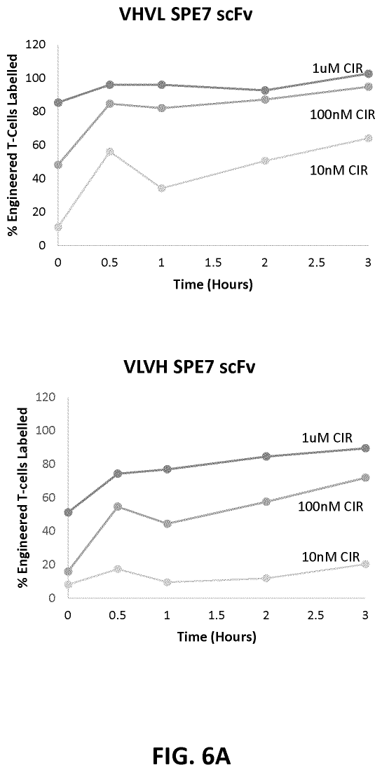

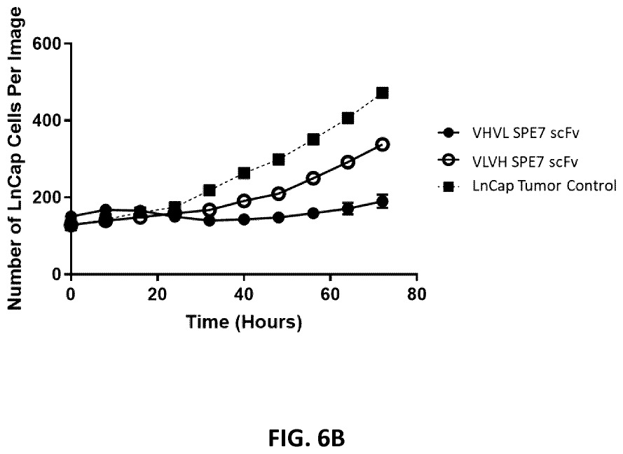

Since the orientation of the heavy and light regions within the scFv can affect performance, an alternate DNP-specific TAC was generated where the SPE7 scFv was generated in the light variable chain (VL)-heavy variable chain (VH) orientation (scFv: SEQ ID NOs: 3 and 4; TAC: SEQ ID NO: 19 and 20). Time course of DNP-Acyl Imidazole-Desthiobiotin loading of T cells engineered with DNP-specific TAC receptors revealed higher loading of the T cells engineered with the original version of the DNP-TAC with the SPE7 scFv in the VH-VL orientation (FIG. 6A). Similar, assessment of LNCaP killing in the presence of the PSMA-CIR also demonstrated improved killing by T cells engineered with the original version of the DNP-TAC with the SPE7 scFv in the VH-VL orientation (FIG. 6B).

### Experimental Details:

T cell engineering: Peripheral blood mononuclear cells (PBMCs) were isolated from a donor's blood. The PBMC's were plated in a 96 well plate with 1E5 cells per well. To each well Immunocult CD3/CD28/CD2 soluble activator, hlL-2 (1.5 ng/mL), and hlL-7 (10 ng/mi) were added for preferential expansion of ABT-cells. Two days after activation anti-DNP TAC gamma retrovirus was spun into a pre-coated retronectin 24 well plate. 10 wells of activated T-cells were combined and added to the same virus coated well in the 24 well plate and incubated overnight to allow for transduction. From here the T-cells were scaled according to growth and maintained at a concentration of 1E6 cells/ml. On D14 the cells were phenotyped via flow cytometry. The cells were stained with protein-L Biotin, streptavidin PE, anti-NGFR FITC, anti-CD4 Pacific Blue, and anti-CD8 Alexa fluor 700. The cells were fixed and run on a flow cytometer to visualize transduction. The transduction can be observed in FIG. 3C and is ~89% and ~90% for the CD4 and CD8 positive T-cells respectively.

Labeling with CIR/(N)CIR: ABT-cells were labelled overnight at 4 degrees in T-cell media with 1 uM of either DNP-Acyl Imidazole-Desthiobiotin or DNP-Desthiobiotin. DNP-Glycine competitor was also added to some conditions with at 200 uM as a competitor to assess labelling specificity. After the overnight incubation the samples were washed 3 times with FACs buffer to remove any non-covalently bound molecule and stained with Streptavidin-PE to assess labelling. The samples were also stained with near IR Live/Dead, anti-CD4 Pacific Blue, and anti-CD8 Alexa fluor 700 to allow for appropriate gating on live ABT-cells. The samples were assessed for Streptavidin-PE labelling via flow cytometry.

For labeling time-course studies, T-cells were incubated with varying concentrations (10 nM, 100 nM, 1 uM) of either DNP-Acyl Imidazole-Desthiobiotin or DNP-Desthiobiotin for different time periods up to 3 hours at room temperature in FACs buffer. The “0” hour time point was the final time point and accounts for the 5-minute period needed to spike in CIR/(N)CIR and spin down to stop labelling. After adding DNP-Acyl Imidazole-Desthiobiotin or DNP-Desthiobiotin to all time points the cells were washed 3 times and stained for flow cytometry analysis as described above.

Measuring CD69 following stimulation with HEK-293 cells: In a 96 well round bottom plate, 2E5 HEK-293 or HEK-293 PSMA cells were incubated with 2E5 DNP-TAC T-cells that were pre labelled for 6 hours with either PSMA-CIR, PSMA-(N)CIR or media alone. The cells were co-cultured together overnight at 37 degrees. To stop the activation, 20 mM EDTA was added and incubated for 15 minutes. The cells were spun down, media was aspirated, and the cells were stored in 200 μL of cRPMI at 4 degrees prior to staining. The cells were stained with Live-Dead Zombie Aqua, anti-CD3 BV605, anti-CD4 Pacific Blue, anti-CD8 Alexa Fluor 700, anti-NGFR Viobright FITC, and anti-CD69 BV650. Upon staining the cells were run on the Beckman Coulter Cytoflex flow cytometer and analyzed for CD69 expression.

Measuring cytokine production following stimulation with HEK-293 cells: In a 96 well round bottom plate, 4E5 HEK-293 or HEK-293 PSMA were incubated with 4E5 Anti-DNP TAC ABT-cells that were prelabelled for 6 hours with either PSMA-CIR, PSMA-(N)CIR or media alone. The cells were co-cultured together for 4 hours at 37 degrees. Golgi plug was added prior to the co-incubation to prevent cytokine secretion. To stop the activation 20 mM EDTA was added and incubated for 15 minutes. The cells were spun down, media was aspirated, and the cells were stored in 200 μL of cRPMI overnight at 4 degrees. The next day, the cells were surface stained with anti-CD4 Pacific Blue, anti-CD8 Alexa Fluor 700, and anti-NGFR Viobright FITC. The cells were then fixed and permeabilized using a fix/perm buffer solution. The cells were then intracellularly stained with anti-CD3 BV605, anti-TNF-alpha PE-CY7, and anti-IFN-gamma APC. Cells were then run on the Beckman Coulter Cytoflex flow cytometer. The percent of cells positive for TNF-alpha and IFN-gamma, as well as cells positive for both were recorded in the table for the separate CD4 and CD8 T-cell populations.

Measuring T cell mediated killing of LNCaP cells: LNCaP prostate cancer cells, which are prostate specific membrane antigen (PSMA) positive, were engineered with Nuclight Red lentivirus to produce cells with nuclear localized red fluorescence to allow for ease of imaging in these experiments. In this experiment Nuclight Red LNCaP cells were plated at 7000 cells per well in a 96 well flatbottom plate overnight. The next day Anti-DNP TAC ABT-cells were added to the tumor cells at an effector to target ration of 8:1. Also added to the wells were PSMA-CIR or PSMA-(N)CIR at a range of concentrations from 15.63 nM to 125 nM. The 3 components were co-cultured for 3 days with 4 images per well taken every 6 hours. Samples were run in triplicate. The number of LnCap cells per well at each time point was calculated using the Incucyte cell by cell analysis software and plotted. The area under the growth curve was used as a metric for tumor cell growth for this figure. The larger the area, the greater the tumor cell growth that occurred over the 3 days. The area under the curve for the LnCap control and each condition were used to calculate the % cytotoxicity.

### Example 2. Covalent Binding of Molecular Adapters to the DNP-Specific Synthetic Antigen Receptors is Essential for Activating Non-Canonical GDT Cells

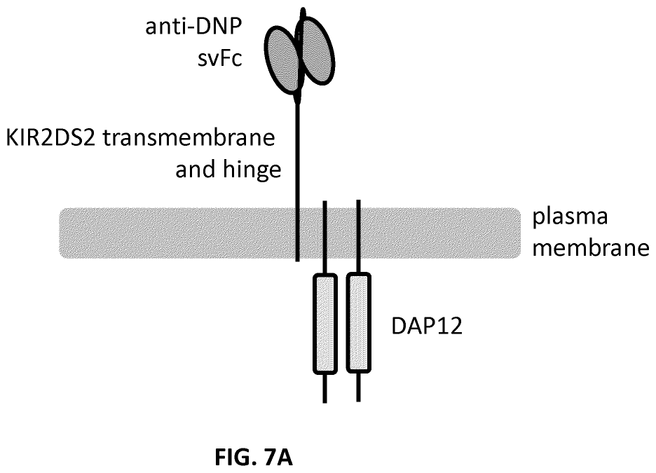

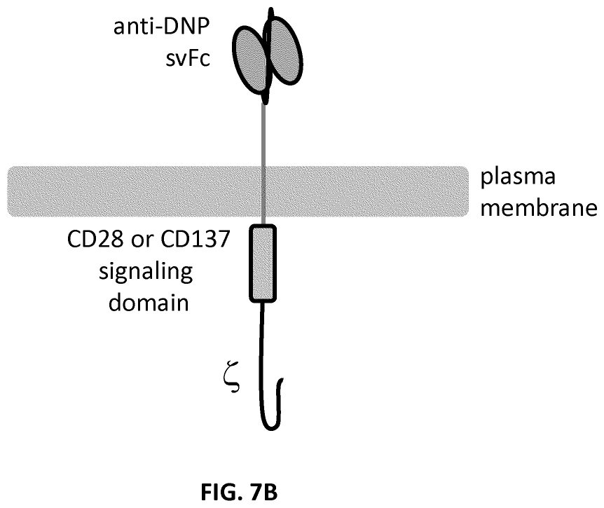

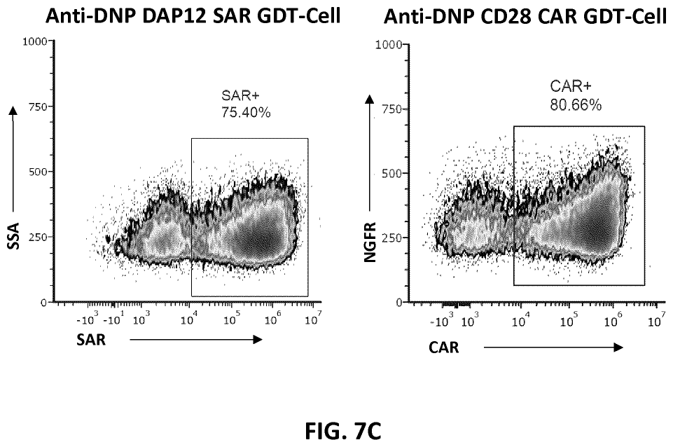

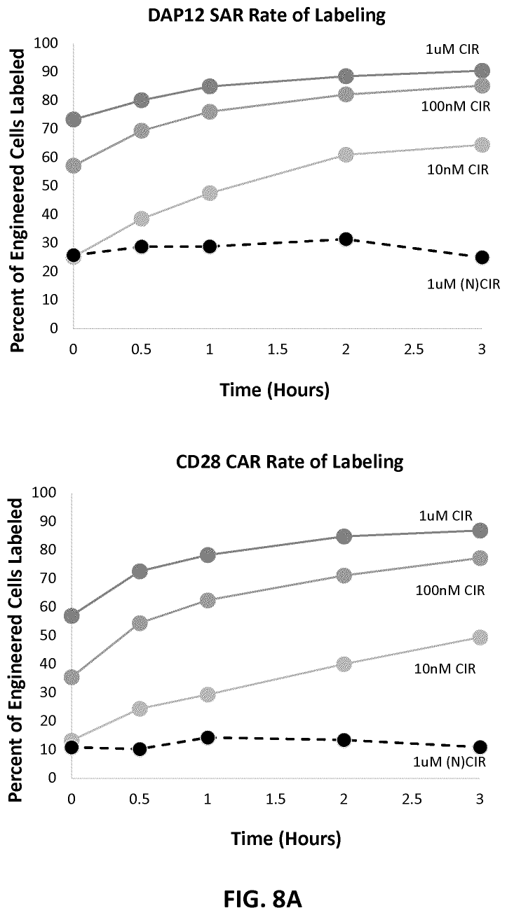

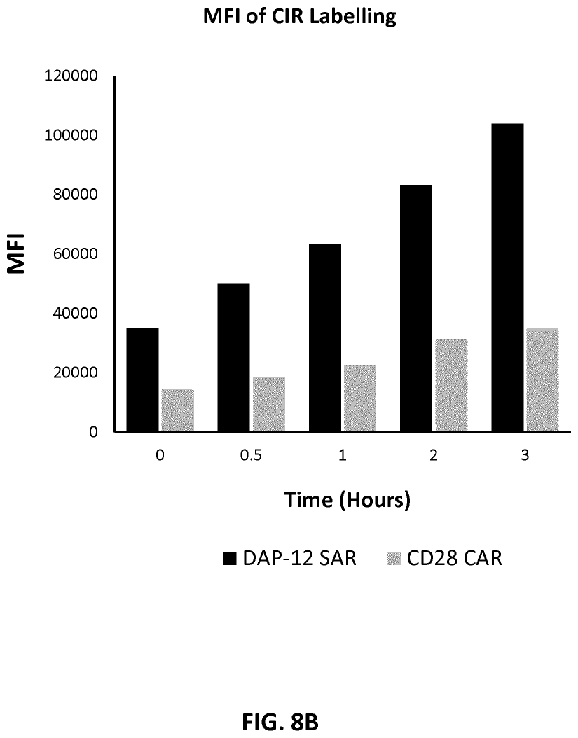

Universal synthetic antigen receptors were designed based on other contemporary scaffolds, either a DAP12-based synthetic antigen receptor (DAP12-SAR; SEQ ID NOs: 21 and 22; FIG. 7A) or a CD28-based chimeric antigen receptor (CD28 CAR; SEQ ID NOs: 25 and 26; FIG. 7B). The DNP-specific scFv derived from SPE7 in the VH-VL orientation was introduced into each synthetic antigen receptor to enable binding of DNP-based CIRs. Both synthetic antigen receptors were engineered into non-canonical GDT cells and display excellent surface expression (FIG. 7C). Similar to the outcomes with the Anti-DNP TAC T cells described in Example 1, GDT cells engineered with either DNP-specific synthetic antigen receptor were loaded efficiently and rapidly with covalent molecular adapters but non-covalent molecular adapters failed to load the T cells to a significant degree (FIG. 8A). The T cells engineered with the DAP12-SAR were loaded with more CIR than the T cells engineered with the CD28 CAR (FIG. 8B).

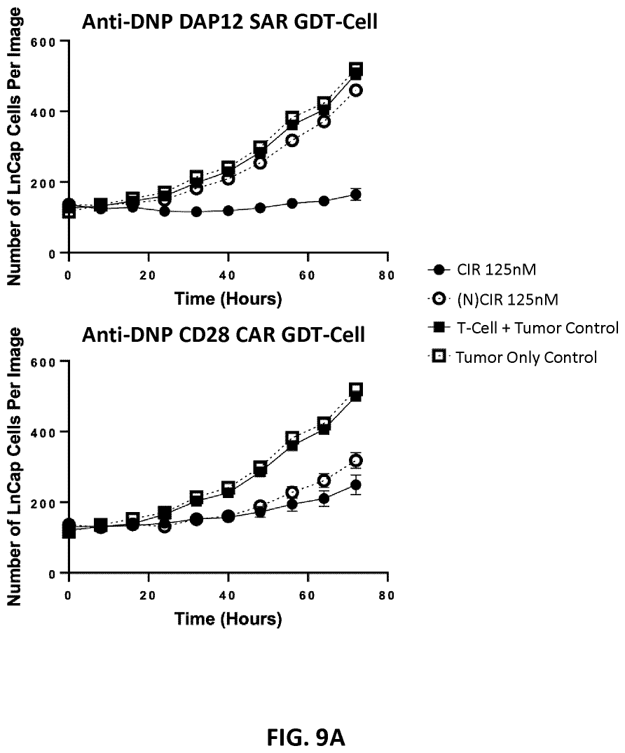

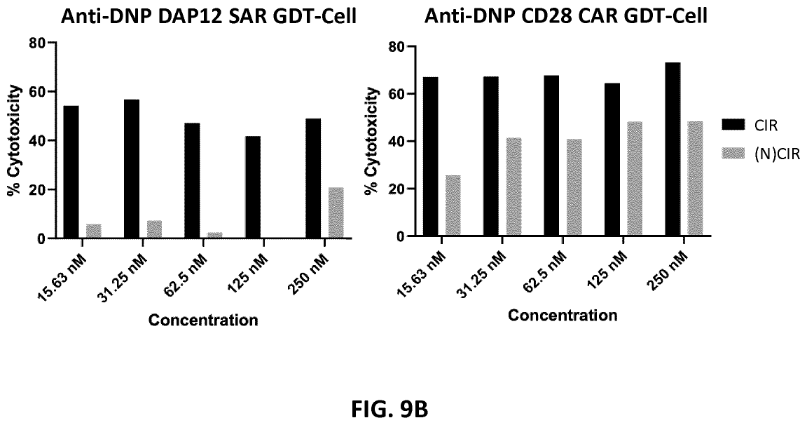

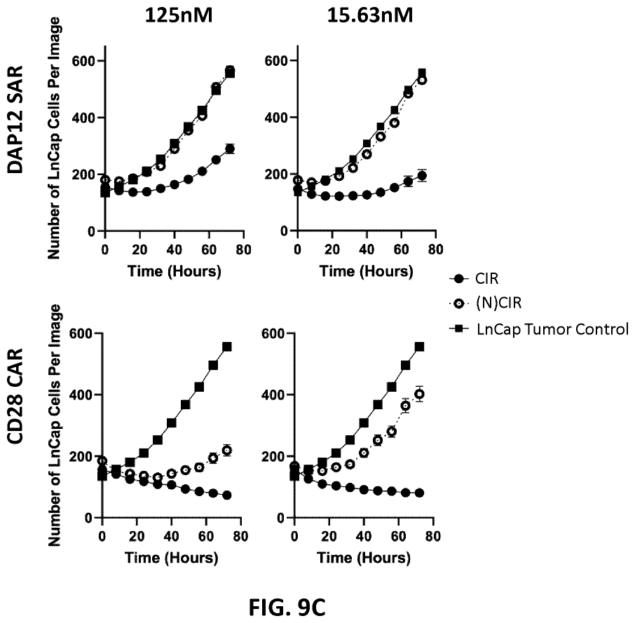

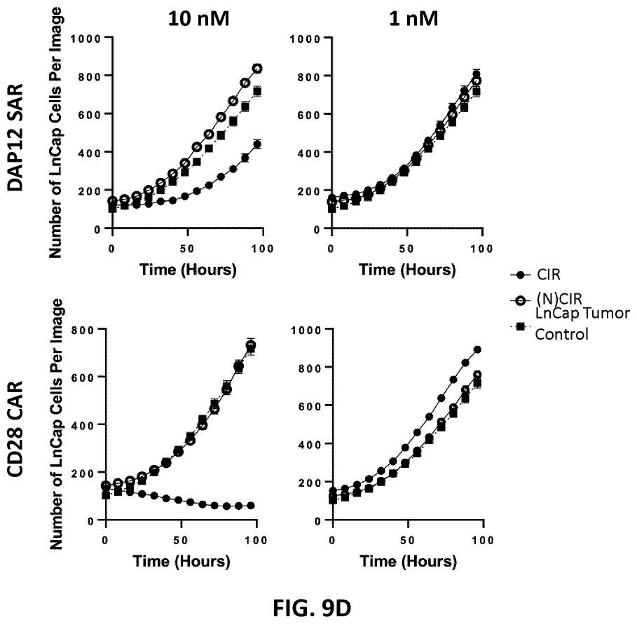

When Anti-DNP DAP12-SAR GDT cells were co-cultured with PSMA-positive LNCaP cells in the presence of a high concentration (125 nM) of PSMA-CIR or PSMA-(N)CIR, only the combination of the Anti-DNP DAP12-SAR GDT cells and the PSMA-CIR could trigger killing of PSMA-expressing LNCaP prostate cancer cells whereas the analogous (N)CIR was unable to direct T cell cytotoxicity (FIG. 9A), which confirms the importance of covalent binding in activating the DAP12-SAR through the molecular adapter. In contrast, when Anti-DNP CD28 CAR GDT cells were co-cultured with PSMA-positive LNCaP cells in the presence of 125 nM PSMA-CIR or PSMA-(N)CIR both molecular adapters could direct T cell cytotoxicity (FIG. 9A). In a subsequent experiment, the performance of the two engineered populations was evaluated over a range of PSMA-CIR/(N)CIR concentrations (15.6 nM-250 nM). The ability of the PSMA-CIR to produce LNCaP killing from both Anti-DNP DAP12-SAR GDT cells and Anti-DNP CD28 CAR GDT cells was sustained across all concentrations whereas killing diminished with lower concentrations of the PSMA-(N)CIR demonstrating that covalent binding is critical at limiting concentrations of the molecular adapter (FIG. 9B/9C). Further, the PSMA-CIR retained the ability to trigger robust LNCaP killing by CD28 CAR T cells at very low concentrations (10 nM) where the PSMA-(N)CIR was unable to elicit any killing at 10 nM, reaffirming the importance of covalent binding of the molecular adapter at limiting concentrations (FIG. 9D).

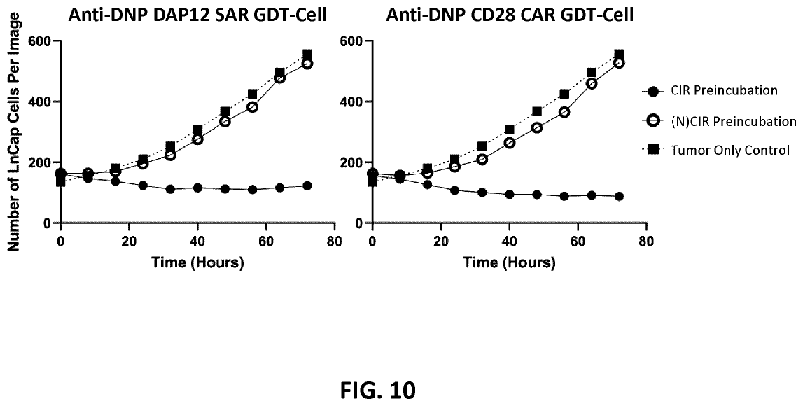

Experiments where the engineered GDT cells were loaded with 1 uM PSMA-CIR or PSMA-(N)CIR and washed prior to co-culture with LNCaP cells (FIG. 10) were also performed. In this case, covalent binding was absolutely critical for tumor cell killing by T cells engineered with either the Anti-DNP DAP12-SAR or the Anti-DNP CD28 CAR.

### Experimental Details:

Engineered GDT-cells: PBMCs were seeded at 4E6 cells per well in a 24 well G-Rex plate in the presence of 1 ug/ml zoledronate and 1.5 ng/ml human IL2. Three days later, the cells were incubated overnight in a 24 well non tissue culture treated plate that was pre coated with retronectin and retrovirus supernatant containing viruses encoding either the Anti-DNP DAP12-SAR or the Anti-DNP CD28 CAR. The cells were then transferred back to the G-Rex plate and cultured for a further 11 days at which point, the cell product was collected, CD4/CD8 T cells were removed by magnetic selection and the purified engineered GDT cells were cryopreserved.

Flow cytometry: To assess the fraction of GDT cells which express the Anti-DNP synthetic antigen receptor, the cells were stained with the following antibodies/fluorophores: Myc biotin, streptavidin PE, vδ2 APC, TCR ab FITC, and CD3 BV605. The CD3 BV605, TCR ab FITC, and Vδ2 APC were used to specifically gate on Vδ2 GDT-cells. The anti-Myc biotin and streptavidin PE secondary were used to assess transduction level.

For labeling time-course studies, T-cells were incubated with varying concentrations (10 nM, 100 nM, 1 uM) of either DNP-Acyl Imidazole-Desthiobiotin or DNP-Desthiobiotin for different time periods up to 3 hours at room temperature in FACs buffer. The “0” hour time point was the final time point and accounts for the 5-minute period needed to spike in CIR/(N)CIR and spin down to stop labelling. After adding DNP-Acyl Imidazole-Desthiobiotin or DNP-Desthiobiotin to all time points the cells were washed 3 times and stained for flow cytometry analysis as described above.

Measuring T cell mediated killing of LNCaP cells: LNCaP prostate cancer cells, which are prostate specific membrane antigen (PSMA) positive, were engineered with Nuclight Red lentivirus to produce cells with nuclear localized red fluorescence to allow for ease of imaging in these experiments. In this experiment Nuclight Red LNCaP cells were plated at 7000 cells per well in a 96 well flatbottom plate overnight. The next day anti-DNP GDT-cells were added to the tumor cells at an effector to target ration of 4:1. Also added to the wells were PSMA-CIR or PSMA-(N)CIR at a range of concentrations from 1 nM to 250 nM, depending upon the experiment. The 3 components were co-cultured for 3 days with 9 images per well taken every 8 hours. Samples were run in triplicate. The number of LNCaP cells per well at each time point was calculated using the Incucyte cell by cell analysis software and plotted. The area under the growth curve was used as a metric for tumor cell growth for this figure. The larger the area, the greater the tumor cell growth that occurred over the 3 days. The area under the curve for the LNCaP control and each condition were used to calculate the % cytotoxicity.

### Example 3. SuFEx Chemistry can be Used to Produce CIRs that Selectively Activate GDT Cells Engineered with DNP-Specific Universal Synthetic Antigen Receptors

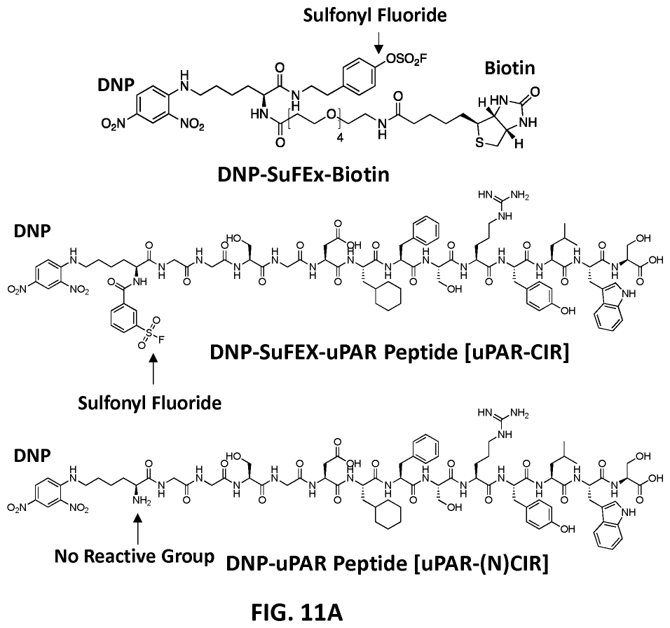

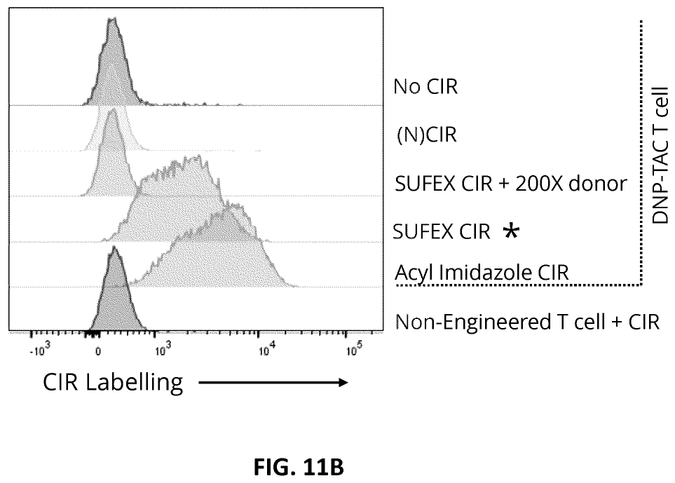

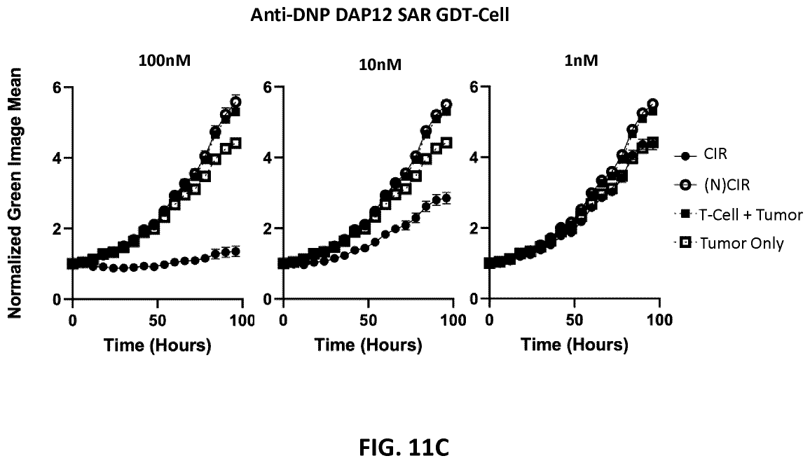

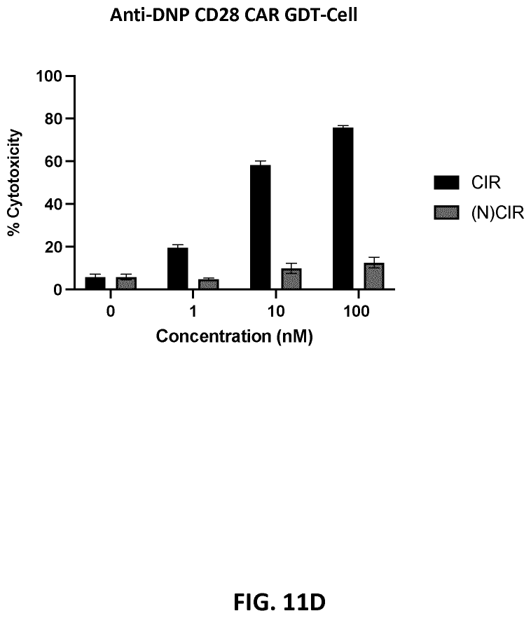

The previous examples employed acyl imidazole chemistry to serve as the covalent binding group for proximity labeling of the anti-DNP scFv. To demonstrate that other chemistries can be used for this purpose, CIRs employing SuFEx chemistry were designed for proximity labeling. Here, DNP-SuFEx-Desthiobiotin was synthesized for labeling studies and DNP-SuFEx-uPAR binding peptide (uPAR-CIR) was synthesized to serve as a molecular adapter that will mediate T cell activation in response to uPAR-expressing cells (FIG. 11A); as a control for covalent binding, DNP-uPAR binding peptide [uPAR-(N)CIR]was synthesized (FIG. 11A). Similar to the observation with acyl imidazole CIRs, the SuFEx CIR could bind Anti-DNP TAC ABT cells under conditions where the (N)CIR could not (FIG. 11B). Importantly, only the uPAR-CIR could mediate lysis of uPAR-expressing A172 cells when co-cultured with Anti-DNP DAP12-SAR GDT cells whereas the uPAR-(N)CIR was unable to mediate lysis of the A172 cells when co-cultured with Anti-DNP DAP12-SAR GDT cells (FIG. 11C). Similarly, Anti-DNP CD28 CAR GDT cells only exhibited cytotoxicity towards A172 cells when co-cultured with the uPAR-CIR, and not when co-cultured with the uPAR-(N)CIR (FIG. 11D).

### Experimental Details:

Engineering ABT cells with Anti-DNP TAC: Peripheral blood mononuclear cells (PBMCs) were isolated from a donor's blood. The PBMC's were plated in a 96 well plate with 1E5 cells per well. To each well Immunocult CD3/CD28/CD2 soluble activator, hlL-2 (1.5 ng/mL), and hlL-7 (10 ng/ml) were added for preferential expansion of ABT-cells. Two days after activation anti-DNP TAC gamma retrovirus was spun into a pre-coated retronectin 24 well plate. 10 wells of activated T-cells were combined and added to the same virus coated well in the 24 well plate and incubated overnight to allow for transduction. From here the T-cells were scaled according to growth and maintained at a concentration of 1E6 cells/ml. On D14 the cells were phenotyped via flow cytometry. The cells were stained with protein-L Biotin, streptavidin PE, anti-NGFR FITC, anti-CD4 Pacific Blue, and anti-CD8 Alexa fluor 700. The cells were fixed and run on a flow cytometer to visualize transduction. The transduction can be observed in this figure and is ~89% and ~90% for the CD4 and CD8 positive T-cells respectively.

Labeling with CIR/(N)CIR: ABT-cells were labelled overnight at 4 degrees in T-cell media with 1 uM of either DNP-SuFEx-Desthiobiotin, DNP-Acyl Imidazole-Desthiobiotin or DNP-Desthiobiotin. DNP-Glycine competitor was also added to some conditions with at 200 uM as a competitor to assess labelling specificity. After the overnight incubation the samples were washed 3 times with FACs buffer to remove any non-covalently bound molecule and stained with Streptavidin-PE to assess labelling. The samples were also stained with near IR Live/Dead, anti-CD4 Pacific Blue, and anti-CD8 Alexa fluor 700 to allow for appropriate gating on live αβ T-cells. The samples were assessed for Streptavidin-PE labelling via flow cytometry.

Engineering GDT cells with Anti-DNP DAP12-SAR or Anti-DNP CD28 CAR: PBMC were seeded at 4E6 cells per well in a 24 well G-Rex plate in the presence of 1 ug/ml zoledronate and 1.5 ng/ml human IL2. Three days later, the cells were incubated overnight in a 24 well non tissue culture treated plate that was pre coated with retronectin and retrovirus supernatant containing viruses encoding the Anti-DNP DAP12-SAR or Anti-DNP CD28 CAR. The cells were then transferred back to the G-Rex plate and cultured for a further 11 days at which point, the cell product was collected, CD4/CD8 T cells were removed by magnetic selection and the purified engineered GDT cells were cryopreserved.

Measuring T cell mediated killing of A172 cells: A172 cells, which are urokinase type plasminogen activator receptor (uPAR) positive, were engineered with eGFP lentivirus to produce green fluorescent protein expressing cells. In this experiment eGFP A172 cells were plated at 5000 cells per well in a 96 well flatbottom plate overnight. The next day Anti-DNP DAP12-SAR or Anti-DNP CD28 CAR GDT-cells were added to the tumor cells at an effector to target ration of 8:1. Also added to the wells were uPAR-CIR or uPAR-(N)CIR at a range of concentrations from 1 nM, 10 nM and 100 nM. The 3 components were co-cultured for 3 days with 9 images per well taken every 8 hours. Samples were run in triplicate. The green image mean of the wells at each time point was calculated using the Incucyte cell by cell analysis software and plotted to yield the following figures. The values were normalized to the starting green image mean. A172 tumor cells alone were also included as a control. For the analysis of killing by Anti-DNP CD28 CAR GDT cells, the area under the growth curve (AUC) was analyzed using PRISM Graphpad and to calculate the % cytotoxicity. Percent cytotoxicity was calculated as:% Cytotoxicity=((AUC Tumor Alone-AUC Sample)/(AUC Tumor Alone))×100%.

### Example 4. SuFEx Chemistry can be Used to Produce CIRs that Functionally Redirect ABT Cells Engineered with DNP-Specific Universal Synthetic Antigen Receptors

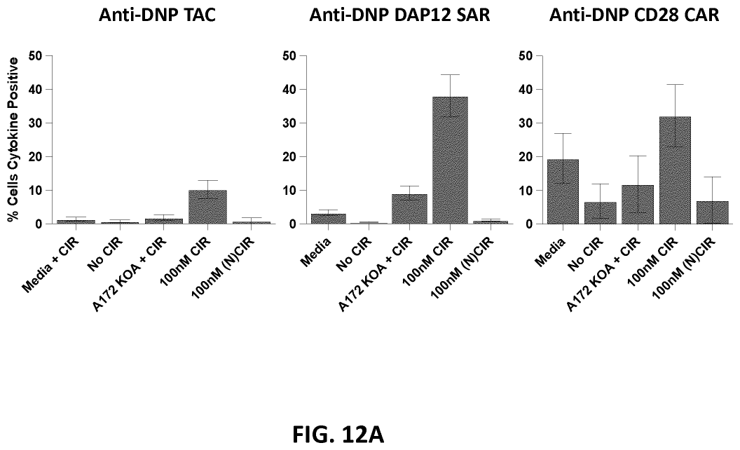

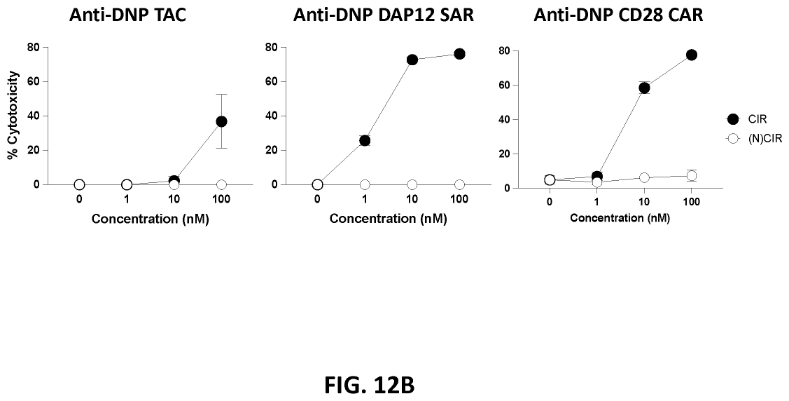

The previous Example employed the SuFEX uPAR targeting CIR to covalently label and redirect anti-DNP scFv containing DAP12 SAR and CD28 CAR engineered GDT cells toward uPAR expressing cancer cells. The present Example demonstrates that this CIR can also be utilized with ABT cells engineered with the same Anti-DNP TAC, Anti-DNP DAP12 SAR, and Anti-DNP CD28 CAR synthetic receptors. Inclusion of the uPAR-CIR in the co-cultures elicited cytokine production in all engineered T cells (FIG. 12A). This cytokine production did not occur when T cells were co-cultured with A172KOA cells, which lack uPAR, and the uPAR-CIR, which confirms target-dependent activation of the T cells by the CIR. In contrast, no cytokine production above background (media control) was observed when the T cells were co-cultured with A172 cells in the presence of the uPAR-(N)CIR, confirming the importance of the covalent attachment. Both CD4 and CD8 T-cell populations showed the same results (FIG. 12A). Likewise, when Anti-DNP TAC, Anti-DNP DAP12 SAR, and Anti-DNP CD28 CAR T cells were co-cultured with uPAR-positive A172 cells in the presence of increasing concentrations of uPAR-CIR or uPAR-(N)CIR, only the combination of the engineered T cells and the uPAR-CIR could trigger killing of the A172 glioblastoma cancer cells whereas the analogous (N)CIR was unable to direct T cell cytotoxicity above background (FIG. 12B). It should be noted that the uPAR-CIR was effective over a range of concentrations from 1 nM to 100 nM whereas the uPAR-(N)CIR was ineffective at all concentrations. These results establish the importance of an alternative covalent chemistry in activating multiple receptor types which all utilize different initial signalling pathways to activate the T cell. This work further shows that in the context of SuFEx chemistry, the same CIR/SAR combinations work equally well with GDT cells and ABT cells.

### Experimental Details:

Measuring cytokine production following stimulation with A172 cells: In a 96 well round bottom plate, 4E5 A172 cells (uPAR expressing) or A172KOA cells (a version of A172 where uPAR was removed through CRISPR/Cas9) were incubated with 4E5 Anti-DNP TAC, Anti-DNP DAP12 SAR, or Anti-DNP CD28 CAR ABT-cells. The cells were co-cultured together for 4 hours at 37 degrees with either media or 100 nM of uPAR-CIR or uPAR-(N)CIR and Golgi plug to prevent cytokine secretion. To stop the activation, 20 mM EDTA was added and incubated for 15 minutes. Cells were stained for surface expression of CD4 and CD8, fixed and permeabilized in Cytofix/Cytoperm buffer, and stained with APC-conjugated mouse anti-human IFN-y, and FITC-conjugated mouse anti-human TNF-a. Cells were then run on the Beckman Coulter Cytoflex flow cytometer. The percent of cells positive for TNF-a and IFN-y for CD4 and CD8 T-cell populations were graphed separately showing the same overall trends.

Measuring ABT cell mediated killing of A172 cells: A172 cells, which are uPAR positive, were engineered with eGFP lentivirus to produce green fluorescent protein expressing cells. In this experiment eGFP A172 cells were plated at 5000 cells per well in a 96 well flatbottom plate overnight. The next day Anti-DNP TAC, Anti-DNP DAP12-SAR, or Anti-DNP CD28 CAR ABT cells were added to the tumor cells at an effector to target ration of 8:1. Also added to the wells were uPAR-CIR or uPAR-(N)CIR at concentrations of 1 nM, 10 nM and 100 nM. The 3 components were co-cultured for 4 days with 9 images per well taken every 8 hours. Samples were run in triplicate. A172 tumor cells alone were also included as a control. The green image mean of the wells at each time point was calculated using the Incucyte S3's analysis software. The values were normalized to the starting green image mean for each well. The area under the normalized green image mean growth curve (AUC) was analyzed using PRISM Graphpad and used to calculate the % cytotoxicity. Percent cytotoxicity was calculated as:% Cytotoxicity=((AUC Tumor Alone-AUC Sample)/(AUC Tumor Alone))×100%.

### Example 5. ABT Cells Engineered with DNP-Specific Universal Synthetic Antigen Receptors Show Enhanced Activation in the Presence of CIR Compared to (N)CIR

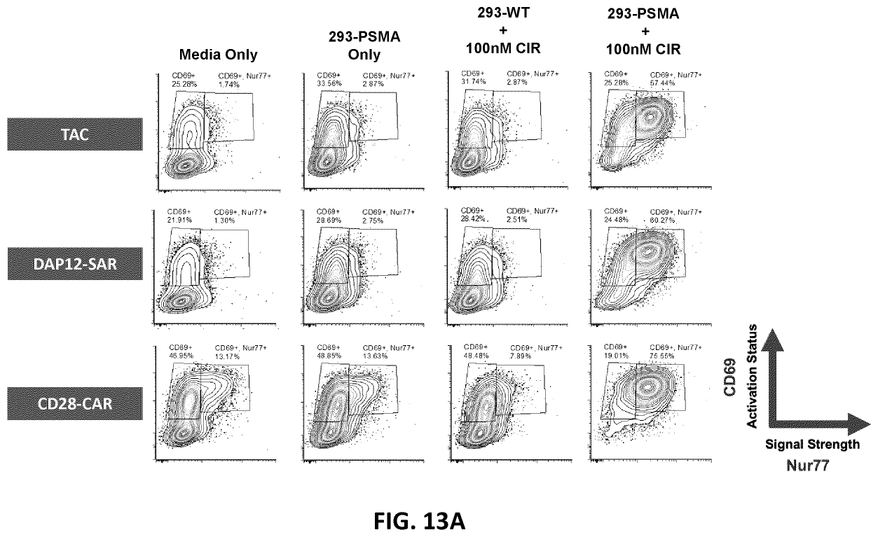

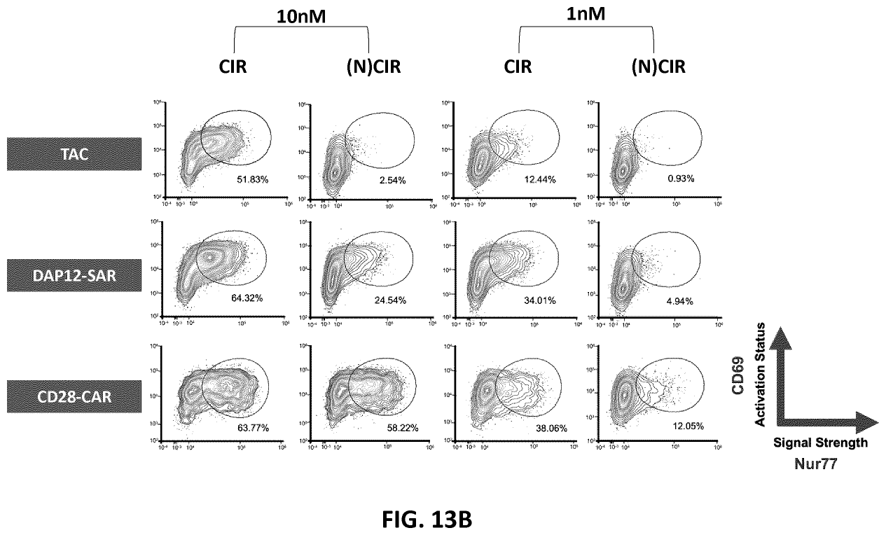

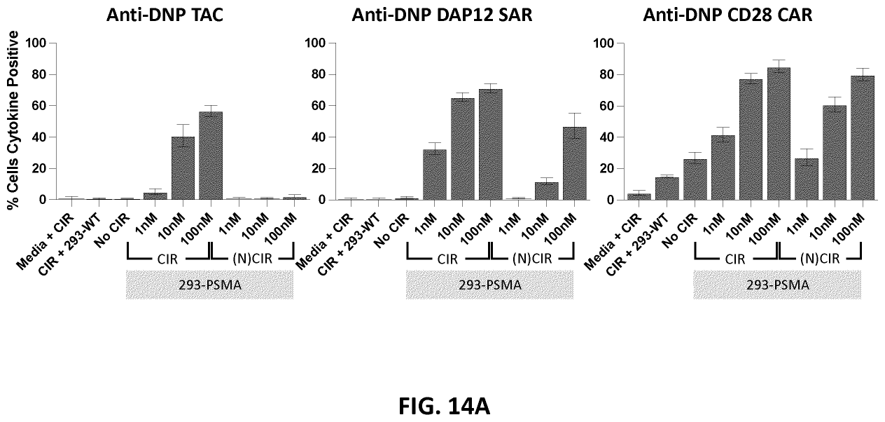

T cell activation is a function of the strength of signal delivered through the activating receptor. As each of the TAC, DAP12 SAR, and CD28 CAR will activate ABT cells through different mechanisms, surrogate activation markers were examined following stimulation via CIR or (N)CIR. For this work, Nur77 expression was used as a measure of integrated signal intensity from the synthetic antigen receptor and CD69 was used as a marker of early T cell activation. First, both the basal and activation-induced levels of Nur77 and CD69 were characterized on the engineered T cells. Here, ABT cells were engineered with either Anti-DNP TAC (TAC), Anti-DNP DAP12 SAR (DAP12-SAR), or Anti-DNP CD28 CAR (CD28-CAR). The engineered T cells were subsequently co-cultured with either 293-WT, that lack PSMA, or 293-PSMA, that express PSMA, in the presence/absence of PSMA-CIR or PSMA-(N)CIR (FIG. 5A). (FIG. 13A). Engineered T cells were co-cultured with 293-WT or 293-PSMA in the presence 100 nM PSMA-CIR. As controls, T cells were cultured in medium alone or co-culture with 293-PSMA cells alone. Differences were observed in basal activation level of the ABT cells engineered with the three different receptors. The CD28 CAR shows the highest basal level of activation while the TAC and DAP12 SAR little basal activation. Upregulation of Nur77 and CD69 was only observed when the engineered ABT cells were co-cultured with 293-PSMA and 100 nM PSMA-CIR (CIR). No off-target T cell activation was observed as the combination of 293-WT and 100 nM PSMA-CIR produced no increase of Nur77 or CD69 above the media-only control. (FIG. 13B). To characterize the importance of covalency on Nur77 and CD69 upregulation, the engineered ABT cells were co-cultured with 293-PSMA cells in the presence of PSMA-CIR (CIR) and PSMA-(N)CIR (NCIR) at 10 nM and 1 nM concentration. For all engineered T cells, upregulation of Nur77 and CD69 was stronger with CIR compared to (N)CIR. Receptor biology did influence the dependency on covalent attachment. At 10 nM CIR/(N)CIR, Anti-DNP-CD28 CAR T cells displayed similar activation whereas at 1M, the PSMA-CIR produced much greater activation of the T cells compared to the PSMA-(N)CIR. In the context of the Anti-DNP DAP12-SAR T cells, the PSMA-CIR elicited greater activation at both concentrations. Finally, with regard to the Anti-DNP TAC-T cells, only the PSMA-CIR could elicit T cell activation. This work reinforces the utility of the CIRs for activating ABT cells through multiple synthetic receptors.

### Experimental Details:

Measuring CD69 and Nur77 Expression of Activated ABT cells: 5E5 engineered T cells and 5E5 293-PSMA cells, that express PSMA, or 293-WT cells, that do not express PSMA, were incubated for 4 hours at 37° C. and 5% CO2 with varying concentrations of PSMA-CIR, PSMA-(N)CIR (1 nM, 10 nM, and 100 nM), or media alone. Cells were stained for surface marker expression via Pacific Blue-conjugated mouse anti-human CD4, and AlexaFluor700-conjugated mouse anti-human CD8a, VioBright FITC-conjugated mouse anti-human NGFR, and anti-huCD69, fixed and permeabilized in Permiabilization concentrate diluted in permeabilization diluent, and stained intracellularly with PE-conjugated mouse anti-mouse Nur77. Flow cytometry data were acquired and analyzed as displayed.

### Example 6. CIR Mediated Reprogramming of all ABT Cells Engineered with DNP-Specific Universal Synthetic Antigen Receptors Show Enhanced Functionalization when Compared to (N)CIR

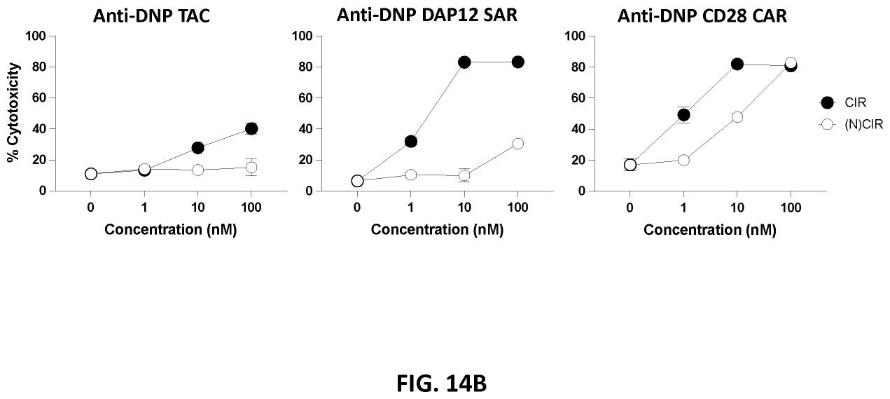

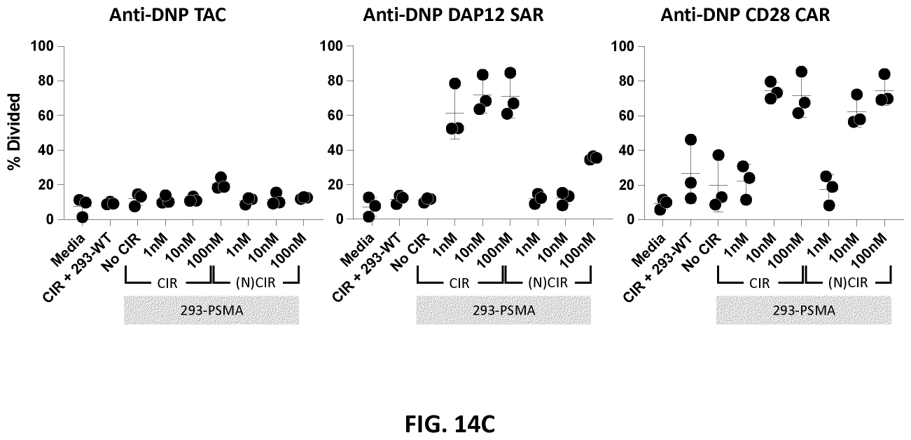

Here, ABT cells were engineered with either Anti-DNP TAC (TAC), Anti-DNP DAP12 SAR (DAP12-SAR), and Anti-DNP CD28 CAR (CD28-CAR). To demonstrate tumor-specific programing of these engineered cells, PSMA-CIR (FIG. 5A) was utilized, and compared to the PSMA-(N)CIR (FIG. 5A) as a non-covalent control. All three of the engineered receptors when reprogrammed with the PSMA-CIR exhibited target specific production of interferon-gamma and tumor necrosis factor-alpha over a range of CIR concentrations when co-cultured with 293-PSMA cells (FIG. 14A shows the total cytokine producing cells). TAC T cells were critically-dependent upon covalent attachment and displayed no cytokine production when co-cultured with 293-PSMA cells in the presence of PSMA-(N)CIR. Similar results were observed with DAP12-SAR T cells, however some cytokine production was observed with higher concentrations of PSMA-(N)CIR but the cytokine production did not reach the level of PSMA-CIR. In fact, the highest concentration of PSMA-(N)CIR (100 nM) stimulated similar cytokine production as the lowest concentration of PSMA-CIR (1 nM), which suggests that the CIR is 100-times more potent than the (N)CIR in this setting. The CD28-CAR T cells displayed equivalent levels of cytokine production at the highest concentration of both adapters, while the PSMA-CIR elicited increased cytokine production at lower concentrations (1 nM, 10 nM). As controls, T cells were cultured with PSMA-CIR in the absence of tumor cells tumor cells (Media+CIR) or with 293-PSMA cells without CIR (No CIR), or with 293-WT cells in the presence of CIR (293-WT+CIR); the controls showed no cytokine production indicating that activation requires both the molecular adapter (CIR) and the target (PSMA). The capacity of PSMA-CIR and PSMA-(N)CIR to elicit cytotoxicity against PSMA-positive LNCaP prostate tumor cells was evaluated. (FIG. 14B). Similar to the results for cytokine production (FIG. 14A), cytotoxicity was critically dependent upon covalent attachment of the adapter to the TAC-engineered and DAP12 SAR-engineered T cells. No killing was observed with TAC T cells cultured in the presence of (N)CIR. Again, cytotoxicity by Anti-DNP DAP12 SAR T cells in the presence of 100 nM PSMA-(N)CIR was comparable to the cytotoxicity observed with 1 nM PSMA-CIR indicating a 100-fold increase in potency of the CIR. In the case of Anti-DNP CD28 CAR T cells, cytotoxicity was comparable at 100 nM of PSMA-CIR and PSMA-(N)CIR, but the PSMA-CIR elicited greater cytotoxicity at lower concentration (1 nM, 10 nM); cytotoxicity of Anti-DNP CD28 CAR T cells in the presence of 1 nM PSMA-CIR was comparable to cytotoxicity in the presence of 10 nM PSMA-(N)CIR indicated a 10-fold increase in potency provided by the CIR. To assess whether the activation induced by CIR could provide a stimulus sufficient to promote T cell proliferation, the proliferative response of the engineered ABT cells was assessed following exposure to 293-PSMA cells in the presence of PSMA-CIR or PSMA-(N)CIR (FIG. 14C). The Anti-DNP TAC T cells only entered proliferation when co-cultured with 293-PSMA cells and 100 nM PSMA-CIR; in all other conditions, Anti-DNP TAC T cells failed to proliferate above background. Anti-DNP DAP12 SAR T cells displayed robust proliferation in response to 293-PSMA cells when co-cultured with PSMA-CIR at any concentration (1 nM, 10 nM, or 100 nM). In contrast, Anti-DNP12 SAR T cells did not proliferate in response to 293-PSMA cells in the presence of 1 nM or 10 nM PSMA-(N)CIR and only displayed modest proliferation in the presence of 293-PSMA cells and 100 nM PSMA-(N)CIR, which is consistent with the prior functional readout that indicated the PSMA-CIR was 100-times more potent than the PSMA-(N)CIR in the ability to stimulate T cell function via the Anti-DNP DAP12 SAR. Similar to the results in FIG. 14A, the PSMA-CIR performed similarly to the PSMA-(N)CIR when included in co-cultures of Anti-DNP CD28 CAR T cells and 293-PSMA cells. As controls, T cells were cultured with PSMA-CIR and 293-WT cells, which lack PSMA, (CIR+293) or with 293-PSMA cells without CIR (293-PSMA). Anti-DNP TAC T cells and Anti-DNP DAP12 SAR T cells displayed no increased proliferation in the control conditions relative to T cells cultured in medium alone (Media). Anti-DNP CD28 CAR T cells displayed increased proliferation in response to CIR+293 and 293-PSMA indicating some degree of non-specific activation due to the presence of 293 cells. Overall, these data confirm the importance of covalent attachment of the molecular adapter to the synthetic antigen receptor to elicit functional attributes of the engineered T cells (ie. Cytokine production, cytotoxicity and proliferation). These data also reveal that the receptor biology has a pronounced influence on the requirement for covalent attachment to elicit T cell function.

### Experimental Details:

Measuring cytokine production following stimulation with HEK-293 cells: In a 96 well round bottom plate, 4E5 HEK293-PSMA cells (PSMA expressing) or HEK293-WT cells were incubated with 4E5 Anti-DNP TAC ABT cells, Anti-DNP DAP12 SAR ABT cells, or Anti-CD28 CAR ABT-cells. The cells were co-cultured together for 4 hours at 37 degrees with either media or 100 nM of PSMA-CIR or PSMA-(N)CIR and Golgi plug to prevent cytokine secretion. To stop the activation 20 mM EDTA was added and incubated for 15 minutes. Cells were stained for surface expression of CD4 and CD8, fixed and permeabilized in Cytofix/Cytoperm buffer, and stained with APC-conjugated mouse anti-human interferon-gamma, and FITC-conjugated mouse anti-human tumor necrosis factor-alpha. Cells were then run on the Beckman Coulter Cytoflex flow cytometer. The percent of cells positive for tumor necrosis factor-alpha and interferon-gamma for CD4 and CD8 T-cell populations were graphed separately showing the same overall trends.

Measuring ABT cell mediated killing of LNCaP cells: LNCaP cells, which express PSMA, were engineered with Nuclight Red lentivirus to produce red nuclear fluorescent protein expressing cells. In this experiment Nuclight Red LNCaP cells were plated at 5000 cells per well in a 96 well flatbottom plate overnight. The next day Anti-DNP TAC ABT cells, Anti-DAP12-SAR ABT cells, or Anti-DNP CD28 CAR ABT cells were added to the tumor cells at an effector to target ration of 8:1. Also added to the wells were PSMA-CIR or PSMA-(N)CIR at concentrations of 1 nM, 10 nM and 100 nM. The 3 components were co-cultured for 4 days with 9 images per well taken every 8 hours. Samples were run in triplicate. LNCaP tumor cells alone were also included as a control. The tumor cells were counted at each time point using the Incucyte S3's cell by cell analysis software. The values were normalized to the starting cell count for each well. The area under the tumor cell growth curves (AUC) was analyzed using PRISM Graphpad and used to calculate the % cytotoxicity. Percent cytotoxicity was calculated as:% Cytotoxicity=((AUC Tumor Alone-AUC Sample)/(AUC Tumor Alone))×100%.

Proliferation Analysis of Adapter Functionalized Engineered ABT cells: Anti-DNP TAC ABT cells, Anti-DAP12 SAR ABT cells, and Anti-CD28 CAR ABT cells (5E5 cells) labelled with CellTrace Violet dye were incubated with 1 nM, 10 nM, or 100 nM PSMA-CIR or PSMA-(N)CIR, and 293-PSMA or 293-WT tumor targets at an effector: target ratio 1:1 in a 24 well plate. Each engineered T cell was also cultured in media alone as a control to compare to. All proliferation assay samples were incubated for 3 days at 37° C. and 5% CO2. Cells were then stained with Live/Dead Fixable Near-IR stain, PerCP-Cy5.5-conjugated mouse anti-human CD8a, Alexa Fluor 700-conjugated mouse anti-human CD4, VioBright FITC-conjugated mouse anti-human NGFR, and BV605-conjugated mouse anti-human CD3. Flow cytometry data were acquired. Results were analysed with FCS Express by determining the starting generation peak based on the non-stimulated media alone sample and using the software proliferation package for fitting a proliferation model and collecting corresponding statistics, such as percent divided as displayed. CD4 and CD8 positive cells were analyzed separately and showed similar results.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### Sequences

Anti-DNP scFv (SPE7 VHVL) DNA sequence (SEQ ID NO: 1):
GAGGTGCAGCTCCAGCAGAGCGGCGCGGAGCTGGTTAAACCTGGCGCTTCCGTGAAGC
TGTCATGCAAAGCTTCGGGCTACACGTTCACCAGCTATTGGATGCATTGGGTCAAGCAG
AGGCCCGGCCGGGGGTTGGAGTGGATCGGTCGCATTGACCCCAACGGTGGCGGAACC
AAGTACAACGAGAAATTTAAGTCCAAGGCAACACTTACAGTCGATAAGCCTTCTTCCACG
GCCTACATGCAACTGAGCTCCCTGACCTCGGAGGACTCCGCAGTGTACTACTGTGCTCG
CATGTGGTACTACGGCACCTACTATTTCGACTATTGGGGCCAGGGGACTACTCTGACCG
TGTCCTCTGCCGCCGGGGGAGGCGGTTCTGGGGGTGGTGGATCTGGAGGGGGTGGCT
CCCAGGCCGTGGTGACCCAGGAGTCTGCGCTGACGACCAGCCCAGGGGAGACCGTCA
CCCTGACTTGCCGAAGTTCGACCGGCGCCGTCACAACCTCTAATTACGCCAACTGGGTG
CAGGAGAAGCCCGATCACCTGTTCACCGGCCTCATTGGAGGCACCAACAACCGCGCCC
CGGGCGTACCAGCGCGTTTTTCAGGCAGCCTCATCGGCAACAAGGCCGCACTGACCAT
CACCGGTGCTCAGACTGAAGACGAAGCCATCTACTTCTGTGCGCTTTGGTACTCCAATCA
CTTGGTGTTCGGCGGCGGCACGAAGCTCACTGTGCTGACC
Anti-DNP scFv (SPE7 VHVL) protein sequence (SEQ ID NO: 2):
EVQLQQSGAELVKPGASVKLSCKASGYTFTSYWMHWVKQRPGRGLEWIGRIDPNGGGTKY
NEKFKSKATLTVDKPSSTAYMQLSSLTSEDSAVYYCARMWYYGTYYFDYWGQGTTLTVSSA
AGGGGGGGGSGGGGSQAVVTQESALTTSPGETVTLTCRSSTGAVTTSNYANWVQEKPD
HLFTGLIGGTNNRAPGVPARFSGSLIGNKAALTITGAQTEDEAIYFCALWYSNHLVFGGGTKL
TVLT
Anti-DNP scFv (SPE7 VLVH) DNA sequence (SEQ ID NO: 3):
CAGGCCGTGGTGACCCAGGAGTCTGCGCTGACGACCAGCCCAGGGGAGACCGTCACC
CTGACTTGCCGAAGTTCGACCGGCGCCGTCACAACCTCTAATTACGCCAACTGGGTGCA
GGAGAAGCCCGATCACCTGTTCACCGGCCTCATTGGAGGCACCAACAACCGCGCCCCG
GGCGTACCAGCGCGTTTTTCAGGCAGCCTCATCGGCAACAAGGCCGCACTGACCATCAC
CGGTGCTCAGACTGAAGACGAAGCCATCTACTTCTGTGCGCTTTGGTACTCCAATCACTT
GGTGTTCGGCGGCGGCACGAAGCTCACTGTGCTGACCGGGGGAGGCGGTTCTGGGGG
TGGTGGATCTGGAGGGGGTGGCTCCGAGGTGCAGCTCCAGCAGAGCGGCGCGGAGCT
GGTTAAACCTGGCGCTTCCGTGAAGCTGTCATGCAAAGCTTCGGGCTACACGTTCACCA
GCTATTGGATGCATTGGGTCAAGCAGAGGCCCGGCCGGGGGTTGGAGTGGATCGGTCG
CATTGACCCCAACGGTGGCGGAACCAAGTACAACGAGAAATTTAAGTCCAAGGCAACAC
TTACAGTCGATAAGCCaTCTTCCACGGCCTACATGCAACTGAGCTCCCTGACCTCGGAG
GACTCCGCAGTGTACTACTGTGCTCGCATGTGGTACTACGGCACCTACTATTTCGACTAT
TGGGGCCAGGGGACTACTCTGACCGTGTCCTCTGCCGCC
Anti-DNP scFv (SPE7 VLVH) protein sequence (SEQ ID NO: 4):
QAVVTQESALTTSPGETVTLTCRSSTGAVTTSNYANWVQEKPDHLFTGLIGGTNNRAPGVPA
RFSGSLIGNKAALTITGAQTEDEAIYFCALWYSNHLVFGGGTKLTVLTGGGGSGGGGSGGG
GSEVQLQQSGAELVKPGASVKLSCKASGYTFTSYWMHWVKQRPGRGLEWIGRIDPNGGGT
KYNEKFKSKATLTVDKPSSTAYMQLSSLTSEDSAVYYCARMWYYGTYYFDYWGQGTTLTVS
SAA
TAC effector domain DNA sequence (SEQ ID NO: 5):
AACCCCGGGGGAGGAGGAGGGAGCGGGGGAGGAGGCAGCGGCGGGGGAGGCTCTGG
AGGAGGAGGGAGCGGATCCATGGATATCCAGATGACCCAGTCCCCGAGCTCCCTGTCC
GCCTCTGTGGGCGATAGGGTCACCATCACCTGCCGTGCCAGTCAGGACATCCGTAATTA
TCTGAACTGGTATCAACAGAAACCAGGAAAAGCTCCGAAACTACTGATTTACTATACCTC
CCGCCTGGAGTCTGGAGTCCCTTCTCGCTTCTCTGGTTCTGGTTCTGGGACGGATTACA
CTCTGACCATCAGCAGTCTGCAACCGGAAGACTTCGCAACTTATTACTGTCAGCAAGGTA
ATACTCTGCCGTGGACGTTCGGACAGGGCACCAAGGTGGAGATCAAAGGCGGCGGCGG
AAGTGGAGGAGGAGGCTCAGGCGGAGGAGGGAGCGAGGTTCAGCTGGTGGAGTCTGG
CGGTGGCCTGGTGCAGCCAGGGGGCTCACTCCGTTTGTCCTGTGCAGCTTCTGGCTACT
CCTTTACCGGCTACACTATGAACTGGGTGCGTCAGGCCCCAGGTAAGGGCCTGGAATGG
GTTGCACTGATTAATCCTTATAAAGGTGTTAGTACCTACAACCAGAAGTTCAAGGACCGTT
TCACTATAAGCGTAGATAAATCCAAAAACACAGCCTACCTGCAAATGAACAGCCTGCGTG
CTGAGGACACTGCCGTCTATTATTGTGCTAGAAGCGGATACTACGGCGATAGTGACTGG
TATTTTGACGTGTGGGGTCAAGGAACCCTGGTCACCGTCTCCTCGACTAGTGGCGGAGG
AGGATCACTCGAGAGCGGACAGGTGCTGCTGGAATCCAATATCAAAGTCCTGCCCACTT
GGTCTACCCCCGTGCAGCCTATGGCTCTGATTGTGCTGGGAGGAGTCGCAGGACTGCT
GCTGTTTATCGGGCTGGGAATTTTCTTTTGCGTGCGCTGCCGGCACCGGAGAAGGCAGG
CCGAGCGCATGAGCCAGATCAAGCGACTGCTGAGCGAGAAGAAAACCTGTCAGTGTCC
CCATAGATTCCAGAAGACCTGTTCACCCATTTGATAATCTAGATAGTAGATAGAATAGTAG
TAC effector domain protein sequence (SEQ ID NO: 6):
NPGGGGGSGGGGSGGGGSGGGGSGSMDIQMTQSPSSLSASVGDRVTITCRASQDIRNYL
NWYQQKPGKAPKLLIYYTSRLESGVPSRFSGSGSGTDYTLTISSLQPEDFATYYCQQGNTLP
WTFGQGTKVEIKGGGGSGGGGSGGGGSEVQLVESGGGLVQPGGSLRLSCAASGYSFTGY
TMNWVRQAPGKGLEWVALINPYKGVSTYNQKFKDRFTISVDKSKNTAYLQMNSLRAEDTAV
YYCARSGYYGDSDWYFDVWGQGTLVTVSSTSGGGGSLESGQVLLESNIKVLPTWSTPVQP
MALIVLGGVAGLLLFIGLGIFFCVRCRHRRRQAERMSQIKRLLSEKKTCQCPHRFQKTCSP|*
DAP12 DNA sequence (SEQ ID NO: 7):
ATGGGGGGACTTGAACCCTGCAGCAGGCTCCTGCTCCTGCCTCTCCTGCTGGCTGTAAG
TGGTCTCCGTCCTGTCCAGGCCCAGGCCCAGAGCGATTGCAGTTGCTCTACGGTGAGC
CCGGGCGTGCTGGCAGGGATCGTGATGGGAGACCTGGTGCTGACAGTGCTCATTGCCC
TGGCCGTGTACTTCCTGGGCCGGCTGGTCCCTCGGGGGCGAGGGGCTGCGGAGGCAG
CGACCCGGAAACAGCGTATCACTGAGACCGAGTCGCCTTATCAGGAGCTCCAGGGTCA
GAGGTCGGATGTCTACAGCGACCTCAACACACAGAGGCCGTATTACAAA
DAP12 protein sequence (SEQ ID NO: 8):
MGGLEPCSRLLLLPLLLAVSGLRPVQAQAQSDCSCSTVSPGVLAGIVMGDLVLTVLIALAVYF
LGRLVPRGRGAAEAATRKQRITETESPYQELQGQRSDVYSDLNTQRPYYK
KIR2SD2 Short Hinge effector domain DNA sequence (SEQ ID NO: 9):
AACGGATCCGGAGGAGGAGGGAGCGGAGGAGGAGGGAGCTCACCCACTGAACCAAGC
TCCAAAACCGGTAACCCCAGACACCTGCATGTTCTGATTGGGACCTCAGTGGTCAAAATC
CCTTTCACCATCCTCCTCTTCTTTCTCCTTCATCGCTGGTGCTCCAACAAAAAAAATGCTG
CTGTAATGGACCAAGAGCCTGCAGGGAACAGAACAGTGAACAGCGAGGATTCTGATGAA
CAAGACCATCAGGAGGTGTCATACGCATACGCATGATAACCTAGGTAGTAGATAGAATAG
TAG
KIR2SD2 Short Hinge effector domain protein sequence (SEQ ID NO: 10):
NGSGGGGSGGGGSSPTEPSSKTGNPRHLHVLIGTSVVKIPFTILLFFLLHRWCSNKKNAAVM
DQEPAGNRTVNSEDSDEQDHQEVSYAYA*
KIR2SD2 Long Hinge effector domain DNA sequence (SEQ ID NO: 11):
AACGGATCCGGAGGAGGAGGGAGCGGAGGAGGAGGGAGCGTCACAGGAAACCCTTCA
AATAGTTGGCCTTCACCCACTGAACCAAGCTCCAAAACCGGTAACCCCAGACACCTGCAT
GTTCTGATTGGGACCTCAGTGGTCAAAATCCCTTTCACCATCCTCCTCTTCTTTCTCCTTC
ATCGCTGGTGCTCCAACAAAAAAAATGCTGCTGTAATGGACCAAGAGCCTGCAGGGAAC
AGAACAGTGAACAGCGAGGATTCTGATGAACAAGACCATCAGGAGGTGTCATACGCATG
ATAACCTAGGTAGTAGATAGAATAGTAG
KIR2SD2 Long Hinge effector domain protein sequence (SEQ ID NO: 12):
NGSGGGGSGGGGSVTGNPSNSWPSPTEPSSKTGNPRHLHVLIGTSVVKIPFTILLFFLLHRW
CSNKKNAAVMDQEPAGNRTVNSEDSDEQDHQEVSYA*
CD28 CAR effector domain DNA sequence (SEQ ID NO: 13):
AACCGGATCCGTGGGGTCACCGTCTCTTCAGCGCTGAGCAACTCCATCATGTACTTCAG
CCACTTCGTGCCGGTCTTCCTGCCAGCGAAGCCCACCACGACGCCAGCGCCGCGACCA
CCAACACCGGCGCCCACCATCGCGTCGCAGCCCCTGTCCCTGCGCCCAGAGGCGTGCC
GGCCAGCGGCGGGGGGCGCAGTGCACACGAGGGGGCTGGACCCCTTTGGGTTTTGGG
TGCTGGTGGTGGTTGGTGGAGTCCTGGCTTGCTATAGCTTGCTAGTAACAGTGGCCTTT
ATTATTTTCTGGGTGAGGAGTAAGAGGAGCAGGCTCCTGCACAGTGACTACATGAACAT
GACTCCCCGCCGCCCCGGGCCCACCCGCAAGCATTACCAGCCCTATGCCCCACCACGC
GACTTCGCAGCCTATCGCTCCCTCGAGAGAGTGAGAGTGAAGTTCAGCAGGAGCGCAG
ACGCCCCCGCGTACCAGCAGGGCCAGAACCAGCTCTATAACGAGCTCAATCTAGGACGA
AGAGAGGAGTACGATGTTTTGGACAAGAGACGTGGCCGGGACCCTGAGATGGGGGGAA
AGCCGAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGCAC
GATGGCCTTTACCAGGGTCTCAGTACAGCCACCAAGGACACCTACGACGCCCTTCACAT
GCAGGCCCTGCCCCCTCGCTAATCTAGATAGTAGATAGAATAGTAG
CD28 CAR effector domain protein sequence (SEQ ID NO: 14):
NRIRGVTVSSALSNSIMYFSHFVPVFLPAKPTTTPAPRPPTPAPTIASQPLSLRPEACRPAAGG
AVHTRGLDPFGFWVLVVVGGVLACYSLLVTVAFIIFWVRSKRSRLLHSDYMNMTPRRPGPTR
KHYQPYAPPRDFAAYRSLERVRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATK
DTYDALHMQALPPR*
4-1BB CAR effector domain DNA sequence (SEQ ID NO: 15):
AACCGGATCCGTGGGGTCACCGTCTCCTCAGCGCTGAGCAACTCCATCATGTACTTCAG
CCACTTCGTGCCGGTCTTCCTGCCAGCGAAGCCCACCACGACGCCAGCGCCGCGACCA
CCAACACCGGCGCCCACCATCGCGTCGCAGCCCCTGTCCCTGCGCCCAGAGGCGTGCC
GGCCAGCGGCGGGGGGCGCAGTGCACACGAGGGGGCTGGACTTCGCCTGTGATATCT
ACATCTGGGCGCCCTTGGCCGGGACTTGTGGGGTCCTTCTCCTGTCACTGGTTATCACC
CTTTACTGCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAAGAA
GGAGGATGTGAACTGAGAGTGAAGTTCAGCAGGAGCGCAGACGCCCCCGCGTACAAGC
AGGGCCAGAACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCCCCTCGC
TAATCTAGATAGTAGATAGAATAGTAG
4-1BB CAR effector domain protein sequence (SEQ ID NO: 16):
NRIRGVTVSSALSNSIMYFSHFVPVFLPAKPTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEEDGC
SCRFPEEEEGGCELRVKFSRSADAPAYKQGQNQLYNELNLGRREEYDVLDKRRGRDPEMG
GKPRRKNPQEGLYNELQKDKMAEAYSEIGMKGERRRGKGHDGLYQGLSTATKDTYDALHM
QALPPR*
Anti-DNP TAC (SPE7 VHVL scFv) DNA sequence (SEQ ID NO: 17):
ATGGCTCTTCCTGTGACCGCGTTGCTACTGCCCCTGGCGCTGCTCCTGCACGCCGCTCG
CCCGGAGGTGCAGCTCCAGCAGAGCGGCGCGGAGCTGGTTAAACCTGGCGCTTCCGTG
AAGCTGTCATGCAAAGCTTCGGGCTACACGTTCACCAGCTATTGGATGCATTGGGTCAA
GCAGAGGCCCGGCCGGGGGTTGGAGTGGATCGGTCGCATTGACCCCAACGGTGGCGG
AACCAAGTACAACGAGAAATTTAAGTCCAAGGCAACACTTACAGTCGATAAGCCTTCTTC
CACGGCCTACATGCAACTGAGCTCCCTGACCTCGGAGGACTCCGCAGTGTACTACTGTG
CTCGCATGTGGTACTACGGCACCTACTATTTCGACTATTGGGGCCAGGGGACTACTCTG
ACCGTGTCCTCTGCCGCCGGGGGAGGCGGTTCTGGGGGTGGTGGATCTGGAGGGGGT
GGCTCCCAGGCCGTGGTGACCCAGGAGTCTGCGCTGACGACCAGCCCAGGGGAGACC
GTCACCCTGACTTGCCGAAGTTCGACCGGCGCCGTCACAACCTCTAATTACGCCAACTG
GGTGCAGGAGAAGCCCGATCACCTGTTCACCGGCCTCATTGGAGGCACCAACAACCGC
GCCCCGGGCGTACCAGCGCGTTTTTCAGGCAGCCTCATCGGCAACAAGGCCGCACTGA
CCATCACCGGTGCTCAGACTGAAGACGAAGCCATCTACTTCTGTGCGCTTTGGTACTCCA
ATCACTTGGTGTTCGGCGGCGGCACGAAGCTCACTGTGCTGACCCTGCAGAAACTGAAT
GAACAGAAACTGATTAGCGAAGAAGACCTGAACCCCGGGGGAGGAGGAGGGAGCGGG
GGAGGAGGCAGCGGCGGGGGAGGCTCTGGAGGAGGAGGGAGCGGATCCATGGATATC
CAGATGACCCAGTCCCCGAGCTCCCTGTCCGCCTCTGTGGGCGATAGGGTCACCATCAC
CTGCCGTGCCAGTCAGGACATCCGTAATTATCTGAACTGGTATCAACAGAAACCAGGAAA
AGCTCCGAAACTACTGATTTACTATACCTCCCGCCTGGAGTCTGGAGTCCCTTCTCGCTT
CTCTGGTTCTGGTTCTGGGACGGATTACACTCTGACCATCAGCAGTCTGCAACCGGAAG
ACTTCGCAACTTATTACTGTCAGCAAGGTAATACTCTGCCGTGGACGTTCGGACAGGGCA
CCAAGGTGGAGATCAAAGGCGGCGGCGGAAGTGGAGGAGGAGGCTCAGGCGGAGGAG
GGAGCGAGGTTCAGCTGGTGGAGTCTGGCGGTGGCCTGGTGCAGCCAGGGGGCTCAC
TCCGTTTGTCCTGTGCAGCTTCTGGCTACTCCTTTACCGGCTACACTATGAACTGGGTGC
GTCAGGCCCCAGGTAAGGGCCTGGAATGGGTTGCACTGATTAATCCTTATAAAGGTGTT
AGTACCTACAACCAGAAGTTCAAGGACCGTTTCACTATAAGCGTAGATAAATCCAAAAAC
ACAGCCTACCTGCAAATGAACAGCCTGCGTGCTGAGGACACTGCCGTCTATTATTGTGCT
AGAAGCGGATACTACGGCGATAGTGACTGGTATTTTGACGTGTGGGGTCAAGGAACCCT
GGTCACCGTCTCCTCGACTAGTGGCGGAGGAGGATCACTCGAGAGCGGACAGGTGCTG
CTGGAATCCAATATCAAAGTCCTGCCCACTTGGTCTACCCCCGTGCAGCCTATGGCTCTG
ATTGTGCTGGGAGGAGTCGCAGGACTGCTGCTGTTTATCGGGCTGGGAATTTTCTTTTG
CGTGCGCTGCCGGCACCGGAGAAGGCAGGCCGAGCGCATGAGCCAGATCAAGCGACT
GCTGAGCGAGAAGAAAACCTGTCAGTGTCCCCATAGATTCCAGAAGACCTGTTCACCCA
TTTGATAATCTAGATAGTAGATAGAATAGTAG
Anti-DNP TAC (SPE7 VHVL scFv) protein sequence (SEQ ID NO: 18):
MALPVTALLLPLALLLHAARPEVQLQQSGAELVKPGASVKLSCKASGYTFTSYWMHWVKQR
PGRGLEWIGRIDPNGGGTKYNEKFKSKATLTVDKPSSTAYMQLSSLTSEDSAVYYCARMWY
YGTYYFDYWGQGTTLTVSSAAGGGGSGGGGSGGGGSQAVVTQESALTTSPGETVTLTCRS
STGAVTTSNYANWVQEKPDHLFTGLIGGTNNRAPGVPARFSGSLIGNKAALTITGAQTEDEAI
YFCALWYSNHLVFGGGTKLTVLTLQKLNEQKLISEEDLNPGGGGGSGGGGSGGGGSGGGG
SGSMDIQMTQSPSSLSASVGDRVTITCRASQDIRNYLNWYQQKPGKAPKLLIYYTSRLESGV
PSRFSGSGSGTDYTLTISSLQPEDFATYYCQQGNTLPWTFGQGTKVEIKGGGGSGGGGSG
GGGSEVQLVESGGGLVQPGGSLRLSCAASGYSFTGYTMNWVRQAPGKGLEWVALINPYKG
VSTYNQKFKDRFTISVDKSKNTAYLQMNSLRAEDTAVYYCARSGYYGDSDWYFDVWGQGT
LVTVSSTSGGGGSLESGQVLLESNIKVLPTWSTPVQPMALIVLGGVAGLLLFIGLGIFFCVRCR
HRRRQAERMSQIKRLLSEKKTCQCPHRFQKTCSPI*
Anti-DNP TAC (SPE7 VLVH scFv) DNA sequence (SEQ ID NO: 19):
ATGGCTCTTCCTGTGACCGCGTTGCTACTGCCCCTGGCGCTGCTCCTGCACGCCGCTCG
CCCGCAGGCCGTGGTGACCCAGGAGTCTGCGCTGACGACCAGCCCAGGGGAGACCGT
CACCCTGACTTGCCGAAGTTCGACCGGCGCCGTCACAACCTCTAATTACGCCAACTGGG
TGCAGGAGAAGCCCGATCACCTGTTCACCGGCCTCATTGGAGGCACCAACAACCGCGC
CCCGGGCGTACCAGCGCGTTTTTCAGGCAGCCTCATCGGCAACAAGGCCGCACTGACC
ATCACCGGTGCTCAGACTGAAGACGAAGCCATCTACTTCTGTGCGCTTTGGTACTCCAAT
CACTTGGTGTTCGGCGGCGGCACGAAGCTCACTGTGCTGACCGGGGGAGGCGGTTCTG
GGGGTGGTGGATCTGGAGGGGGTGGCTCCGAGGTGCAGCTCCAGCAGAGCGGCGCGG
AGCTGGTTAAACCTGGCGCTTCCGTGAAGCTGTCATGCAAAGCTTCGGGCTACACGTTC
ACCAGCTATTGGATGCATTGGGTCAAGCAGAGGCCCGGCCGGGGGTTGGAGTGGATCG
GTCGCATTGACCCCAACGGTGGCGGAACCAAGTACAACGAGAAATTTAAGTCCAAGGCA
ACACTTACAGTCGATAAGCCATCTTCCACGGCCTACATGCAACTGAGCTCCCTGACCTCG
GAGGACTCCGCAGTGTACTACTGTGCTCGCATGTGGTACTACGGCACCTACTATTTCGA
CTATTGGGGCCAGGGGACTACTCTGACCGTGTCCTCTGCCGCCCTGCAGAAACTGAATG
AACAGAAACTGATTAGCGAAGAAGACCTGAACCCCGGGGGAGGAGGAGGGAGCGGGG
GAGGAGGCAGCGGCGGGGGAGGCTCTGGAGGAGGAGGGAGCGGATCCATGGATATCC
AGATGACCCAGTCCCCGAGCTCCCTGTCCGCCTCTGTGGGCGATAGGGTCACCATCACC
TGCCGTGCCAGTCAGGACATCCGTAATTATCTGAACTGGTATCAACAGAAACCAGGAAAA
GCTCCGAAACTACTGATTTACTATACCTCCCGCCTGGAGTCTGGAGTCCCTTCTCGCTTC
TCTGGTTCTGGTTCTGGGACGGATTACACTCTGACCATCAGCAGTCTGCAACCGGAAGA
CTTCGCAACTTATTACTGTCAGCAAGGTAATACTCTGCCGTGGACGTTCGGACAGGGCA
CCAAGGTGGAGATCAAAGGCGGCGGCGGAAGTGGAGGAGGAGGCTCAGGCGGAGGAG
GGAGCGAGGTTCAGCTGGTGGAGTCTGGCGGTGGCCTGGTGCAGCCAGGGGGCTCAC
TCCGTTTGTCCTGTGCAGCTTCTGGCTACTCCTTTACCGGCTACACTATGAACTGGGTGC
GTCAGGCCCCAGGTAAGGGCCTGGAATGGGTTGCACTGATTAATCCTTATAAAGGTGTT
AGTACCTACAACCAGAAGTTCAAGGACCGTTTCACTATAAGCGTAGATAAATCCAAAAAC
ACAGCCTACCTGCAAATGAACAGCCTGCGTGCTGAGGACACTGCCGTCTATTATTGTGCT
AGAAGCGGATACTACGGCGATAGTGACTGGTATTTTGACGTGTGGGGTCAAGGAACCCT
GGTCACCGTCTCCTCGACTAGTGGCGGAGGAGGATCACTCGAGAGCGGACAGGTGCTG
CTGGAATCCAATATCAAAGTCCTGCCCACTTGGTCTACCCCCGTGCAGCCTATGGCTCTG
ATTGTGCTGGGAGGAGTCGCAGGACTGCTGCTGTTTATCGGGCTGGGAATTTTCTTTTG
CGTGCGCTGCCGGCACCGGAGAAGGCAGGCCGAGCGCATGAGCCAGATCAAGCGACT
GCTGAGCGAGAAGAAAACCTGTCAGTGTCCCCATAGATTCCAGAAGACCTGTTCACCCA
TTTGATAATCTAGATAGTAGATAGAATAGTAG
Anti-DNP TAC (SPE7 VLVH scFv) protein sequence (SEQ ID NO: 20):
MALPVTALLLPLALLLHAARPQAVVTQESALTTSPGETVTLTCRSSTGAVTTSNYANWVQEKP
DHLFTGLIGGTNNRAPGVPARFSGSLIGNKAALTITGAQTEDEAIYFCALWYSNHLVFGGGTK
LTVLTGGGGSGGGGSGGGGSEVQLQQSGAELVKPGASVKLSCKASGYTFTSYWMHWVKQ
RPGRGLEWIGRIDPNGGGTKYNEKFKSKATLTVDKPSSTAYMQLSSLTSEDSAVYYCARMW
YYGTYYFDYWGQGTTLTVSSAALQKLNEQKLISEEDLNPGGGGGSGGGGSGGGGSGGGG
SGSMDIQMTQSPSSLSASVGDRVTITCRASQDIRNYLNWYQQKPGKAPKLLIYYTSRLESGV
PSRFSGSGSGTDYTLTISSLQPEDFATYYCQQGNTLPWTFGQGTKVEIKGGGGGGGGSG
GGGSEVQLVESGGGLVQPGGSLRLSCAASGYSFTGYTMNWVRQAPGKGLEWVALINPYKG
VSTYNQKFKDRFTISVDKSKNTAYLQMNSLRAEDTAVYYCARSGYYGDSDWYFDVWGQGT
LVTVSSTSGGGGSLESGQVLLESNIKVLPTWSTPVQPMALIVLGGVAGLLLFIGLGIFFCVRCR
HRRRQAERMSQIKRLLSEKKTCQCPHRFQKTCSPI*
Dap12 + Anti-DNP (SPE7 VHVL) KIR2DS2 Short Hinge SAR DNA sequence (SEQ
ID NO: 21):
ATGGGGGGACTTGAACCCTGCAGCAGGCTCCTGCTCCTGCCTCTCCTGCTGGCTGTAAG
TGGTCTCCGTCCTGTCCAGGCCCAGGCCCAGAGCGATTGCAGTTGCTCTACGGTGAGC
CCGGGCGTGCTGGCAGGGATCGTGATGGGAGACCTGGTGCTGACAGTGCTCATTGCCC
TGGCCGTGTACTTCCTGGGCCGGCTGGTCCCTCGGGGGCGAGGGGCTGCGGAGGCAG
CGACCCGGAAACAGCGTATCACTGAGACCGAGTCGCCTTATCAGGAGCTCCAGGGTCA
GAGGTCGGATGTCTACAGCGACCTCAACACACAGAGGCCGTATTACAAAGGCAGCGGC
GAGGGCAGAGGCTCCCTGCTGACCTGCGGGGACGTGGAAGAGAATCCTGGCCCTGGCA
CCAGCCGTACGATGGCTCTTCCTGTGACCGCGTTGCTACTGCCCCTGGCGCTGCTCCTG
CACGCCGCTCGCCCGGAGGTGCAGCTCCAGCAGAGCGGCGCGGAGCTGGTTAAACCT
GGCGCTTCCGTGAAGCTGTCATGCAAAGCTTCGGGCTACACGTTCACCAGCTATTGGAT
GCATTGGGTCAAGCAGAGGCCCGGCCGGGGGTTGGAGTGGATCGGTCGCATTGACCCC
AACGGTGGCGGAACCAAGTACAACGAGAAATTTAAGTCCAAGGCAACACTTACAGTCGA
TAAGCCTTCTTCCACGGCCTACATGCAACTGAGCTCCCTGACCTCGGAGGACTCCGCAG
TGTACTACTGTGCTCGCATGTGGTACTACGGCACCTACTATTTCGACTATTGGGGCCAGG
GGACTACTCTGACCGTGTCCTCTGCCGCCGGGGGAGGCGGTTCTGGGGGTGGTGGATC
TGGAGGGGGTGGCTCCCAGGCCGTGGTGACCCAGGAGTCTGCGCTGACGACCAGCCC
AGGGGAGACCGTCACCCTGACTTGCCGAAGTTCGACCGGCGCCGTCACAACCTCTAATT
ACGCCAACTGGGTGCAGGAGAAGCCCGATCACCTGTTCACCGGCCTCATTGGAGGCAC
CAACAACCGCGCCCCGGGCGTACCAGCGCGTTTTTCAGGCAGCCTCATCGGCAACAAG
GCCGCACTGACCATCACCGGTGCTCAGACTGAAGACGAAGCCATCTACTTCTGTGCGCT
TTGGTACTCCAATCACTTGGTGTTCGGCGGCGGCACGAAGCTCACTGTGCTGACCCTGC
AGAAACTGAATGAACAGAAACTGATTAGCGAAGAAGACCTGAACGGATCCGGAGGAGGA
GGGAGCGGAGGAGGAGGGAGCTCACCCACTGAACCAAGCTCCAAAACCGGTAACCCCA
GACACCTGCATGTTCTGATTGGGACCTCAGTGGTCAAAATCCCTTTCACCATCCTCCTCT
TCTTTCTCCTTCATCGCTGGTGCTCCAACAAAAAAAATGCTGCTGTAATGGACCAAGAGC
CTGCAGGGAACAGAACAGTGAACAGCGAGGATTCTGATGAACAAGACCATCAGGAGGTG
TCATACGCATACGCATGATAACCTAGGTAGTAGATAGAATAGTAG
Dap12 + Anti-DNP (SPE7 VHVL) KIR2DS2 Short Hinge SAR protein sequence
(SEQ ID NO: 22):
MGGLEPCSRLLLLPLLLAVSGLRPVQAQAQSDCSCSTVSPGVLAGIVMGDLVLTVLIALAVYF
LGRLVPRGRGAAEAATRKQRITETESPYQELQGQRSDVYSDLNTQRPYYKGSGEGRGSLLT
CGDVEENPGPGTSRTMALPVTALLLPLALLLHAARPEVQLQQSGAELVKPGASVKLSCKASG
YTFTSYWMHWVKQRPGRGLEWIGRIDPNGGGTKYNEKFKSKATLTVDKPSSTAYMQLSSLT
SEDSAVYYCARMWYYGTYYFDYWGQGTTLTVSSAAGGGGSGGGGSGGGGSQAVVTQES
ALTTSPGETVTLTCRSSTGAVTTSNYANWVQEKPDHLFTGLIGGTNNRAPGVPARFSGSLIG
NKAALTITGAQTEDEAIYFCALWYSNHLVFGGGTKLTVLTLQKLNEQKLISEEDLNGSGGGGS
GGGGSSPTEPSSKTGNPRHLHVLIGTSVVKIPFTILLFFLLHRWCSNKKNAAVMDQEPAGNR
TVNSEDSDEQDHQEVSYAYA*
Dap12 + Anti-DNP (SPE7 VHVL) KIR2DS2 Long Hinge SAR DNA sequence (SEQ
ID NO: 23):
ATGGGGGGACTTGAACCCTGCAGCAGGCTCCTGCTCCTGCCTCTCCTGCTGGCTGTAAG
TGGTCTCCGTCCTGTCCAGGCCCAGGCCCAGAGCGATTGCAGTTGCTCTACGGTGAGC
CCGGGCGTGCTGGCAGGGATCGTGATGGGAGACCTGGTGCTGACAGTGCTCATTGCCC
TGGCCGTGTACTTCCTGGGCCGGCTGGTCCCTCGGGGGCGAGGGGCTGCGGAGGCAG
CGACCCGGAAACAGCGTATCACTGAGACCGAGTCGCCTTATCAGGAGCTCCAGGGTCA
GAGGTCGGATGTCTACAGCGACCTCAACACACAGAGGCCGTATTACAAAGGCAGCGGC
GAGGGCAGAGGCTCCCTGCTGACCTGCGGGGACGTGGAAGAGAATCCTGGCCCTGGCA
CCAGCCGTACGATGGCTCTTCCTGTGACCGCGTTGCTACTGCCCCTGGCGCTGCTCCTG
CACGCCGCTCGCCCGGAGGTGCAGCTCCAGCAGAGCGGCGCGGAGCTGGTTAAACCT
GGCGCTTCCGTGAAGCTGTCATGCAAAGCTTCGGGCTACACGTTCACCAGCTATTGGAT
GCATTGGGTCAAGCAGAGGCCCGGCCGGGGGTTGGAGTGGATCGGTCGCATTGACCCC
AACGGTGGCGGAACCAAGTACAACGAGAAATTTAAGTCCAAGGCAACACTTACAGTCGA
TAAGCCTTCTTCCACGGCCTACATGCAACTGAGCTCCCTGACCTCGGAGGACTCCGCAG
TGTACTACTGTGCTCGCATGTGGTACTACGGCACCTACTATTTCGACTATTGGGGCCAGG
GGACTACTCTGACCGTGTCCTCTGCCGCCGGGGGAGGCGGTTCTGGGGGTGGTGGATC
TGGAGGGGGTGGCTCCCAGGCCGTGGTGACCCAGGAGTCTGCGCTGACGACCAGCCC
AGGGGAGACCGTCACCCTGACTTGCCGAAGTTCGACCGGCGCCGTCACAACCTCTAATT
ACGCCAACTGGGTGCAGGAGAAGCCCGATCACCTGTTCACCGGCCTCATTGGAGGCAC
CAACAACCGCGCCCCGGGCGTACCAGCGCGTTTTTCAGGCAGCCTCATCGGCAACAAG
GCCGCACTGACCATCACCGGTGCTCAGACTGAAGACGAAGCCATCTACTTCTGTGCGCT
TTGGTACTCCAATCACTTGGTGTTCGGCGGCGGCACGAAGCTCACTGTGCTGACCCTGC
AGAAACTGAATGAACAGAAACTGATTAGCGAAGAAGACCTGAACGGATCCGGAGGAGGA
GGGAGCGGAGGAGGAGGGAGCGTCACAGGAAACCCTTCAAATAGTTGGCCTTCACCCA
CTGAACCAAGCTCCAAAACCGGTAACCCCAGACACCTGCATGTTCTGATTGGGACCTCA
GTGGTCAAAATCCCTTTCACCATCCTCCTCTTCTTTCTCCTTCATCGCTGGTGCTCCAACA
AAAAAAATGCTGCTGTAATGGACCAAGAGCCTGCAGGGAACAGAACAGTGAACAGCGAG
GATTCTGATGAACAAGACCATCAGGAGGTGTCATACGCATGATAACCTAGGTAGTAGATA
GAATAGTAG
Dap12 + Anti-DNP (SPE7 VHVL) KIR2DS2 Long Hinge SAR protein sequence
(SEQ ID NO: 24):
MGGLEPCSRLLLLPLLLAVSGLRPVQAQAQSDCSCSTVSPGVLAGIVMGDLVLTVLIALAVYF
LGRLVPRGRGAAEAATRKQRITETESPYQELQGQRSDVYSDLNTQRPYYKGSGEGRGSLLT
CGDVEENPGPGTSRTMALPVTALLLPLALLLHAARPEVQLQQSGAELVKPGASVKLSCKASG
YTFTSYWMHWVKQRPGRGLEWIGRIDPNGGGTKYNEKFKSKATLTVDKPSSTAYMQLSSLT
SEDSAVYYCARMWYYGTYYFDYWGQGTTLTVSSAAGGGGSGGGGSGGGGSQAVVTQES
ALTTSPGETVTLTCRSSTGAVTTSNYANWVQEKPDHLFTGLIGGTNNRAPGVPARFSGSLIG
NKAALTITGAQTEDEAIYFCALWYSNHLVFGGGTKLTVLTLQKLNEQKLISEEDLINGSGGGGS
GGGGSVTGNPSNSWPSPTEPSSKTGNPRHLHVLIGTSVVKIPFTILLFFLLHRWCSNKKNAAV
MDQEPAGNRTVNSEDSDEQDHQEVSYA*
Anti-DNP (SPE7 VHVL) CD28 CAR DNA sequence (SEQ ID NO: 25):
ATGGCTCTTCCTGTGACCGCGTTGCTACTGCCCCTGGCGCTGCTCCTGCACGCCGCTCG
CCCGGAGGTGCAGCTCCAGCAGAGCGGCGCGGAGCTGGTTAAACCTGGCGCTTCCGTG
AAGCTGTCATGCAAAGCTTCGGGCTACACGTTCACCAGCTATTGGATGCATTGGGTCAA
GCAGAGGCCCGGCCGGGGGTTGGAGTGGATCGGTCGCATTGACCCCAACGGTGGCGG
AACCAAGTACAACGAGAAATTTAAGTCCAAGGCAACACTTACAGTCGATAAGCCTTCTTC
CACGGCCTACATGCAACTGAGCTCCCTGACCTCGGAGGACTCCGCAGTGTACTACTGTG
CTCGCATGTGGTACTACGGCACCTACTATTTCGACTATTGGGGCCAGGGGACTACTCTG
ACCGTGTCCTCTGCCGCCGGGGGAGGCGGTTCTGGGGGTGGTGGATCTGGAGGGGGT
GGCTCCCAGGCCGTGGTGACCCAGGAGTCTGCGCTGACGACCAGCCCAGGGGAGACC
GTCACCCTGACTTGCCGAAGTTCGACCGGCGCCGTCACAACCTCTAATTACGCCAACTG
GGTGCAGGAGAAGCCCGATCACCTGTTCACCGGCCTCATTGGAGGCACCAACAACCGC
GCCCCGGGCGTACCAGCGCGTTTTTCAGGCAGCCTCATCGGCAACAAGGCCGCACTGA
CCATCACCGGTGCTCAGACTGAAGACGAAGCCATCTACTTCTGTGCGCTTTGGTACTCCA
ATCACTTGGTGTTCGGCGGCGGCACGAAGCTCACTGTGCTGACCCTGCAGAAACTGAAT
GAACAGAAACTGATTAGCGAAGAAGACCTGAACCGGATCCGTGGGGTCACCGTCTCTTC
AGCGCTGAGCAACTCCATCATGTACTTCAGCCACTTCGTGCCGGTCTTCCTGCCAGCGA
AGCCCACCACGACGCCAGCGCCGCGACCACCAACACCGGCGCCCACCATCGCGTCGCA
GCCCCTGTCCCTGCGCCCAGAGGCGTGCCGGCCAGCGGCGGGGGGCGCAGTGCACAC
GAGGGGGCTGGACCCCTTTGGGTTTTGGGTGCTGGTGGTGGTTGGTGGAGTCCTGGCT
TGCTATAGCTTGCTAGTAACAGTGGCCTTTATTATTTTCTGGGTGAGGAGTAAGAGGAGC
AGGCTCCTGCACAGTGACTACATGAACATGACTCCCCGCCGCCCCGGGCCCACCCGCA
AGCATTACCAGCCCTATGCCCCACCACGCGACTTCGCAGCCTATCGCTCCCTCGAGAGA
GTGAGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAGAGA
CGTGGCCGGGACCCTGAGATGGGGGGAAAGCCGAGAAGGAAGAACCCTCAGGAAGGC
CTGTACAATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTACAGC
CACCAAGGACACCTACGACGCCCTTCACATGCAGGCCCTGCCCCCTCGCTAATCTAGAT
AGTAGATAGAATAGTAG
Anti-DNP (SPE7 VHVL) CD28 CAR protein sequence (SEQ ID NO: 26):
MALPVTALLLPLALLLHAARPEVQLQQSGAELVKPGASVKLSCKASGYTFTSYWMHWVKQR
PGRGLEWIGRIDPNGGGTKYNEKFKSKATLTVDKPSSTAYMQLSSLTSEDSAVYYCARMWY
YGTYYFDYWGQGTTLTVSSAAGGGGGGGGSGGGGSQAVVTQESALTTSPGETVTLTCRS
STGAVTTSNYANWVQEKPDHLFTGLIGGTNNRAPGVPARFSGSLIGNKAALTITGAQTEDEAI
YFCALWYSNHLVFGGGTKLTVLTLQKLNEQKLISEEDLNRIRGVTVSSALSNSIMYFSHFVPVF
LPAKPTTTPAPRPPTPAPTIASQPLSLRPEACRPAAGGAVHTRGLDPFGFWVLVVVGGVLAC
YSLLVTVAFIIFWVRSKRSRLLHSDYMNMTPRRPGPTRKHYQPYAPPRDFAAYRSLERVRVK
FSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR*
Anti-DNP (SPE7 VHVL) 4-1BB CAR DNA sequence (SEQ ID NO: 27):
ATGGCTCTTCCTGTGACCGCGTTGCTACTGCCCCTGGCGCTGCTCCTGCACGCCGCTCG
CCCGGAGGTGCAGCTCCAGCAGAGCGGCGCGGAGCTGGTTAAACCTGGCGCTTCCGTG
AAGCTGTCATGCAAAGCTTCGGGCTACACGTTCACCAGCTATTGGATGCATTGGGTCAA
GCAGAGGCCCGGCCGGGGGTTGGAGTGGATCGGTCGCATTGACCCCAACGGTGGCGG
AACCAAGTACAACGAGAAATTTAAGTCCAAGGCAACACTTACAGTCGATAAGCCTTCTTC
CACGGCCTACATGCAACTGAGCTCCCTGACCTCGGAGGACTCCGCAGTGTACTACTGTG
CTCGCATGTGGTACTACGGCACCTACTATTTCGACTATTGGGGCCAGGGGACTACTCTG
ACCGTGTCCTCTGCCGCCGGGGGAGGCGGTTCTGGGGGTGGTGGATCTGGAGGGGGT
GGCTCCCAGGCCGTGGTGACCCAGGAGTCTGCGCTGACGACCAGCCCAGGGGAGACC
GTCACCCTGACTTGCCGAAGTTCGACCGGCGCCGTCACAACCTCTAATTACGCCAACTG
GGTGCAGGAGAAGCCCGATCACCTGTTCACCGGCCTCATTGGAGGCACCAACAACCGC
GCCCCGGGCGTACCAGCGCGTTTTTCAGGCAGCCTCATCGGCAACAAGGCCGCACTGA
CCATCACCGGTGCTCAGACTGAAGACGAAGCCATCTACTTCTGTGCGCTTTGGTACTCCA
ATCACTTGGTGTTCGGCGGCGGCACGAAGCTCACTGTGCTGACCCTGCAGAAACTGAAT
GAACAGAAACTGATTAGCGAAGAAGACCTGAACCGGATCCGTGGGGTCACCGTCTCCTC
AGCGCTGAGCAACTCCATCATGTACTTCAGCCACTTCGTGCCGGTCTTCCTGCCAGCGA
AGCCCACCACGACGCCAGCGCCGCGACCACCAACACCGGCGCCCACCATCGCGTCGCA
GCCCCTGTCCCTGCGCCCAGAGGCGTGCCGGCCAGCGGCGGGGGGCGCAGTGCACAC
GAGGGGGCTGGACTTCGCCTGTGATATCTACATCTGGGCGCCCTTGGCCGGGACTTGT
GGGGTCCTTCTCCTGTCACTGGTTATCACCCTTTACTGCAAACGGGGCAGAAAGAAACTC
CTGTATATATTCAAACAACCATTTATGAGACCAGTACAAACTACTCAAGAGGAAGATGGCT
GTAGCTGCCGATTTCCAGAAGAAGAAGAAGGAGGATGTGAACTGAGAGTGAAGTTCAGC
AGGAGCGCAGACGCCCCCGCGTACAAGCAGGGCCAGAACCAGCTCTATAACGAGCTCA
ATCTAGGACGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATGAACTGCAG
AAAGATAAGATGGCGGAGGCCTACAGTGAGATTGGGATGAAAGGCGAGCGCCGGAGGG
GCAAGGGGCACGATGGCCTTTACCAGGGTCTCAGTACAGCCACCAAGGACACCTACGA
CGCCCTTCACATGCAGGCCCTGCCCCCTCGCTAATCTAGATAGTAGATAGAATAGTAG
Anti-DNP (SPE7 VHVL) 4-1BB CAR protein sequence (SEQ ID NO: 28):
MGAGATGRAMDGPRLLLLLLLGVSLGGAKEACPTGLYTHSGECCKACNLGEGVAQPCGAN
QTVCEPCLDSVTFSDVVSATEPCKPCTECVGLQSMSAPCVEADDAVCRCAYGYYQDETTG
RCEACRVCEAGSGLVFSCQDKQNTVCEECPDGTYSDEANHVDPCLPCTVCEDTERQLREC
TRWADAECEEIPGRWITRSTPPEGSDSTAPSTQEPEAPPEQDLIASTVAGVVTTVMGSSQPV
VTRGTTDNLIPVYCSILAAVVVGLVAYIAFKRWNRGIFTSGSGEGRGSLLTCGDVEENPGPGT
SGTMALPVTALLLPLALLLHAARPEVQLQQSGAELVKPGASVKLSCKASGYTFTSYWMHWV
KQRPGRGLEWIGRIDPNGGGTKYNEKFKSKATLTVDKPSSTAYMQLSSLTSEDSAVYYCAR
MWYYGTYYFDYWGQGTTLTVSSAAGGGGSGGGGSGGGGSQAVVTQESALTTSPGETVTL
TCRSSTGAVTTSNYANWVQEKPDHLFTGLIGGTNNRAPGVPARFSGSLIGNKAALTITGAQT
EDEAIYFCALWYSNHLVFGGGTKLTVLTLQKLNEQKLISEEDLNRIRGVTVSSALSNSIMYFSH
FVPVFLPAKPTTTPAPRPPTPAPTIASQPLSLRPEACRPAAGGAVHTRGLDFACDIYIWAPLA
GTCGVLLLSLVITLYCKRGRKKLLYIFKQPFMRPVQTTQEEDGCSCRFPEEEEGGCELRVKFS
RSADAPAYKQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR*
Synthetic uPAR peptide ligand
(SEQ ID NO: 29)
L-Lys-Gly-Gly-L-Ser-Gly-L-Asp-L-Cha-L-Phe-D-Ser-D-Arg-L-Tyr-L-Leu-L-Trp-L-Ser

### REFERENCES

1. Stevanovic S, Draper L M, Langhan M M, et al. Complete regression of metastatic cervical cancer after treatment with human papillomavirus-targeted tumor-infiltrating T cells. J Clin Oncol. May 10 2015; 33(14):1543-50. doi:10.1200/JCO.2014.58.9093
2. Sim G C, Chacon J, Haymaker C, et al. Tumor-infiltrating lymphocyte therapy for melanoma: rationale and issues for further clinical development. BioDrugs: clinical immunotherapeutics, biopharmaceuticals and gene therapy. October 2014; 28(5):421-37. doi:10.1007/s40259-014-0097-y
3. Maude S L, Frey N, Shaw P A, et al. Chimeric antigen receptor T cells for sustained remissions in leukemia. The New England journal of medicine. Oct. 16 2014; 371(16):1507-17. doi:10.1056/NEJMoa1407222
4. Heslop H E, Slobod K S, Pule M A, et al. Long-term outcome of EBV-specific T-cell infusions to prevent or treat EBV-related lymphoproliferative disease in transplant recipients. Blood. Feb. 4 2010; 115(5):925-35.
5. D'Angelo S P, Melchiori L, Merchant M S, et al. Antitumor Activity Associated with Prolonged Persistence of Adoptively Transferred N Y-ESO-1 (c259)T Cells in Synovial Sarcoma. Cancer Discov. August 2018; 8(8):944-957. doi:10.1158/2159-8290.CD-17-1417
6. Iwahori K, Kakarla S, Velasquez M P, et al. Engager T cells: a new class of antigen-specific T cells that redirect bystander T cells. Mol Ther. January 2015; 23(1):171-8. doi:10.1038/mt.2014.156
7. Geyer M B, Brentjens R J. Review: Current clinical applications of chimeric antigen receptor (CAR) modified T cells. Cytotherapy. November 2016; 18(11):1393-1409. doi:10.1016/j.jcyt.2016.07.003
8. Baeuerle P A, Ding J, Patel E, et al. Synthetic TRuC receptors engaging the complete T cell receptor for potent anti-tumor response. Nat Commun. May 7 2019; 10(1):2087. doi:10.1038/s41467-019-10097-0
9. Helsen C W, Hammill J A, Lau V W C, et al. The chimeric TAC receptor co-opts the T cell receptor yielding robust anti-tumor activity without toxicity. Nat Commun. Aug. 3 2018; 9(1):3049. doi:10.1038/s41467-018-05395-y
10. Xu Y, Yang Z, Horan L H, et al. A novel antibody-TCR (AbTCR) platform combines Fab-based antigen recognition with gamma/delta-TCR signaling to facilitate T-cell cytotoxicity with low cytokine release. Cell Discov. 2018; 4:62. doi:10.1038/s41421-018-0066-6
11. Larson R C, Maus M V. Recent advances and discoveries in the mechanisms and functions of CAR T cells. Nature reviews. March 2021; 21(3):145-161. doi:10.1038/s41568-020-00323-z
12. Mardiana S, Gill S. CAR T Cells for Acute Myeloid Leukemia: State of the Art and Future Directions. Front Oncol. 2020; 10:697. doi:10.3389/fonc.2020.00697
13. Mohyuddin G R, Rooney A, Balmaceda N, et al. Chimeric antigen receptor T-cell therapy in multiple myeloma: a systematic review and meta-analysis of 950 patients. Blood Adv. Feb. 23 2021; 5(4):1097-1101. doi:10.1182/bloodadvances.2020004017
14. Aamir S, Anwar M Y, Khalid F, Khan S I, Ali M A, Khattak Z E. Systematic Review and Meta-analysis of CD19-Specific CAR-T Cell Therapy in Relapsed/Refractory Acute Lymphoblastic Leukemia in the Pediatric and Young Adult Population: Safety and Efficacy Outcomes. Clin Lymphoma Myeloma Leuk. Dec. 17 2020; doi:10.1016/j.clml.2020.12.010
15. Reagan P M, Friedberg J W. Axicabtagene ciloleucel and brexucabtagene autoleucel in relapsed and refractory diffuse large B-cell and mantle cell lymphomas. Future Oncol. Jan. 15 2021; doi:10.2217/fon-2020-0291
16. Smith-Garvin J E, Koretzky G A, Jordan M S. T cell activation. Annual review of immunology. 2009; 27:591-619. doi:10.1146/annurev.immunol.021908.132706
17. Santomasso B D, Nastoupil L J, Adkins S, et al. Management of Immune-Related Adverse Events in Patients Treated With Chimeric Antigen Receptor T-Cell Therapy: ASCO Guideline. J Clin Oncol. Dec. 10 2021; 39(35):3978-3992. doi:10.1200/JCO.21.01992
18. Liu Y, Liu G, Wang J, et al. Chimeric STAR receptors using TCR machinery mediate robust responses against solid tumors. Science translational medicine. Mar. 24 2021; 13(586)doi:10.1126/scitranslmed.abb5191
19. Chung H, Jung H, Noh J Y. Emerging Approaches for Solid Tumor Treatment Using CAR-T Cell Therapy. Int J Mol Sci. Nov. 9 2021; 22(22)doi:10.3390/ijms222212126
20. Safarzadeh Kozani P, Safarzadeh Kozani P, Rahbarizadeh F. Novel antigens of CAR T cell therapy: New roads; old destination. Transl Oncol. July 2021; 14(7):101079. doi:10.1016/j.tranon.2021.101079
21. Minutolo N G, Hollander E E, Powell D J, Jr. The Emergence of Universal Immune Receptor T Cell Therapy for Cancer. Front Oncol. 2019; 9:176. doi:10.3389/fonc.2019.00176
22. Urbanska K, Lanitis E, Poussin M, et al. A universal strategy for adoptive immunotherapy of cancer through use of a novel T-cell antigen receptor. Cancer research. Apr. 1 2012; 72(7):1844-52. doi:10.1158/0008-5472.CAN-11-3890
23. Lee Y G, Marks I, Srinivasarao M, et al. Use of a Single CAR T Cell and Several Bispecific Adapters Facilitates Eradication of Multiple Antigenically Different Solid Tumors. Cancer research. Jan. 15 2019; 79(2):387-396. doi:10.1158/0008-5472.CAN-18-1834
24. Cao Y, Rodgers D T, Du J, et al. Design of Switchable Chimeric Antigen Receptor T Cells Targeting Breast Cancer. Angew Chem Int Ed Engl. Jun. 20 2016; 55(26):7520-4. doi:10.1002/anie.201601902
25. Minutolo N G, Sharma P, Poussin M, et al. Quantitative Control of Gene-Engineered T-Cell Activity through the Covalent Attachment of Targeting Ligands to a Universal Immune Receptor. J Am Chem Soc. Apr. 8 2020; 142(14):6554-6568. doi:10.1021/jacs.9b1 1622
26. Lake B, Serniuck N, Kapcan E, Wang A, Rullo A F. Covalent Immune Recruiters: Tools to Gain Chemical Control Over Immune Recognition. ACS Chem Biol. Apr. 17 2020; 15(4):1089-1095. doi:10.1021/acschembio.0c00112
27. Wang E, Wang L C, Tsai C Y, Bhoj V, Gershenson Z, Moon E, Newick K, Sun J, Lo A, Baradet T, Feldman M D, Barrett D, Pure E, Albelda S, Milone M C. Generation of Potent T-cell Immunotherapy for Cancer Using DAP12-Based, Multichain, Chimeric Immunoreceptors. Cancer Immunol Res. 2015 July; 3(7):815-26. doi: 10.1158/2326-6066.CIR-15-0054. Epub 2015 May 4.

1. A synthetic antigen receptor comprising:
an extracellular acceptor domain; and
an effector domain;
for use with a covalent immune recruiter (CIR) comprising a donor moiety, a covalent binding group, and a target binding domain, wherein the extracellular acceptor domain is capable of binding to the donor moiety of the CIR.

2. The synthetic antigen receptor of claim 1, wherein the extracellular acceptor domain comprises an antibody or antibody fragment, optionally an scFv.

3. The synthetic antigen receptor of claim 2, wherein the antibody fragment is an anti-DNP scFv and the donor moiety is DNP, optionally the scFv comprises a variable heavy chain fragment (VH) and a variable light chain fragment (VL) in a VH-VL orientation, optionally the scFv comprises an amino acid sequence of SEQ ID NO: 2 or a functional variant thereof.

4. The synthetic antigen receptor of claim 1, wherein the synthetic antigen receptor is a T cell antigen coupler (TAC)-based synthetic antigen receptor, a DNAX activation protein of 12 kDa (DAP12)-based synthetic antigen receptor, or a chimeric antigen receptor (CAR)-based synthetic antigen receptor.

5. The synthetic antigen receptor of claim 4, wherein the synthetic antigen receptor is:
a) a TAC-based synthetic antigen receptor and the effector domain comprises an extracellular CD3ε binding domain, a transmembrane domain, and a CD4 cytoplasmic domain, optionally wherein the effector domain comprises an amino acid sequence of SEQ ID NO: 6 or a functional variant thereof;
b) a DAP12-based synthetic antigen receptor and the effector domain comprises a KIR2DS2 transmembrane and hinge domain, an NKp44 transmembrane and hinge domain, a TREM-1 transmembrane and hinge domain, or a TREM-2 transmembrane and hinge domain, optionally wherein the effector domain comprises an amino acid sequence of SEQ ID NO: 10 or 12, or a functional variant thereof, and/or further comprising a DAP12 signaling protein, optionally comprising an amino acid sequence of SEQ ID NO: 8 or a functional variant thereof; or
c) a CAR-based synthetic antigen receptor and the effector domain comprises a transmembrane domain and an intracellular CD28 or CD137 signaling domain, optionally wherein the effector domain comprises an amino acid sequence of SEQ ID NO: 14 or 16, or a functional variant thereof.

6. (canceled)

7. The synthetic antigen receptor of claim 5, wherein the TAC-based synthetic antigen receptor comprises an amino acid sequence of SEQ ID NO: 18 or a functional variant thereof.

8. (canceled)

9. (canceled)

10. (canceled)

11. The synthetic antigen receptor of claim 5, wherein the DAP12-based synthetic antigen receptor comprises an amino acid sequence of SEQ ID NO: 22 or 24, or a functional variant thereof.

12. (canceled)

13. (canceled)

14. The synthetic antigen receptor of claim 5, wherein the CAR-based synthetic antigen receptor comprises an amino acid sequence of SEQ ID NO: 26 or 28, or a functional variant thereof.

15. An engineered eukaryotic cell comprising the synthetic antigen receptor of claim 1.

16. The cell of claim 15, wherein the cell is an immune cell selected from lymphocytes, monocytes, polymorphonuclear cells, erythrocytes and megakaryocytes.

17. (canceled)

18. The cell of claim 16, wherein the immune cell is selected from T cells, B cells, NK cells, macrophages, neutrophils, basophils, eosinophils and red blood cells.

19. The cell of claim 18, wherein the T cell is selected from alpha-beta T cells, gamma-delta T cells, Mucosal-associated invariant T cells and natural killer T cells.

20. The cell of claim 15, wherein the cell is further engineered to express one or more additional signaling components, optionally DAP12.

21. A method of generating a functionalized cell, the method comprising:
providing the cell of claim 15; and
contacting the cell with a CIR comprising a donor moiety that binds the acceptor domain, a covalent binding group, and a target binding domain under conditions to allow binding of the donor moiety and acceptor domain and covalent attachment of the covalent binding group to the synthetic antigen receptor;
thereby generating a functionalized cell.

22. A functionalized cell generated using the method of claim 21.

23. A nucleic acid encoding the synthetic antigen receptor of claim 1.

24. The nucleic acid of claim 23, wherein the extracellular acceptor domain comprises an scFv encoded by SEQ ID NO: 1 or a functional variant thereof.

25. The nucleic acid of claim 23, wherein the effector domain is encoded by SEQ ID NO: 5, 7, 9, 11, 13, or 15, or a functional variant thereof.

26. The nucleic acid of claim 23, comprising a sequence of SEQ ID NO: 17, 19, 21, 23, 25, or 27, or a functional variant thereof.

27. A method of treating a disease, disorder or condition that is treatable by immunotherapy, comprising administering to a subject in need thereof a therapeutically effective amount of
the engineered cell of claim 15, and a CIR comprising a donor moiety that binds the acceptor domain, a covalent binding group, and a target binding domain.

28. The method of claim 27, wherein the disease, disorder, or condition that is treatable by immunotherapy is a cancer, an autoimmune disease, allergy, or transplant rejection.

29. (canceled)

30. (canceled)

31. A method of generating an engineered cell comprising a synthetic antigen receptor for use with a covalent immune recruiter (CIR) comprising a donor moiety, a covalent binding group, and a target binding domain, the method comprising introducing into the cell the nucleic acid of claim 23, optionally wherein the nucleic acid is introduced into the cell by
a) viral transduction, optionally wherein the virus is selected from adenovirus, adeno associated virus, retrovirus, and lentivirus; or
b) transfection.

32. (canceled)

33. (canceled)

34. (canceled)

35. (canceled)

36. (canceled)

37. A method of treating a disease, disorder or condition that is treatable by immunotherapy, comprising administering to a subject in need thereof a therapeutically effective amount of the functionalized cell of claim 22.
